(19) United States
(12) Patent Application Publication
(10) Pub. No.: US 2023/0399408 A1
(43) Pub. Date: Dec. 14, 2023

(54) MULTISPECIFIC ANTIBODIES AND USES THEREOF

(71) Applicant: Ab Therapeutics, Inc., Hayward, CA (US)

(72) Inventors: Mohd Saif Zaman, San Francisco, CA (US); Yue Liu, Foster City, CA (US)

(21) Appl. No.: 18/036,200

(22) PCT Filed: Nov. 16, 2021
(86) PCT No.: PCT/US2021/059444

§ 371 Date: May 10, 2023

Related U.S. Application Data
(60) Provisional application No. 63/114,479, filed on Nov. 16, 2020.
(60) Provisional application No. 63/237,526, filed on Aug. 26, 2021.

(51) Int. Cl. C07K 16/28; A61P 35/00

(52) U.S. Cl. CPC C07K 16/2863; A61P 35/00; C07K 2317/24; C07K 2317/31; C07K 2317/622; C07K 2317/76; C07K 2317/33; C07K 2317/22; C07K 2317/92; A61K 2039/505

(57) ABSTRACT

This disclosure relates to multi specific antibodies (e.g., bi specific antibodies) or antigen binding fragments thereof. In one aspect, the multispecific antibodies or antigen-binding fragments thereof binds to cMET and/or EGFR, or a combination thereof. An antibody or antigen-binding fragment thereof that binds to cMET (tyrosine-protein kinase Met), comprising: a heavy-chain antibody variable domain (VHH) comprising complementarity determining regions (CDRs) 1, 2, and 3.

### CLAIM OF PRIORITY

This application claims the benefit of U.S. Provisional Application No. 63/114,479, filed on Nov. 16, 2020 and U.S. Provisional Application No. 63/237,526, filed on Aug. 26, 2021. The entire contents of the foregoing applications are incorporated herein by reference.

### TECHNICAL FIELD

This disclosure relates to multispecific antibodies or antigen-binding fragments thereof.

### BACKGROUND

A multispecific antibody is an artificial protein that can simultaneously bind to two or more different epitopes. This opens up a wide range of applications, including redirecting T cells to tumor cells, blocking two different signaling pathways simultaneously, dual targeting of different disease mediators, and delivering payloads to targeted sites. The approval of catumaxomab (anti-EpCAM and anti-CD3) and blinatumomab (anti-CD19 and anti-CD3) has become a major milestone in the development of multispecific antibodies.

As multispecific antibodies have various applications, there is a need to continue to develop various therapeutics based on multispecific antibodies.

### SUMMARY

This disclosure relates to antibodies or antigen-binding fragments, wherein the antibodies or antigen-binding fragments specifically bind to cMET and/or EGFR, or a combination thereof. In some embodiments, the disclosure relates to development of cMET/EGFR targeting bispecific antibodies. In some embodiments, the antibodies described herein can treat non-small cell lung cancer (NSCLC).

In one aspect, the disclosure is related to an antibody or antigen-binding fragment thereof that binds to cMET (tyrosine-protein kinase Met), comprising: a heavy-chain antibody variable domain (VHH) comprising complementarity determining regions (CDRs) 1, 2, and 3.

In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

In some embodiments, the VHH comprises CDRs 1, 2, 3 with the amino acid sequences set forth in SEQ ID NOs: 1, 2, and 3, respectively.

In some embodiments, the VHH comprises CDRs 1, 2, 3 with the amino acid sequences set forth in SEQ ID NOs: 4, 5, and 6, respectively.

In some embodiments, the VHH comprises CDRs 1, 2, 3 with the amino acid sequences set forth in SEQ ID NOs: 7, 8, and 9, respectively.

In some embodiments, the VHH comprises CDRs 1, 2, 3 with the amino acid sequences set forth in SEQ ID NOs: 10, 11, and 12, respectively.

In one aspect, the disclosure is related to an antibody or antigen-binding fragment thereof that binds to cMET comprising a heavy-chain antibody variable domain (VHH) comprising an amino acid sequence that is at least 80% identical to a selected VHH sequence. In some embodiments, the selected VHH sequence is selected from the group consisting of SEQ ID NOs: 19-22 and 77-84. In some embodiments, the VHH comprises the sequence of SEQ ID NO: 19, 77, 78, 79, 80, 81, or 84. In some embodiments, the VHH comprises the sequence of SEQ ID NO: 20. In some embodiments, the VHH comprises the sequence of SEQ ID NO: 21. In some embodiments, the VHH comprises the sequence of SEQ ID NO: 22, 82, or 83.

In some embodiments, the antibody or antigen-binding fragment specifically binds to cMET.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof as described herein.

In one aspect, the disclosure is related to a multi-specific antibody or antigen-binding fragment thereof, comprising a first antigen-binding site that specifically binds to EGFR, and a second antigen-binding site that specifically binds to cMET.

In some embodiments, the first antigen-binding site comprises a heavy chain variable region (VH) and a light chain variable region (VL). In some embodiments, the VH and the VL associate with each other and specifically bind to EGFR.

In some embodiments, the heavy chain variable region (VH) comprising complementarity determining regions (CDRs) 1, 2, and 3. In some embodiments, the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15. In some embodiments, the light chain variable region (VL) comprising CDRs 1, 2, and 3. In some embodiments, the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

In some embodiments, the heavy chain variable region (VH) comprises an amino acid sequence that is at least 90% identical to SEQ ID NO: 23, and a light chain variable region (VL) comprising an amino acid sequence that is at least 90% identical to SEQ ID NO: 24.

In some embodiments, the second antigen-binding site specifically binds to cMET, and the second antigen-binding site comprises a first heavy-chain antibody variable domain (VHH1) comprising complementarity determining regions (CDRs) 1, 2, and 3. In some embodiments, the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence. In some embodiments, the selected VHH1 CDRs 1, 2, and 3 amino acid sequences are one of the following:
(1) the selected VHH1 CDRs 1, 2, 3 amino acid sequences are set forth in SEQ ID NOs: 1, 2, and 3, respectively;
(2) the selected VHH1 CDRs 1, 2, 3 amino acid sequences are set forth in SEQ ID NOs: 4, 5, and 6, respectively;
(3) the selected VHH1 CDRs 1, 2, 3 amino acid sequences are set forth in SEQ ID NOs: 7, 8, and 9, respectively; and
(4) the selected VHH1 CDRs 1, 2, 3 amino acid sequences are set forth in SEQ ID NOs: 10, 11, and 12, respectively.

In some embodiments, the first heavy-chain antibody variable domain (VHH1) comprises an amino acid sequence that is at least 80% identical to a selected VHH sequence. In some embodiments, the selected VHH sequence is selected from the group consisting of SEQ ID NOs: 19-22 and 77-84. In some embodiments, the multi-specific antibody or antigen-binding fragment thereof as described herein further comprises a third antigen-binding site that specifically binds to cMET.

In some embodiments, the third antigen-binding site specifically binds to cMET, and the third antigen-binding site comprises a second heavy-chain antibody variable domain (VHH2) comprising complementarity determining regions (CDRs) 1, 2, and 3. In some embodiments, the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence. In some embodiments, the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

In some embodiments, the second heavy-chain antibody variable domain (VHH2) comprises an amino acid sequence that is at least 80% identical to a selected VHH2 sequence. In some embodiments, the selected VHH2 sequence is selected from the group consisting of SEQ ID NOs: 19-22 and 77-84.

In some embodiments, the VH and the VL are linked by a linker peptide sequence to form an scFv. In some embodiments, the linker peptide sequence comprises a sequence that is at least 80% identical to any one of SEQ ID NOs: 37-42.

In one aspect, the disclosure is related to a polypeptide complex, comprising (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to cMET. In some embodiments, the VH and the VL associate with each other and specifically bind to EGFR.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 43 or 44. In some embodiments, the second polypeptide comprises a sequence that is at least 80% identical to SEQ ID NO: 45 or 46. In some embodiments, the third polypeptide comprises a sequence that is at least 80% identical to SEQ ID NO: 47.

In some embodiments, the first polypeptide further comprises a second VHH that specifically binds to cMET. In some embodiments, the second VHH is linked to the N-terminus of the first VHH via a linker peptide sequence.

In some embodiments, the linker peptide sequence comprises a sequence that is at least 80% identical to any one of SEQ ID NOs: 37-42.

In some embodiments, the first polypeptide comprises a sequence that is at least 80% identical to SEQ ID NO: 48 or 49. In some embodiments, the second polypeptide comprises a sequence that is at least 80% identical to SEQ ID NO: 50 or 51. In some embodiments, the third polypeptide comprises a sequence that is at least 80% identical to SEQ ID NO: 52.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region. In some embodiments, the first VHH specifically binds to cMET. In some embodiments, the scFv comprises a heavy chain variable region (VH), a first linker peptide sequence, and a light chain variable region (VL). In some embodiments, the VH and the VL associate with each other and specifically bind to EGFR.

In some embodiments, the first linker peptide sequence comprises a sequence that is at least 80% identical to any one of SEQ ID NOs: 37-42.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 53 or 54. In some embodiments, the second polypeptide comprises a sequence that is at least 80% identical to SEQ ID NO: 55 or 56.

In some embodiments, the first polypeptide further comprises a second VHH that specifically binds to cMET. In some embodiments, the second VHH is linked to the N-terminus of the first VHH via a second linker peptide sequence.

In some embodiments, the second linker peptide sequence comprises a sequence that is at least 80% identical to any one of SEQ ID NOs: 37-42.

In some embodiments, the first polypeptide comprises a sequence that is at least 80% identical to SEQ ID NO: 57 or 58. In some embodiments, the second polypeptide comprises a sequence that is at least 80% identical to SEQ ID NO: 59 or 60.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80% identical to any one of SEQ ID NOs: 37-42.

In some embodiments, the first hinge region and/or the second hinge regions comprise a sequence that is at least 80% identical to any one of SEQ ID NOs: 25-29.

In some embodiments, sequences of the VHH1 and the VHH2 are identical. In some embodiments, the first Fc region and/or the second Fc region comprise a sequence that is at least 80% identical to SEQ ID NO: 32. In some embodiments, the first polypeptide comprises a sequence that is at least 80% identical to SEQ ID NO: 61 or 62. In some embodiments, the second polypeptide comprises a sequence that is at least 80% identical to SEQ ID NO: 63. In some embodiments, the third polypeptide comprises a sequence that is at least 80% identical to SEQ ID NO: 61 or 62. In some embodiments, the fourth polypeptide comprises a sequence that is at least 80% identical to SEQ ID NO: 63.

In some embodiments, sequences of the VHH1 and the VHH2 are different. In some embodiments, the first Fc region and/or the second Fc region comprise a sequence that is at least 80% identical to SEQ ID NO: 30 or 31. In some embodiments, the first polypeptide comprises a sequence that is at least 80% identical to SEQ ID NO: 64 or 65. In some embodiments, the second polypeptide comprises a sequence that is at least 80% identical to SEQ ID NO: 68. In some embodiments, the third polypeptide comprises a sequence that is at least 80% identical to SEQ ID NO: 66 or 67. In some embodiments, the fourth polypeptide comprises a sequence that is at least 80% identical to SEQ ID NO: 68. In some embodiments, the first polypeptide comprises a sequence that is at least 80% identical to SEQ ID NO: 85. In some embodiments, the second polypeptide comprises a sequence that is at least 80% identical to SEQ ID NO: 68. In some embodiments, the third polypeptide comprises a sequence that is at least 80% identical to SEQ ID NO: 86. In some embodiments, the fourth polypeptide comprises a sequence that is at least 80% identical to SEQ ID NO: 68.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80% identical to any one of SEQ ID NOs: 37-42.

In some embodiments, the first hinge region and/or the second hinge regions comprise a sequence that is at least 80% identical to any one of SEQ ID NOs: 25-29.

In some embodiments, sequences of the VHH1 and the VHH2 are identical. In some embodiments, the first Fc region and/or the second Fc region comprise a sequence that is at least 80% identical to SEQ ID NO: 32. In some embodiments, the first polypeptide comprises a sequence that is at least 80% identical to SEQ ID NO: 69 or 70. In some embodiments, the second polypeptide comprises a sequence that is at least 80% identical to SEQ ID NO: 71. In some embodiments, the third polypeptide comprises a sequence that is at least 80% identical to SEQ ID NO: 69 or 70. In some embodiments, the fourth polypeptide comprises a sequence that is at least 80% identical to SEQ ID NO: 71.

In some embodiments, sequences of the VHH1 and the VHH2 are different. In some embodiments, the first Fc region and/or the second Fc region comprise a sequence that is at least 80% identical to SEQ ID NO: 30 or 31. In some embodiments, the first polypeptide comprises a sequence that is at least 80% identical to SEQ ID NO: 72 or 73. In some embodiments, the second polypeptide comprises a sequence that is at least 80% identical to SEQ ID NO: 76. In some embodiments, the third polypeptide comprises a sequence that is at least 80% identical to SEQ ID NO: 74 or 75. In some embodiments, the fourth polypeptide comprises a sequence that is at least 80% identical to SEQ ID NO: 76.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to cMET. In some embodiments, the VH and the VL associate with each other and specifically bind to EGFR. In some embodiments, the first polypeptide further comprises a second VHH that specifically binds to cMET. In some embodiments, the second VHH is linked to the N-terminus of the first VHH via a linker peptide sequence.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the linker peptide sequence comprises a sequence that is at least 80% identical to any one of SEQ ID NOs: 37-42.

In some embodiments, the first polypeptide comprises a sequence that is at least 80% identical to SEQ ID NO: 48 or 49. In some embodiments, the second polypeptide comprises a sequence that is at least 80% identical to SEQ ID NO: 50 or 51. In some embodiments, the third polypeptide comprises a sequence that is at least 80% identical to SEQ ID NO: 52.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region. In some embodiments, the first VHH specifically binds to cMET. In some embodiments, the scFv comprises a heavy chain variable region (VH), a first linker peptide sequence, and a light chain variable region (VL). In some embodiments, the VH and the VL associate with each other and specifically bind to EGFR. In some embodiments, the first polypeptide further comprises a second VHH that specifically binds to cMET. In some embodiments, the second VHH is linked to the N-terminus of the first VHH via a second linker peptide sequence.

In some embodiments, the first linker peptide sequence and/or the second linker peptide sequence comprise a sequence that is at least 80% identical to any one of SEQ ID NOs: 37-42.

In some embodiments, the first hinge region and/or the second hinge region comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 57 or 58. In some embodiments, the second polypeptide comprises a sequence that is at least 80% identical to SEQ ID NO: 59 or 60.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to cMET. In some embodiments, the VH1 and the VL1 associate with each other and specifically bind to EGFR. In some embodiments, the VH2 and the VL2 associate with each other and specifically bind to EGFR. In some embodiments, sequences of the VHH1 and the VHH2 are different.

In some embodiments, the first linker peptide sequence and/or the second linker peptide sequence comprise a sequence that is at least 80% identical to any one of SEQ ID NOs: 37-42.

In some embodiments, the first hinge region and/or the second hinge regions comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 64 or 65. In some embodiments, the second polypeptide comprises a sequence that is at least 80% identical to SEQ ID NO: 68. In some embodiments, the third polypeptide comprises a sequence that is at least 80% identical to SEQ ID NO: 66 or 67. In some embodiments, the fourth polypeptide comprises a sequence that is at least 80% identical to SEQ ID NO: 68.

In some embodiments, the first polypeptide comprises a sequence that is at least 80% identical to SEQ ID NO: 85. In some embodiments, the second polypeptide comprises a sequence that is at least 80% identical to SEQ ID NO: 68. In some embodiments, the third polypeptide comprises a sequence that is at least 80% identical to SEQ ID NO: 86. In some embodiments, the fourth polypeptide comprises a sequence that is at least 80% identical to SEQ ID NO: 68.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a polypeptide complex, comprising (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to cMET. In some embodiments, the VH1 and the VL1 associate with each other and specifically bind to EGFR. In some embodiments, the VH2 and the VL2 associate with each other and specifically bind to EGFR. In some embodiments, sequences of the VHH1 and the VHH2 are different.

In some embodiments, the first linker peptide sequence and/or the second linker peptide sequence comprises a sequence that is at least 80% identical to any one of SEQ ID NOs: 37-42.

In some embodiments, the first hinge region and/or the second hinge regions comprise a sequence that is at least 80% identical to any one of SEQ ID NOs: 25-29.

In some embodiments, the first Fc region and/or the second Fc region comprise a sequence that is at least 80% identical to SEQ ID NO: 30 or 31.

In some embodiments, the first polypeptide comprises a sequence that is at least 80% identical to SEQ ID NO: 72 or 73. In some embodiments, the second polypeptide comprises a sequence that is at least 80% identical to SEQ ID NO: 76. In some embodiments, the third polypeptide comprises a sequence that is at least 80% identical to SEQ ID NO: 74 or 75. In some embodiments, the fourth polypeptide comprises a sequence that is at least 80% identical to SEQ ID NO: 76.

In some embodiments, the first Fc region and/or the second Fc region comprises an aspartic acid (Asp) at position 239 according to EU numbering, and/or a glutamic acid (Glu) at position 332 according to EU numbering.

In one aspect, the disclosure is related to a nucleic acid comprising a polynucleotide encoding the antibody or antigen-binding fragment thereof as described herein, the multi-specific antibody or antigen-binding fragment thereof as described herein, or the polypeptide complex as described herein. In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein. In some embodiments, the cell is a CHO cell.

In one aspect, the disclosure is related to a cell comprising one or more of the nucleic acids as described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody-drug conjugate comprising the antibody or antigen-binding fragment thereof as described herein, the multi-specific antibody or antigen-binding fragment thereof as described herein, or the polypeptide complex as described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof as described herein, the multi-specific antibody or antigen-binding fragment thereof as described herein, the polypeptide complex as described herein, or the antibody-drug conjugate as described herein, to the subject.

In some embodiments, the subject has a cancer expressing cMET. In some embodiments, the subject has a cancer expressing EGFR.

In some embodiments, the cancer is lung cancer. In some embodiments, the cancer is non-small cell lung cancer (NSCLC).

In one aspect, the disclosure is related to a method of decreasing the rate of tumor growth, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a pharmaceutical composition comprising the antibody or antigen-binding fragment thereof as described herein, the multi-specific antibody or antigen-binding fragment thereof as described herein, the polypeptide complex as described herein, and a pharmaceutically acceptable carrier.

In one aspect, the disclosure is related to a pharmaceutical composition comprising the antibody-drug conjugate as described herein, and a pharmaceutically acceptable carrier.

In one aspect, the disclosure provides a multi-specific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a bispecific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a multi-specific antibody or antigen-binding fragment thereof that specifically binds to cMET. In one aspect, the disclosure provides a multi-specific antibody or antigen-binding fragment thereof that specifically binds to EGFR.

In one aspect, the disclosure provides a multispecific antibody targeting both cMET and EGFR. In some embodiments, the multispecific antibodies described herein has a comparable or even higher binding capabilities to cMET and/or EGFR as compared to JNJ372 or JNJ372 analog. In some embodiments, the multispecific antibodies described herein is more effective than JNJ372 or JNJ372 analog in blocking the interaction of cMET with a cMET ligand (e.g., HGF). In some embodiments, the multispecific antibodies described herein is more effective than JNJ372 analog in blocking interaction of EGFR with an EGFR ligand (e.g., EGF). In some embodiments, the multispecific antibodies described herein is comparable or even more effective in inhibiting phosphorylation of cMET and/or EGFR, as compared to JNJ372 or JNJ372 analog. In some embodiments, the multispecific antibodies described herein is more effective in inhibiting phosphorylation of the downstream signaling pathways (e.g., ERK and/or Akt pathways) involved in cancer cell survival and proliferation as compared to JNJ372 or JNJ372 analog.

The present disclosure provides multispecific antibodies or antigen-binding fragments thereof that binds to EGFR and cMET. A multi-specific antibody (e.g., bispecific antibody or a trispecific antibody) or antigen-binding fragment thereof is an artificial protein that can simultaneously bind to two or more different types of epitopes. The epitopes can be in the same antigen or in different antigens. In some embodiments, a multi-specific antibody or antigen-binding fragment thereof can have two, three, four, five, six, or more antigen binding sites. In some embodiments, the antigen binding site has one heavy chain variable region and one light chain variable region. In some embodiments, the antigen binding site has one VHH.

The present disclosure provides multispecific antibodies or antigen-binding fragments thereof that binds to EGFR and cMET. The present disclosure further provides anti-cMET VHHs, and anti-EGFR antibodies having a VH and a VL, or antigen binding fragments thereof. These VHH, VH, VL can be used to make various multispecific antibodies or antigen-binding fragments as described herein.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to EGFR may be referred to as a EGFR-specific antibody or an anti-EGFR antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows luminescent signals of a competitive ELISA assay. Binding between cMET and Hu-HGF in the presence of 16 unique clones were determined. RFU is average relative fluorescence unit.

FIG. 2 shows cross reactivity of 16 unique clones to human cMET (anti-Hu) and monkey cMET (anti-Cyno).

FIG. 3 shows the VHH expression levels of 1D5, 1F3, 1H1 and 2C7 clones determined by Bio-Layer Interferometry (BLI).

FIG. 4 shows ELISA results of VHH proteins binding to cMET. Media and PBS are negative controls.

FIG. 5 shows luminescent signals of a competitive ELISA assay. Binding between cMET and Hu-HGF in the presence of cMET VHH clones 1D5, 1H1, and 2C7 were determined. 11F11 is an isotype control.

FIG. 6 shows a schematic structure of 1H1-cMET/EGFR-Fc(WT)-V3.

FIG. 7 shows a cMET-binding ELISA result of 1H1-cMET/EGFR-Fc(WT)-V3. JNJ372 analog (JNJ BsAb) is a positive control. 11F11 is an isotype negative control.

FIG. 8 shows a EGFR-binding ELISA result of 1H1-cMET/EGFR-Fc(WT)-V3. JNJ372 analog (JNJ BsAb) is a positive control. 11F11 is an isotype negative control.

FIG. 9 shows titration ELISA results for binding to cMET of 1H1-cMET/EGFR-Fc(WT)-V1 (1H1 v1), 1H1-cMET/EGFR-Fc(WT)-V3 (1H1 v3), and JNJ372 analog.

FIG. 10 shows titration ELISA results for binding to EGFR of 1H1-cMET/EGFR-Fc(WT)-V3 (1H1 v3), and JNJ372 analog.

FIG. 11 shows luminescent signals of a competitive ELISA assay. Binding between cMET and Hu-HGF in the presence of EGFR of 1H1-cMET/EGFR-Fc(WT)-V3 and JNJ372 analog were determined. 11F11 is an isotype control. Hu-HGF-B is a biotinylated human HGF control without adding any antibody.

FIG. 12A shows EGF/EGFR blocking effects of EGFR of 1H1-cMET/EGFR-Fc(WT)-V3 and JNJ372 analog. EGF-B is biotinylated EGF.

FIG. 12B shows an immuno-fluorescent image of NCIH1975 cells treated with PBS only.

FIG. 12C shows an immuno-fluorescent image of NCIH1975 cells incubated with 11F11, and then treated with biotinylated EGF.

FIG. 12D shows an immuno-fluorescent image of NCIH1975 cells treated with biotinylated EGF only.

FIG. 12E shows an immuno-fluorescent image of NCIH1975 cells incubated with JNJ372 analog, and then treated with biotinylated EGF.

FIG. 12F shows an immuno-fluorescent image of NCIH1975 cells incubated with 1H1-cMET/EGFR-Fc(WT)-V3, and then treated with biotinylated EGF.

FIG. 13A shows percentage of NCIH1975 cells with phosphorylated cMET. The cells were treated with JNJ372 analog or 1H1-cMET/EGFR-Fc(WT)-V3, then treated with HGF. HGF is a positive control without any antibody treatment. NC is an isotype control.

FIG. 13B shows percentage of NCIH1975 cells with phosphorylated EGFR. The cells were treated with JNJ372 analog or 1H1-cMET/EGFR-Fc(WT)-V3, then treated with EGF. “EGF+No Ab” is a positive control without any antibody treatment. NC is an isotype control.

FIG. 14A shows the ratio of phosphorylated ERK (pERK) in NCI-H1975 cells grown in the presence of Hu-HGF. The cells were treated with JNJ372 analog or 1H1-cMET/EGFR-Fc(WT)-V3.

FIG. 14B shows the ratio of phosphorylated Akt (pAkt) in NCI-H1975 cells grown in the presence of Hu-HGF. The cells were treated with JNJ372 analog or 1H1-cMET/EGFR-Fc(WT)-V3.

FIG. 15A shows the ADCC effects of JNJ372 analog and 1H1-cMET/EGFR-Fc(WT)-V3.

FIG. 15B shows the CDC effects of JNJ372 analog and 1H1-cMET/EGFR-Fc(WT)-V3.

FIG. 15C shows the cancer cell killing effects of JNJ372 analog and 1H1-cMET/EGFR-Fc(WT)-V3 (1H1 v3).

FIG. 16A shows a schematic structure of an exemplary cMET/EGFR bispecific antibody with the BiSpecific-V1 format.

FIG. 16B shows a schematic structure of an exemplary cMET/EGFR trispecific antibody with the TriSpecific-V1 format.

FIG. 17A shows a schematic structure of an exemplary cMET/EGFR bispecific antibody with the BiSpecific-V2 format.

FIG. 17B shows a schematic structure of an exemplary cMET/EGFR trispecific antibody with the TriSpecific-V2 format.

FIG. 18A shows a schematic structure of an exemplary cMET/EGFR bispecific antibody with the BiSpecific-V3 format.

FIG. 18B shows a schematic structure of an exemplary cMET/EGFR trispecific antibody with the TriSpecific-V3 format.

FIG. 19A shows a schematic structure of an exemplary cMET/EGFR bispecific antibody with the BiSpecific-V4 format.

FIG. 19B shows a schematic structure of an exemplary cMET/EGFR trispecific antibody with the TriSpecific-V4 format.

FIG. 20A shows percentage of cells stained by a pHrodo-labeled secondary antibody after incubating with JNJ372 analog, 1H1-cMET/EGFR-Fc(WT)-V3, or 1H1-cMET/EGFR-Fc(WT)-V3 Tri.

FIG. 20B shows images of cells stained by a pHrodo-labeled secondary antibody after after incubating with JNJ372 analog, 1H1-cMET/EGFR-Fc(WT)-V3, or 1H1-cMET/EGFR-Fc(WT)-V3 Tri.

FIG. 21A shows the cMET VHH-Fc epitope binning assay result for humanized VHH of cMET-1H1 (SEQ ID NO: 19).

FIG. 21B shows the cMET VHH-Fc epitope binning assay result for VHH of cMET-1A12 (SEQ ID NO: 20).

FIG. 22A shows the antibody binding curves to H1975 cells and the calculated EC50 values. Anti-His IgG was used as a negative control.

FIG. 22B shows the antibody binding curves to H1975.HGF cells and the calculated EC50 values. Anti-His IgG was used as a negative control.

FIG. 22C shows the antibody binding curves to EGFR mutant C797S-BaF3 cells and the calculated EC50 values. Anti-His IgG was used as a negative control.

FIG. 23A shows the antibody blocking curves to EGF/EGFR interaction using H1975 cells and the calculated EC50 values. Anti-His IgG was used as a negative control.

FIG. 23B shows the antibody blocking curves to EGF/EGFR interaction using H1975.HGF cells and the calculated EC50 values. Anti-His IgG was used as a negative control.

FIG. 23C shows the antibody blocking curves to EGF/EGFR interaction using EGFR mutant C797S-BaF3 cells and the calculated EC50 values. Anti-His IgG was used as a negative control.

FIG. 24A shows the antibody blocking curves to cMET/EGF interaction using H1975 cells and the calculated EC50 values. Anti-His IgG was used as a negative control.

FIG. 24B shows the antibody blocking curves to cMET/EGF interaction using H1975.HGF cells and the calculated EC50 values. Anti-His IgG was used as a negative control.

FIG. 25A shows a set of Western blot results using H1975 cells (HGF−) that were stimulated with HGF. The cells were also treated with 1H1cMET/1A12cMET/EGFR(WT) (“Trispecific”), 1H1-cMET/EGFR-Fc(WT)-V3 (“Bispecific”), JNJ372 analog (“JNJ”) or a negative control antibody (“Neg”). The concentrations of the antibodies were 0.16 μg/ml or 4 g/ml. The arrow highlights the inefficiency of JNJ373 analog in mediating EGFR degradation compared to that of the Trispecific.

FIG. 25B shows a set of Western blot results using H1975.HGF cells (HGF+). The cells were also treated with 1H1cMET/1A12cMET/EGFR(WT) (“TsAb”), 1H1-cMET/EGFR-Fc(WT)-V3 (“BsAb”), JNJ372 analog (“JNJ”) or a negative control antibody (“Neg”). The concentrations of the antibodies were 0.16 μg/ml or 4 μg/ml.

FIG. 25C shows a set of Western blot results using cell lines with untreatable EGFR point mutation C797S. The cells were also treated with 1H1cMET/1A12cMET/EGFR(WT) (“Trispecific Ab”), 1H1-cMET/EGFR-Fc(WT)-V3 (“Bi-specific Ab”), JNJ372 analog (“JNJ”), anti-His Tag antibody (“Anti-His”), or a negative control antibody (“Neg”). The concentrations of the antibodies were 0.16 μg/ml or 4 μg/ml.

FIG. 26A shows the cellular proliferation curves of H1975 cells when treated with antibodies. Anti-His IgG was used as a negative control.

FIG. 26B shows the cellular proliferation curves of H1975.HGF cells when treated with antibodies. Anti-His IgG was used as a negative control.

FIG. 26C shows the cellular proliferation curves of C797S-BaF3 cells when treated with antibodies. Anti-His IgG was used as a negative control.

FIG. 27A shows the antibody internalization curves in H1975 cells and the calculated IC50 values. Anti-His IgG was used as a negative control.

FIG. 27B shows the antibody internalization curves in C797S-BaF3 cells and the calculated IC50 values. Anti-His IgG was used as a negative control.

FIG. 27C shows the antibody internalization curves in H1975.HGF cells and the calculated IC50 values. Anti-His IgG was used as a negative control.

FIG. 27D shows the antibody internalization curves in D770-BaF3 cells and the calculated IC50 values. Anti-His IgG was used as a negative control.

FIG. 28A shows the ADCC assay results using H1975.GFP.Luc cells and PMBCs at a target:effector ratio of 1:10. IC50 values were also calculated for each tested antibody. Anti-His IgG was used as a negative control.

FIG. 28B shows the ADCC assay results using H1975.GFP.Luc.HGF cells and PMBCs at a target:effector ratio of 1:10. IC50 values were also calculated for each tested antibody. Anti-His IgG was used as a negative control.

FIG. 29A shows the design of the efficacy study. Tumor cell inoculation was performed on Day −20. Antibody treatment (Tx) by Intraperitoneal injection (IP) was performed on Day 0, Day 4, Day 8, Day 12, Day 15, and Day 19. Tumor volume (TV) was monitored twice a week after the first treatment. The experiment was terminated on Day 26. The “arm” in the 4-arm means the treatment in the study. “4-arm” means 4 treatments including the control.

FIG. 29B shows the group designation, dose levels, and dosing schedule.

FIG. 29C shows the absolute median tumor volume of Balb/c NU/Nu mice that were inoculated with H1975 tumor cells and then treated with JNJ372 analog or 1H1cMET/1A12cMET/EGFR(WT). DPBS was used a negative control.

FIG. 30A shows the design of the efficacy study. Tumor cell inoculation was performed on Day −20. Antibody treatment (Tx) by Intraperitoneal injection (IP) was performed on Day 0, Day 3, Day 7, Day 10, Day 14, Day 17, Day 21, and Day 24. Tumor volume (TV) was monitored twice a week after the first treatment. The experiment was terminated on Day 28. The “arm” in the 4-arm means the treatment in the study. “4-arm” means 4 treatments including the control.

FIG. 30B shows the group designation, dose levels, and dosing schedule.

FIG. 30C shows the mean tumor volume of Balb/c NU/Nu mice that were inoculated with H1975-Luc-GFP-HGF tumor cells and then treated with JNJ372 analog or 1H1cMET/1A12cMET/EGFR(WT). DPBS was used a negative control.

FIG. 31A shows the design of the efficacy study. Tumor cell inoculation was performed on Day −20. Antibody treatment (Tx) by Intraperitoneal injection (IP) was performed on Day 0, Day 3, Day 7, Day 10, Day 14, and Day 17. Tumor volume (TV) was monitored twice a week after the first treatment. The experiment was terminated on Day 22. The “arm” in the 4-arm means the treatment in the study. “4-arm” means 4 treatments including the control.

FIG. 31B shows the group designation, dose levels, and dosing schedule.

FIG. 31C shows the mean tumor volume of Balb/c NU/Nu mice that were inoculated with C797S-BaF3 cells and then treated with JNJ372 analog or 1H1cMET/1A12cMET/EGFR(WT). DPBS was used a negative control.

FIG. 32 lists CDR sequences of the VHHs from the cMET antibodies described in the disclosure.

FIG. 33 lists CDR sequences of the heavy chain variable region (VH) of the EGFR antibodies described in the disclosure.

FIG. 34 lists CDR sequences of the light chain variable region (VL) of the EGFR antibodies described in the disclosure.

FIG. 35 lists amino acid sequences of VHHs that specifically bind to cMET as described in the disclosure.

FIG. 36 lists amino acid sequences of VH and VL that specifically bind to EGFR as described in the disclosure.

FIG. 37 lists sequences discussed in the disclosure.

### DETAILED DESCRIPTION

Lung cancer is the leading cause of cancer-related mortality in the US for both genders. The incidence and mortality rates are highest in the developed countries. According to the World Health Organization (WHO), lung cancer death rates worldwide will continue to rise, primarily because of an increase in global tobacco use, particularly in Asia. NSCLC (Non-small Cell Lung Cancer) accounts for about 85% of all lung cancers. The 5-year overall survival rate for non-small cell lung cancer (NSCLC) remains poor, from 68% in patients with stage IB disease to 0% to 10% in patients with stage IVA-IVB disease. Thus, the development of an effective therapeutic agent against NSCLC is essential.

Therapeutic modalities against NSCLC can be broadly classified under two categories, immune checkpoint blockers (ICBs) and molecularly targeted therapies. Despite their remarkable clinical improvements, ICBs can lead to the development of primary and secondary resistance. Molecularly targeted therapies target oncogenic drivers of cancers. EGFR mutations are the most frequent mutations among targetable oncogenic drivers. On the other hand, anomalous c-Met signaling has been detected in many human cancers including NSCLC. Moreover, c-Met is a critical player in developing resistance to targeted therapies, including therapies directed at EGFR. Thus, targeting both EGFR and c-Met simultaneously could be an effective strategy.

In the past two decades the treatment of NSCLC and lung cancer in general has progressed from cytotoxic therapies to immunotherapy treatments. Early stage disease can be effectively treated with cytotoxic and surgical methods. However, a high percentage of tumors can recur, resulting in a low survival rate.

The development of more effective systemic therapies with the objective of improving the long-term survival rate has mainly entailed the use of immunotherapy and molecularly targeted therapies. A major advancement in the management of advanced stage NSCLC happened in 2015 with the approval of nivolumab (anti-PD-L1, an immune checkpoint blocker (ICB)) by the US FDA for patients whose disease progressed during or after platinum-based therapy. Notwithstanding the remarkable clinical improvements, most patients eventually fail to respond to ICB therapy due to the development of primary or secondary resistance. This might be because of several reasons including defects in cytokine signaling, MHC complex presentation, or increased levels of enzymes which catabolize tryptophan which is required for optimal T cell function.

Molecularly targeted therapies in NSCLC patients were started in the late 1990s with the use of gefitinib, an oral EGFR (Epidermal growth factor receptor) tyrosine kinase inhibitor (TKI). The physiological function of the epidermal growth factor receptor (EGFR) is to regulate epithelial tissue development and homeostasis. In lung and breast cancer and in glioblastoma, the EGFR is a driver of tumorigenesis. EGFR mutations are the most frequent mutations among targetable oncogenic drivers accounting for approximately 25% of NSCLC (Kris, M. G. et al., “Using multiplexed assays of oncogenic drivers in lung cancers to select targeted drugs.” Jama 311.19 (2014): 1998-2006). Activating mutations in EGFR, such as exon 19 del and L858R on exon 21, sensitize the majority of NSCLC tumor cells to the first-generation EGFR-TKIs gefitinib and erlotinib and the second-generation EGFR-TKIs afatinib and dacomitinib. Among these 50% patients with these tumors, develop acquired resistance due to the EGFR T790M mutation. This resistance led to the development of the third-generation EGFR-TKIs drugs osimertinib (TAGRISSO) and rociletinib. With the widespread use of osimertinib, the EGFR C797S resistance mutation appeared as well. In addition to secondary EGFR mutations, bypass mechanisms such as MET or ERBB2 amplification, Hippo pathway inhibition, and insulin-like growth factor 1 receptor (IGF1R) activation also contribute to resistance to EGFR-TKIs.

Aberrant cMET signaling has been implicated in the development/progression of many human cancers. This results from the overexpression of cMET, activating mutations in cMET, transactivation, autocrine or paracrine signaling, or cMET gene amplification. A significant relationship between EGFR and cMET signaling was recognized through the studies on cancer therapy outcomes. cMET is a critical player in developing resistance to targeted therapies, including therapies directed at EGFR. Similarly, EGFR and downstream gene mutations such as KRAS, histologic transformation, and the activation of alternative pathways, which includes the cMET signaling pathway, have been identified as mechanisms of resistance to EGFR-targeted therapies. Consequently, blocking one receptor tends to upregulate the other, leading to resistance to single-agent treatment. Amplification of cMET and/or high levels of HGF ligand expression have been observed in NSCLC patients with intrinsic or acquired resistance to tyrosine kinase inhibitors of EGFR, including erlotinib and gefitinib. Conversely, MET-amplified lung cancer cells exposed to cMET-inhibiting agents for a prolonged period develop resistance via the EGFR pathway.

Because of the signaling crosstalk between EGFR and cMet, inhibition of both receptors in combination may lead to improved outcomes for patients with MET- and EGFR-driven cancers. Additionally, concurrent inhibition may overcome or delay therapeutic resistance compared to the blockade of just one pathway. The dual inhibition of MET and EGFR has been explored using a combination of separate MET and EGFR inhibitors (bivalent MET antibody, emibetuzumab (LY2875358 Eli Lilly), as monotherapy and in combination with erlotinib in advanced cancer. But the developments of Bispecific antibodies (BsAbs) against both EGFR and cMET has been a major development in the treatment of NSCLC. Two major BsAbs have been tested (LY3164530 from Eli Lilly and JNJ-61186372 (Amivantamab, also known as JNJ372) from Janssen labs and Genmab). Details of LY3164530 and JNJ-61186372 can be found, e.g., in PCT/US2013/071288; Moores, S. L. et al., “A novel bispecific antibody targeting EGFR and cMet is effective against EGFR inhibitor-resistant lung tumors.” Cancer research 76.13 (2016): 3942-3953; and Patnaik, A. et al., “A phase I study of LY3164530, a bispecific antibody targeting MET and EGFR, in patients with advanced or metastatic cancer.” Cancer Chemotherapy and Pharmacology 82.3 (2018): 407-418; each of which is incorporated herein by reference.

The Phase I clinical trial (ClinicalTrials.gov Identifier: NCT02221882) of LY3164530 drug was started in August 2014 and the study was completed on Mar. 7, 2017. The main purpose of this study was to evaluate the safety of LY3164530 in participants with cancer that is advanced and/or has spread to other part(s) of the body. The drug LY3164530 did not make it to Phase II due to the following reasons a) lack of predictive markers for the study, b)) increased toxicity, c) PK and PD issues and d) dose adjustments due to adverse events. JNJ-61186372 was created under a collaboration between Genmab and Janssen Biotech, Inc. using Genmab's DuoBody® technology (Moores, S. L. et al., “A novel bispecific antibody targeting EGFR and cMet is effective against EGFR inhibitor-resistant lung tumors.” Cancer research 76.13 (2016): 3942-3953). JNJ-61186372 is being investigated in Phase II clinical studies to treat non-small cell lung cancer (NSCLC) ClinicalTrials.gov Identifier: NCT02609776. This study was started on May 24, 2016 and is an ongoing trial schedule to finish in July 2022. Additionally, this drug has been granted the U.S. FDA Breakthrough Therapy Designation for the Treatment of Non-Small Cell Lung Cancer.

The development of tumor-targeting small molecule kinase inhibitors, Osimertenib, and BsAbs like JNJ61186372 has led to improved progression-free survival for many NSCLC patients. However, there has been documentation of resistance to JNJ61186372 mono and combination therapies (in combination with Osimertenib, TAGRISSO) in in vivo mouse models. In a study done to identify signaling based mechanism of resistance (Goodman, A. M. et al., “Tumor mutational burden as an independent predictor of response to immunotherapy in diverse cancers.” Molecular Cancer Therapeutics 16.11 (2017): 2598-2608) subcutaneous cell line xenografts were treated with either Osimertinib, JNJ-61186372, or the combination, for either a short time to determine target inhibition or an extended period of time to establish resistant tumors, followed by mass spectrometry-based phosphoproteomic analysis of these resected tumors to quantify bypass signaling or target re-activation. In the short time study, Osimertenib was found to be more effective than JNJ-61186372 as it had the strongest effect on tumor phosphotyrosine profiles. All tumors treated with Osimertenib were more highly correlated and grouped together irrespective of the combination treatment. In contrast, tumors given JNJ-61186372 monotherapy demonstrated greater inter tumor heterogeneity and were poorly correlated with either Osimertenib monotherapy or the combination therapy. Additionally, tumors treated with Osimertenib monotherapy demonstrated a pronounced decrease in phosphorylation of EGFR, Shc1 and Gab1, indicating a strong on-target inhibition of EGFR as compared to a modest decrease in JNJ-61186372 monotherapy.

In this disclosure, a multispecific antibody (e.g., 1H1-cMET/EGFR-Fc(WT)-V3) was created targeting both cMET and EGFR. In some embodiments, the structural format of the antibody is such that it has two VHH domains which bind to cMET, whereas the main IgG structure binds to EGFR. The experiments showed that the multispecific antibody was more effective than the reference control bispecific antibody (e.g., JNJ372 analog from Janssen labs) in terms of its binding capabilities to cMET and/or EGFR receptors. Furthermore, the multispecific antibody described herein can also block the interaction between these receptors and their respective ligands. Functional assays revealed that the multispecific antibody is favorably comparable to JNJ372 analog and in most cases better in successfully inhibiting the phosphorylation of not only the cMET and EGFR receptors, but also the downstream signaling pathways by inhibiting the phosphorylation of ERK and Akt proteins, which are involved in cancer cell proliferation, survival and obstructing the apoptotic pathways. The multispecific antibody was also found to be more effective in complement dependent cytotoxicity (CDC) as compared to JNJ372 analog. Thus, these data support that the multispecific antibody described herein can be developed as a drug candidate for patients with lung cancer and other malignancies that are related to aberrant EGFR and cMET signaling.

Also in this disclosure, a trispecific antibody (e.g., 1H1cMET/1A12cMET/EGFR(WT)) was created which targets both cMET and EGFR. In some embodiments, the antibody has two separate VHH domains which bind to two different epitopes of cMET whereas the main IgG structure binds to EGFR. The experiments showed that the trispecific antibody was more effective than the benchmark control bispecific antibody (e.g., JNJ372 analog from Janssen labs) in terms of its binding capabilities to cMET and EGFR receptors. Furthermore, the trispecific antibody can also block the interaction between these receptors and their respective ligands. Functional assays revealed that the trispecific antibody is favorably comparable to JNJ372 analog in successfully inhibiting the phosphorylation of not only the cMET and EGFR receptors, but also the downstream signaling pathways by inhibiting the phosphorylation of MET and degrading EGFR proteins, which are involved in cancer cell proliferation, survival and obstructing the apoptotic pathways. Additionally, the trispecific antibody showed enhanced internalization of the receptors in comparison to the benchmark. Furthermore, the trispecific antibody was also found to be more effective in antibody-dependent cellular cytotoxicity as compared to JNJ372 analog. Moreover, in vivo results have shown the trispecific antibody to be more effective in tumor reduction than the JNJ372 analog. Thus, these data support that the trispecific antibody described herein can be developed as a drug candidate for patients with lung cancer and other malignancies that are related to aberrant EGFR and cMET signaling.

EGFR and cMET

Epidermal growth factor receptor (EGFR, ErbBI or HER1) is a Type 1 transmembrane glycoprotein of 170 kDa that is encoded by the c-erbB1 proto-oncogene. The epidermal growth factor receptor is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations affecting EGFR expression or activity could result in cancer. EGFR signaling is initiated by ligand binding followed by induction of conformational change, homodimerization or heterodimerization of the receptor with other ErbB family members, and trans-autophosphorylation of the receptor, which initiates signal transduction cascades that ultimately affect a wide variety of cellular functions, including cell proliferation and survival, increases in expression or kinase activity of EGFR have been linked with a range of human cancers, making EGFR an attractive target for therapeutic intervention. Increases in both the EGFR gene copy number and protein expression have been associated with favorable responses to the EGFR tyrosine kinase inhibitor, IRESSA™ (gefitinib), in non-small cell lung cancer.

c-Met, also called tyrosine-protein kinase Met or hepatocyte growth factor receptor (HGFR), is a protein that in humans is encoded by the MET gene. The protein possesses tyrosine kinase activity. The primary single chain precursor protein is post-translationally cleaved to produce the alpha and beta subunits, which are disulfide linked to form the mature receptor. Activation of c-Met by its ligand hepatocyte growth factor (HGF) stimulates a plethora of cell processes including growth, motility, invasion, metastasis, epithelial-rnesenchynial transition, angiogenesis/wound healing, and tissue regeneration. The exact stoichiometry of HGF:c-Met binding is unclear, but it is generally believed that two HGF molecules bind to two c-Met molecules leading to receptor dimerization and autophosphorylation at tyrosines 1230, 1234, and 1235. Ligand-independent c-Met autophospliorylation can also occur due to gene amplification, mutation or receptor over-expression.

c-Met is frequently amplified, mutated or over-expressed in many types of cancer including gastric, lung, colon, breast, bladder, head and neck, ovarian, prostate, thyroid, pancreatic, and CNS cancers. Missense mutations typically localized to the kinase domain are commonly found in hereditary papillary renal cell carcinomas (PRCC) and in 13% of sporadic PRCCs (Schmidt et al, Oncogene 18: 2343-2350, 1999), c-Met mutations localized to the semaphorin or juxtamembrane domains of c-Met are frequently found in gastric, head and neck, liver, ovarian, NSCLC and thyroid cancers. c-Met amplification has been detected in brain, colorectal, gastric, and lung cancers, often correlating with disease progression. Up to 4% and 20% of non-small cell lung cancer (NSCLC) and gastric cancers, respectively, exhibit c-Met amplification. c-Met overexpression is also frequently observed in lung cancer. Moreover, in clinical samples, nearly half of lung adenocarcinomas exhibited high levels of c-Met and HGF, both of which correlated with enhanced tumor growth rate, metastasis and poor prognosis.

Nearly 60% of all tumors that become resistant to EGF tyrosine kinase inhibitors increase c-Met expression, amplify c-Met, or increase c-Met only known ligand, HGF, suggesting the existence of a compensatory pathway for EGFR through c-Met. c-Met amplification was first identified in cultured cells that became resistant to gefitinib, an EGFR kinase inhibitor, and exhibited enhanced survival through the Her3 pathway. This was further validated in clinical samples where nine of 43 patients with acquired resistance to either erlotinib or gefitinib exhibited c-Met amplification.

Current small molecule and large molecule therapeutic approaches to antagonize EGFR and/or c-Met signaling pathways for therapy may be sub-optimal due to possible lack of specificity, potential off-target activity and dose-limiting toxicity that may be encountered with small molecule inhibitors. Typical monospecific bivalent antibodies may result in clustering of membrane bound receptors and unwanted activation of the downstream signaling pathways. Monovalent antibodies having full length heavy chains (half arms) pose significant complexity and cost to the manufacturing process.

Binding of a ligand such as EGF to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Inhibition of EGFR signaling may result in inhibition in one or more EGFR. In some embodiments, the EGFR ligands include EGF, TGFa, heparin binding EGF (HB-EGF), amphiregulin (AR), and epiregulm (EPI).

Binding of HGF to cMet stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division, inhibition of c-Met signaling may result in inhibition of one or more c-Met downstream signaling pathways and therefore neutralizing c-Met may have various effects, including inhibition of cell proliferation and differentiation, angiogenesis, cell motility and metastasis.

The roles of EGFR and c-Met in cancer are described, e.g., WO2014081954A1, WO2008/127710, WO2009/111691, WO2009/126834, WO2010/039248, WO2010/115551 and US2009/0042906; Engelman et al. “MET amplification leads to gefitinib resistance in lung cancer by activating ERBB3 signaling.” science 316.5827 (2007): 1039-1043; Bean et al. “MET amplification occurs with or without T790M mutations in EGFR mutant lung tumors with acquired resistance to gefitinib or erlotinib.” Proceedings of the National Academy of Sciences 104.52 (2007): 20932-20937, which are incorporated herein by reference in the entirety.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-cMET antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for cMET-1H1 (or 1H1), and cMET-1H1 derived antibodies (e.g., humanized antibodies) include CDRs of the VHH domain as set forth in SEQ ID NOs: 1, 2, and 3, respectively. The amino acid sequences for the VHH domain of cMET-1H1 antibodies are set forth in SEQ ID NOs: 19, 77, 78, 79, 80, 81, and 84.

The CDR sequences for cMET-1A12 (or 1A12), and cMET-1A12 derived antibodies (e.g., humanized antibodies) include CDRs of the VHH domain as set forth in SEQ ID NOs: 4, 5, and 6, respectively. The amino acid sequence for the VHH domain of cMET-1A12 antibody is set forth in SEQ ID NO: 20.

The CDR sequences for cMET-1E9 (or 1E9), and cMET-1E9 derived antibodies (e.g., humanized antibodies) include CDRs of the VHH domain as set forth in SEQ ID NOs: 7, 8, and 9, respectively. The amino acid sequence for the VHH domain of cMET-1E9 antibody is set forth in SEQ ID NO: 21.

The CDR sequences for cMET-1F3 (or 1F3), and cMET-1F3 derived antibodies (e.g., humanized antibodies) include CDRs of the VHH domain as set forth in SEQ ID NOs: 10, 11, and 12, respectively. The amino acid sequences for the VHH domain of cMET-1F3 antibodies are set forth in SEQ ID NOs: 22, 82, and 83.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 19-22 and 77-84.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, and SEQ ID NOs: 10-12.

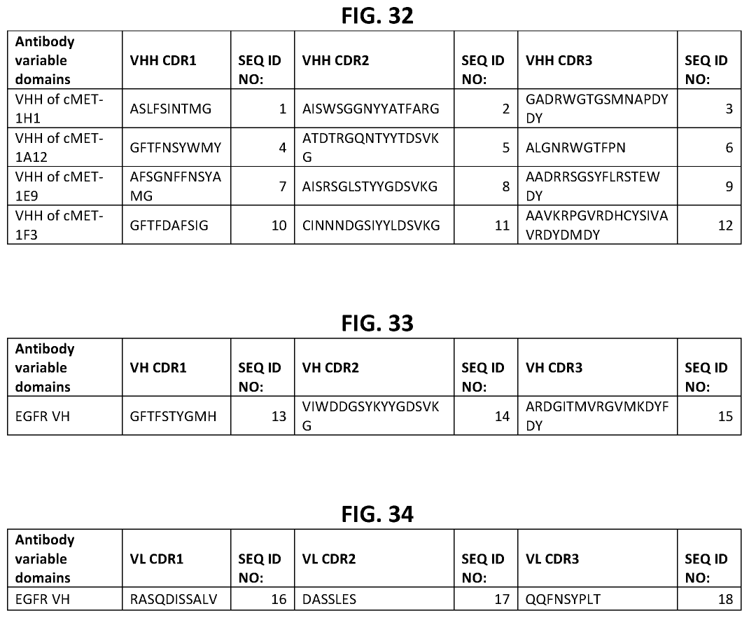

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 32.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 32.

In some embodiments, the antibody or an antigen-binding fragment described herein can contain a heavy-chain antibody variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to cMET. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 19. In some embodiments, the selected VHH sequence is SEQ ID NO: 20. In some embodiments, the selected VHH sequence is SEQ ID NO: 21. In some embodiments, the selected VHH sequence is SEQ ID NO: 22. In some embodiments, the selected VHH sequence is SEQ ID NO: 77. In some embodiments, the selected VHH sequence is SEQ ID NO: 78. In some embodiments, the selected VHH sequence is SEQ ID NO: 79. In some embodiments, the selected VHH sequence is SEQ ID NO:80. In some embodiments, the selected VHH sequence is SEQ ID NO: 81. In some embodiments, the selected VHH sequence is SEQ ID NO: 82. In some embodiments, the selected VHH sequence is SEQ ID NO: 83. In some embodiments, the selected VHH sequence is SEQ ID NO: 84.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

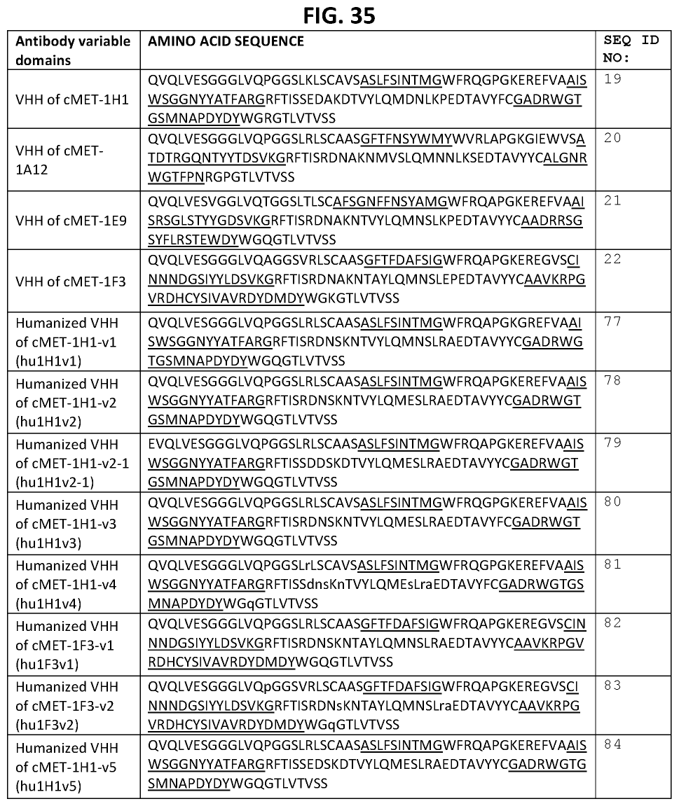

The disclosure also provides nucleic acid comprising a polynucleotide encoding a polypeptide comprising an immunoglobulin heavy-chain antibody variable domain (VHH). The VHH comprises CDRs as shown in FIG. 32, or has sequences as shown in FIG. 35.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The disclosure also provides antibodies or antigen-binding fragments thereof that bind to cMET. In some embodiments, the antibody or antigen-binding fragment thereof comprises a heavy-chain antibody variable domain (VHH) CDR1 selected from SEQ ID NO: 1, 4, 7, or 10. In some embodiments, the antibody or antigen-binding fragment thereof comprises a heavy-chain antibody variable domain (VHH) CDR2 selected from SEQ ID NO: 2, 5, 8, or 11. In some embodiments, the antibody or antigen-binding fragment thereof comprises a heavy-chain antibody variable domain (VHH) CDR3 selected from SEQ ID NO: 3, 6, 9, or 12.

### Anti-EGFR Antibodies and Antigen-Binding Fragments

The disclosure provides antibodies and antigen-binding fragments thereof that specifically bind to EGFR. The antibodies and antigen-binding fragments described herein are capable of binding to EGFR. These antibodies can be agonists or antagonists. In some embodiments, these antibodies can inhibit EGFR-associated signaling pathway (e.g., blocking binding between EGF and EGFR) thus treating cancer (e.g., NSCLC). In some embodiments, these antibodies can initiate CMC or ADCC.

The disclosure provides e.g., anti-EGFR antibody, the chimeric antibodies thereof, and the humanized antibodies thereof. In some embodiments, the CDR sequences for the anti-EGFR antibody, and its derived antibodies (e.g., humanized antibodies) include CDRs of the heavy chain variable domain, SEQ ID NOs: 13, 14, and 15. The VH with these VH CDRs can be paired with VLs with various different VL CDRs. In some embodiments, the CDR sequences for the anti-EGFR antibody, and its derived antibodies (e.g., humanized antibodies) include CDRs of the light chain variable domain, SEQ ID NOs: 16, 17, and 18.

The amino acid sequences for heavy chain variable regions and light variable regions of the humanized antibodies are also provided. As there are different ways to humanize a mouse antibody (e.g., a sequence can be modified with different amino acid substitutions), the heavy chain and the light chain of an antibody can have more than one version of humanized sequences. In some embodiments, the humanized heavy chain variable region (VH) is at least 80%, 85%, 90%, 91%, 92%, 93%, 94%, 95%, 96%, 97%, 98%, or 99% identical to SEQ ID NO: 23. In some embodiments, the humanized light chain variable region (VL) is at least 80%, 85%, 90%, 91%, 92%, 93%, 94%, 95%, 96%, 97%, 98%, or 99% identical to SEQ ID NO: 24.

The amino acid sequence for the heavy chain variable region of the anti-EGFR antibody is set forth in SEQ ID NO: 23. The amino acid sequence for the light chain variable regions of the anti-EGFR antibody is set forth in SEQ ID NO: 24.

Furthermore, in some embodiments, the antibodies or antigen-binding fragments thereof described herein can also contain one, two, or three heavy chain variable region CDRs selected from the group of SEQ ID NOs: 13-15; and/or one, two, or three light chain variable region CDRs selected from the group of SEQ ID NOs: 16-18.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 33 (VH CDR) and FIG. 34 (VL CDR).

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to EGFR. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 23, and the selected VL sequence is SEQ ID NO: 24.

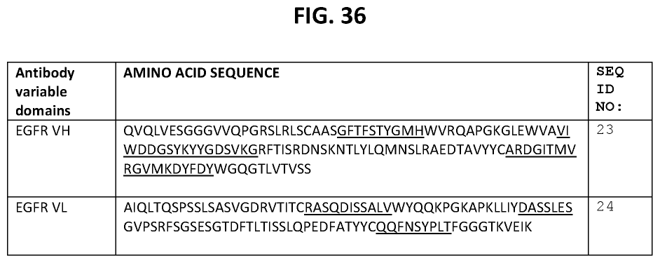

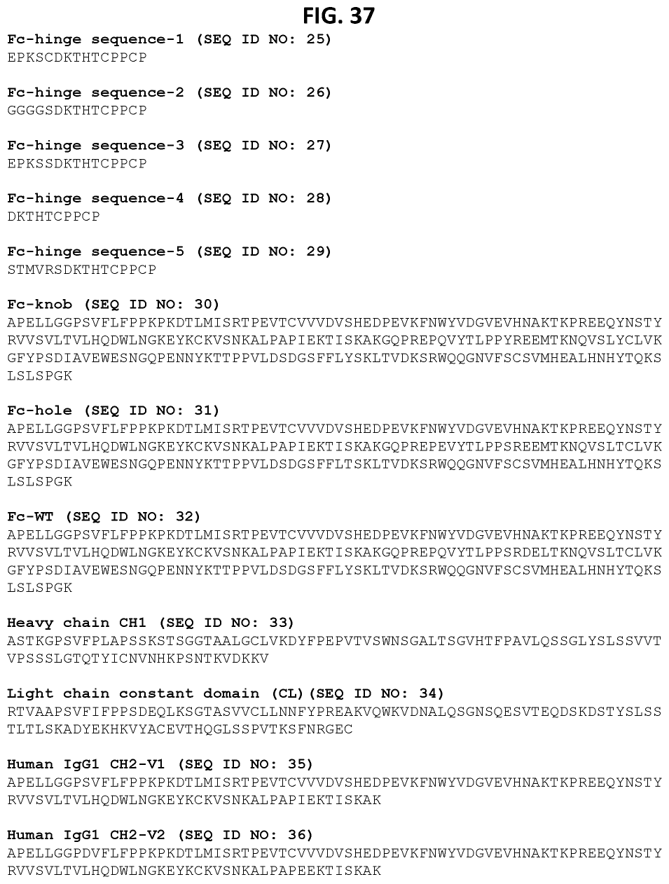

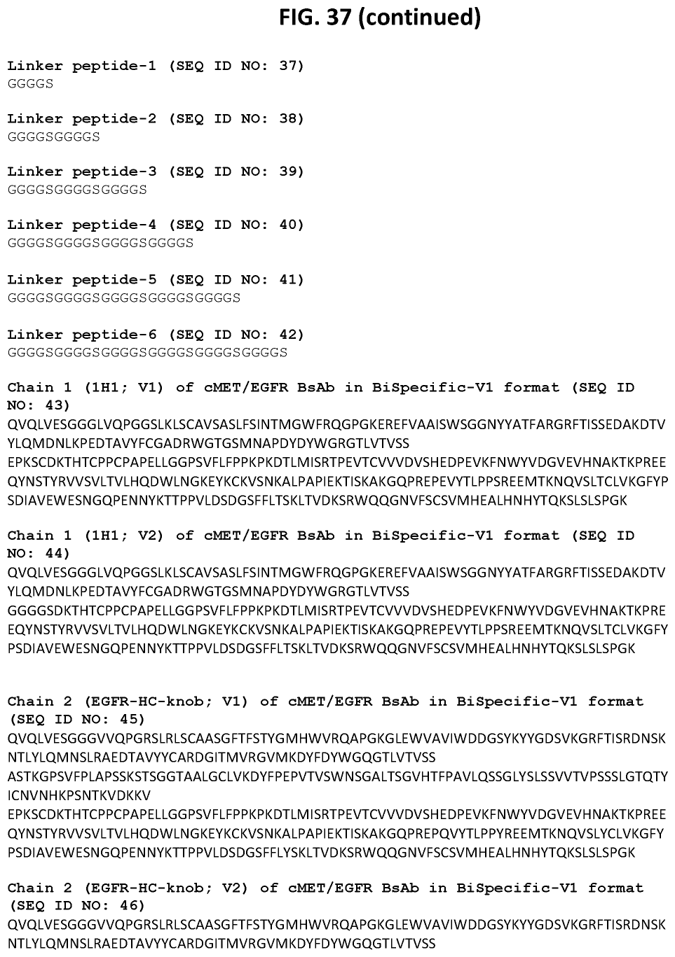

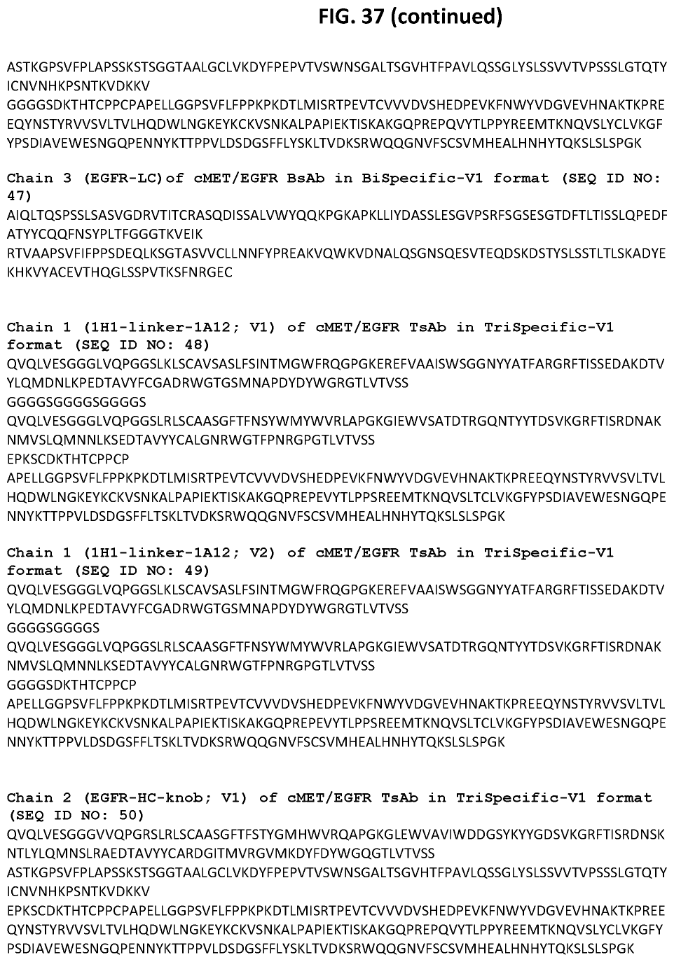

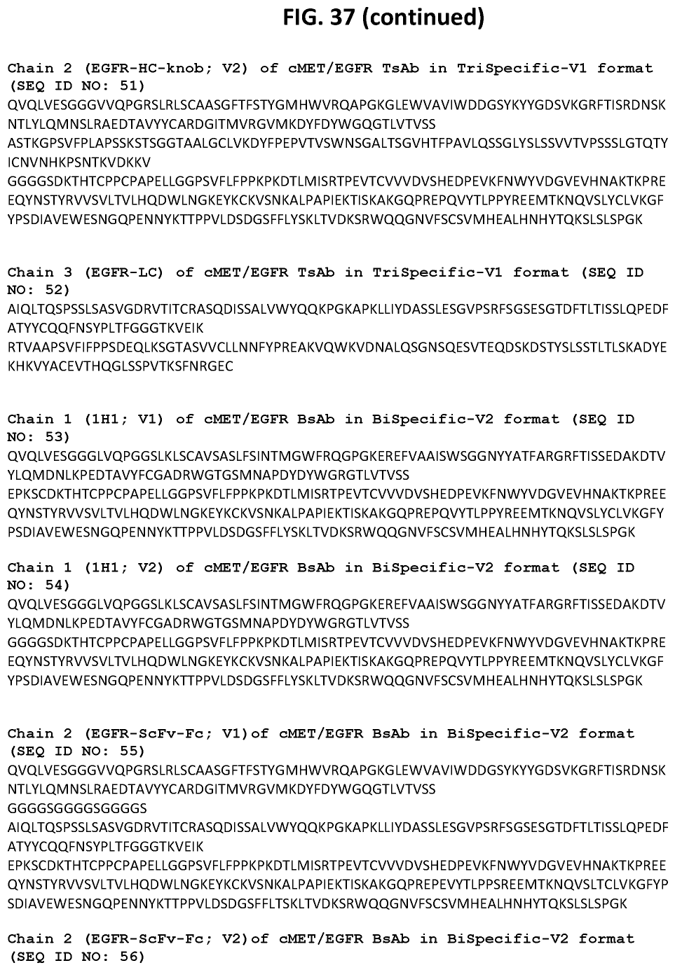

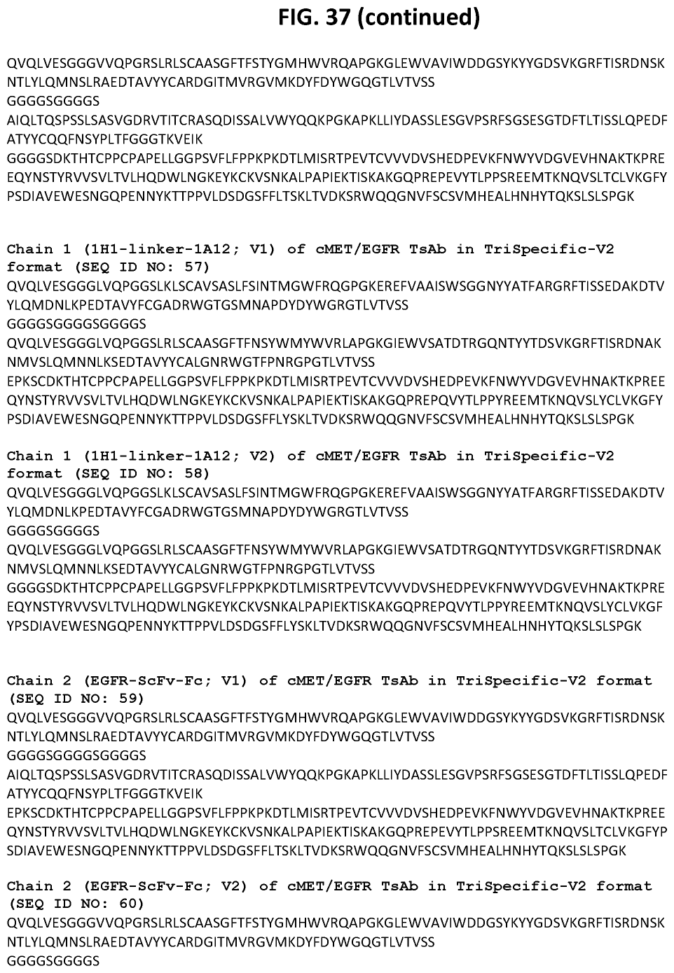

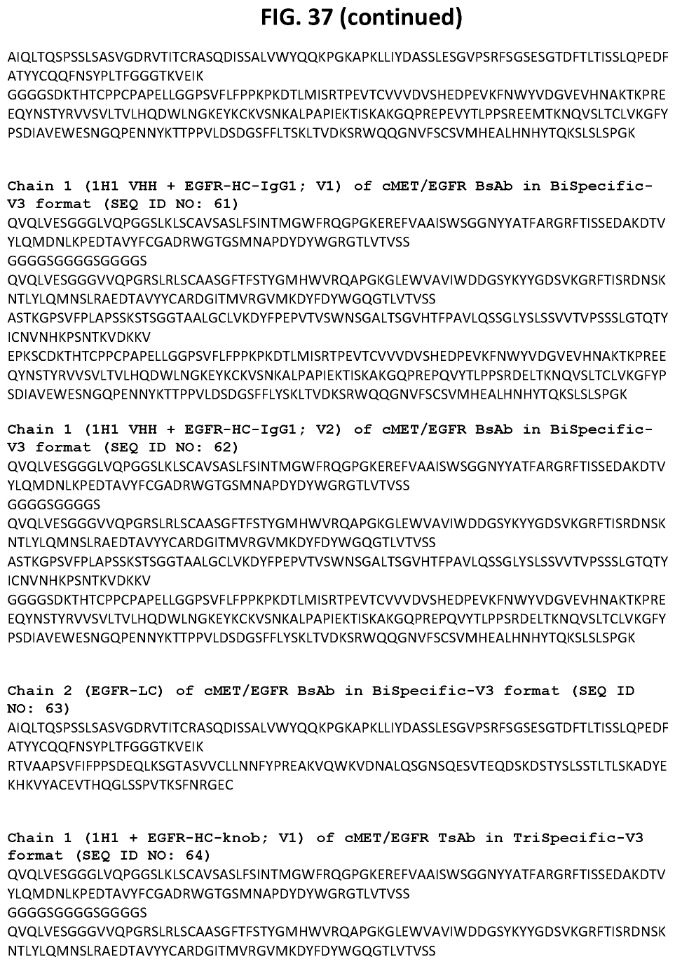

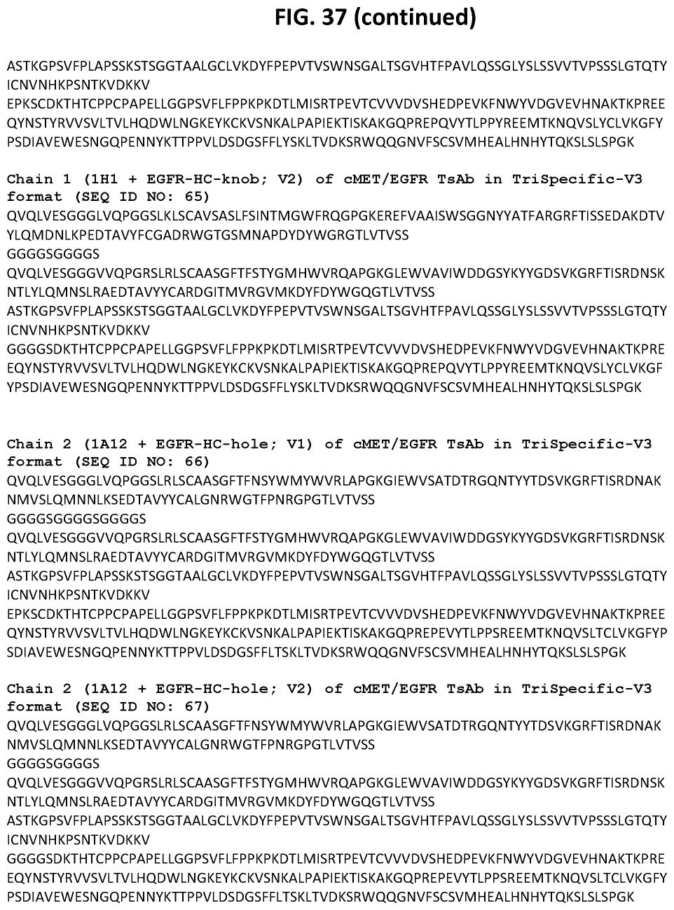

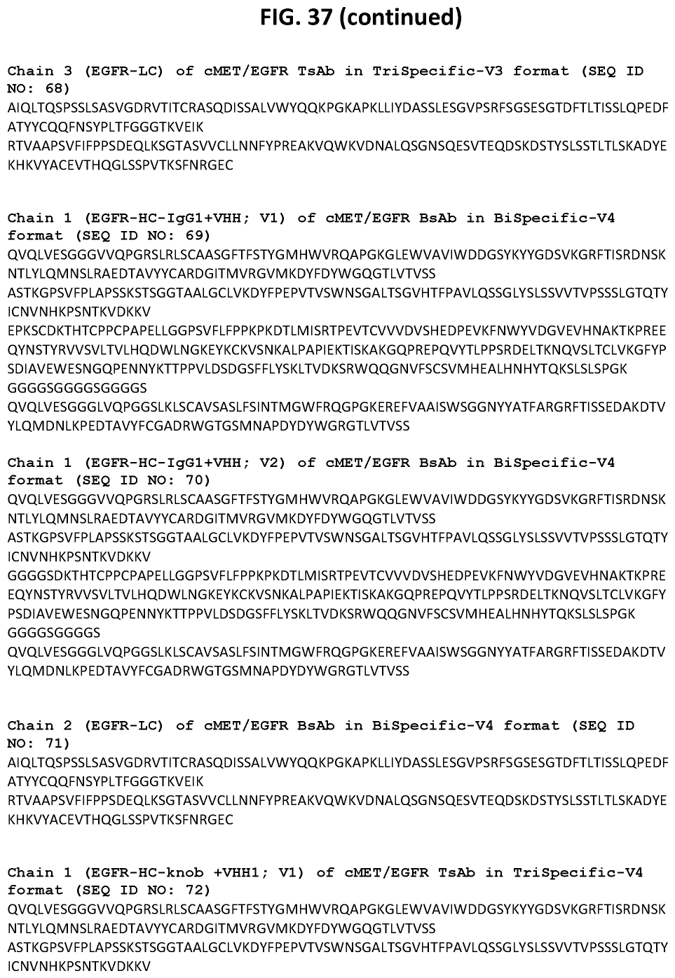

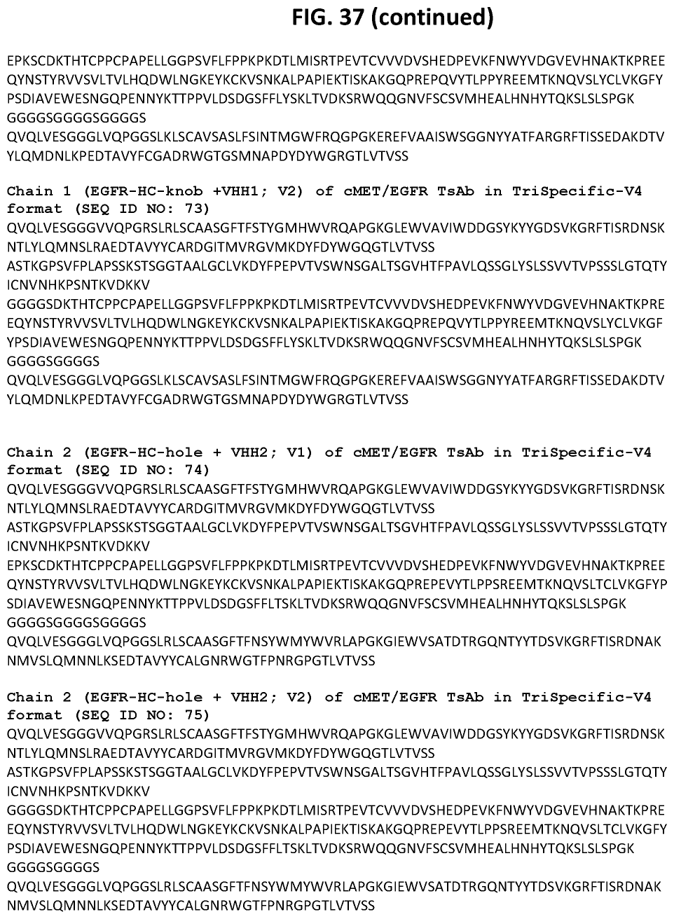

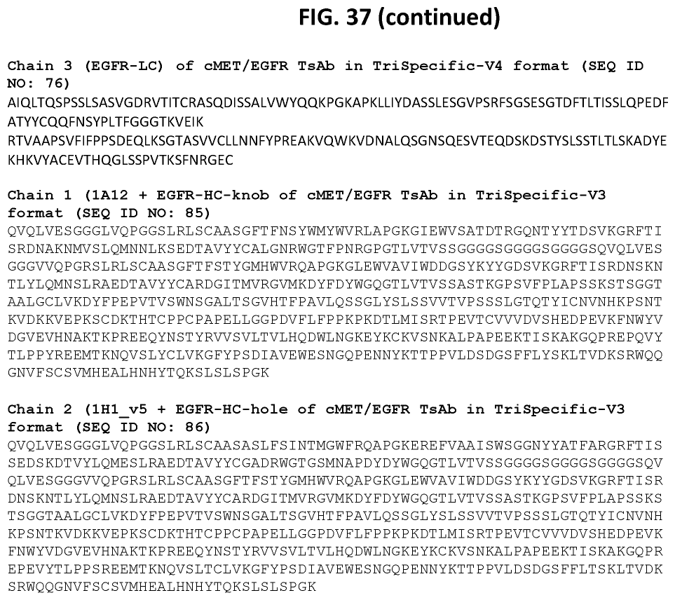

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 33 and FIG. 34, respectively, or have sequences as shown in FIG. 36. When the polypeptides are paired with corresponding polypeptide (e.g., a corresponding heavy chain variable region or a corresponding light chain variable region), the paired polypeptides bind to EGFR (e.g., human EGFR).

The anti-EGFR antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to EGFR will retain an ability to bind to EGFR.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 24 binds to EGFR; or
(2) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 23 binds to EGFR.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the VH when paired with a VL specifically binds to human EGFR, or the VL when paired with a VH specifically binds to human EGFR.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, wherein the vector encodes the VL region and the VH region that together bind to EGFR. In one aspect, the disclosure is related to a pair of vectors, wherein each vector comprises one of the nucleic acids described herein, wherein together the pair of vectors encodes the VL region and the VH region that together bind to EGFR.

In one aspect, the disclosure is related to a cell comprising the vector or the pair of vectors described herein. In one aspect, the disclosure is related to two of the nucleic acids described herein.

In some embodiments, the two nucleic acids together encode the VL region and the VH region that together bind to EGFR.

In one aspect, the disclosure is related to an antibody or antigen-binding fragment thereof that binds to EGFR (epidermal growth factor receptor) comprising:
a heavy chain variable region (VH) comprising complementarity determining regions (CDRs) 1, 2, and 3, wherein the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15; and
a light chain variable region (VL) comprising CDRs 1, 2, and 3, wherein the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

In one aspect, the disclosure is related to an antibody or antigen-binding fragment thereof that binds to EGFR comprising a heavy chain variable region (VH) comprising an amino acid sequence that is at least 90% identical to SEQ ID NO: 23, and a light chain variable region (VL) comprising an amino acid sequence that is at least 90% identical to SEQ ID NO: 24.

In some embodiments, the VH comprises the sequence of SEQ ID NO: 23 and the VL comprises the sequence of SEQ ID NO: 24.

In some embodiments, the antibody or antigen-binding fragment specifically binds to human EGFR.

In some embodiments, the antibody or antigen-binding fragment is a single-chain variable fragment (scFV).

In one aspect, the disclosure is related to an antibody or antigen-binding fragment thereof comprising the VH CDRs 1, 2, 3, and VL CDRs 1, 2, 3, of the antibody or antigen-binding fragment thereof as described herein.

In one aspect, the disclosure is related to an antibody or antigen-binding fragment thereof that cross-competes with the antibody or antigen-binding fragment thereof as described herein.

In some embodiments, the antibody described herein is a bispecific antibody or a multispecific antibody.

Structures of cMET/EGFR Bispecific Antibodies

In some embodiments, the bispecific antibodies are designed to include a VHH that targets cMET. The bispecific antibodies are described below.

cMET (tyrosine-protein kinase Met, or hepatocyte growth factor receptor (HGFR)) is single pass tyrosine kinase receptor essential for embryonic development, organogenesis and wound healing. The present disclosure provides bispecific antibodies that bind to both cMET and EGFR. The bispecific antibodies can be used to treat cMET or EGFR positive cancers (e.g., non-small cell lung cancer) in a subject.

The cMET/EGFR bispecific antibodies with specific structures are described below.

### BiSpecific-V1 Structure

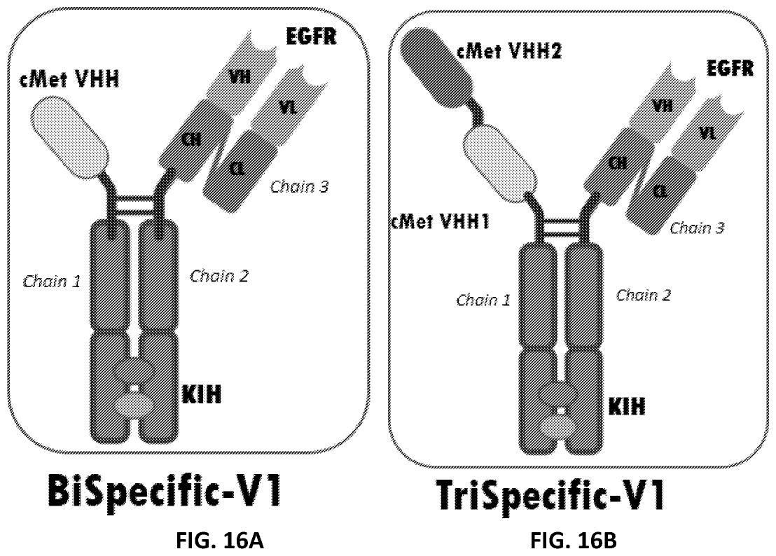

As shown in FIG. 16A, a cMET/EGFR bispecific antibody can be prepared to have a BiSpecific-V1 structure. Specifically, the cMET/EGFR bispecific antibody comprises (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to cMET. In some embodiments, the VH and the VL associate with each other and specifically bind to EGFR.

In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the cMET/EGFR bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 43 or 44. In some embodiments, the second polypeptide comprises a sequence that is at least 80%, 85%, 90%, 95%, or 100% identical to SEQ ID NO: 45 or 46. In some embodiments, the third polypeptide comprises a sequence that is at least 80%, 85%, 90%, 95%, or 100% identical to SEQ ID NO: 47.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the second polypeptide comprise a sequence that is at least 80%, 85%, 90%, 95%, or 100% identical to SEQ ID NO: 33. In some embodiments, the third polypeptide comprises a sequence that is at least 80%, 85%, 90%, 95%, or 100% identical to SEQ ID NO: 34.

### BiSpecific-V2 Structure

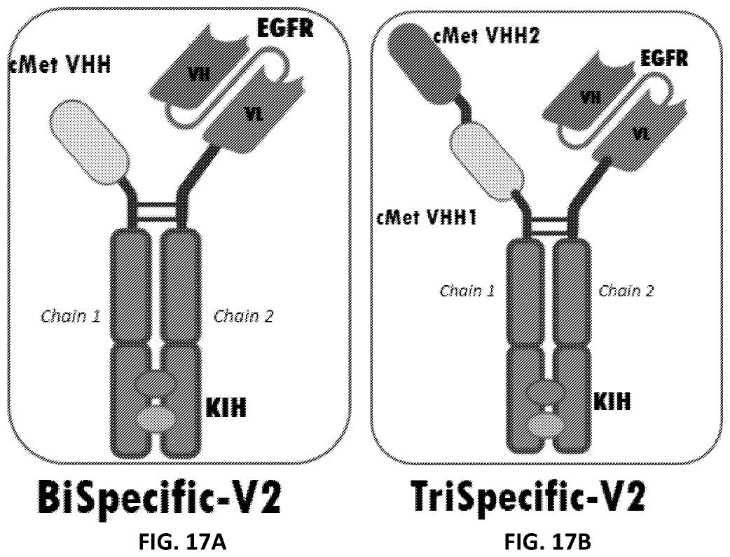

As shown in FIG. 17A, a cMET/EGFR bispecific antibody can be prepared to have a BiSpecific-V2 structure. Specifically, the cMET/EGFR bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region.

In some embodiments, the first VHH specifically binds to cMET. In some embodiments, the scFv comprises a heavy chain variable region (VH), a linker peptide sequence, and a light chain variable region (VL). In some embodiments, the VH and the VL associate with each other and specifically bind to EGFR.

In some embodiments, the linker peptide sequence comprises a sequence that is at least 80%, 85%, 90%, 95%, or 100% identical to any one SEQ ID NOs: 37-42. In some embodiments, the linker peptide sequence comprises a sequence that is at least 80%, 85%, 90%, 95%, or 100% identical to SEQ ID NO: 38 or 39. In some embodiments, the linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37). In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the cMET/EGFR bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 53 or 54. In some embodiments, the second polypeptide comprises a sequence that is at least 80%, 85%, 90%, 95%, or 100% identical to SEQ ID NO: 55 or 56.

In some embodiments, the first polypeptide comprises a sequence that is at least 80%, 85%, 90%, 95%, or 100% identical to SEQ ID NO: 57 or 58. In some embodiments, the second polypeptide comprises a sequence that is at least 80% identical to SEQ ID NO: 59 or 60.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

### BiSpecific-V3 Structure

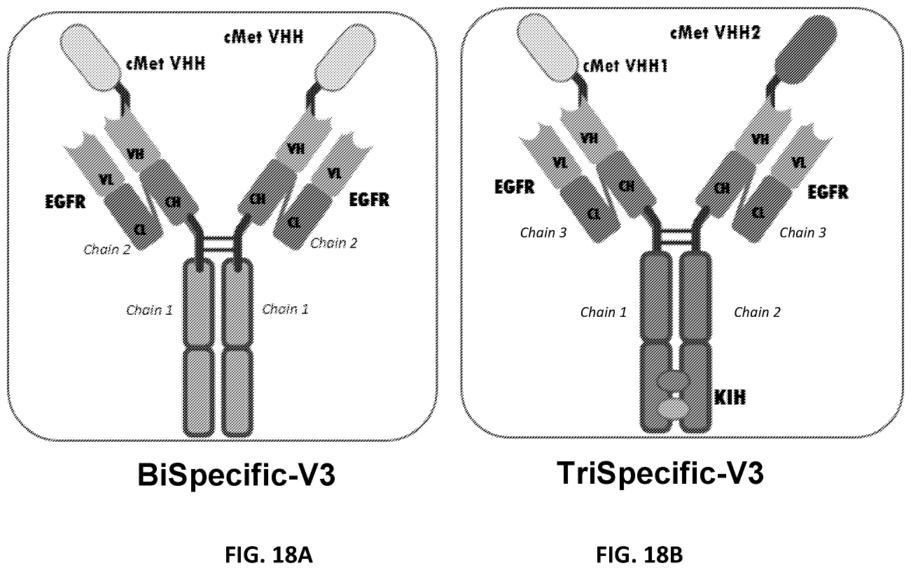

As shown in FIG. 18A, a cMET/EGFR bispecific antibody can be prepared to have a BiSpecific-V3 structure. Specifically, the cMET/EGFR bispecific antibody comprises (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80%, 85%, 90%, 95%, or 100% identical to any one SEQ ID NOs: 37-42. In some embodiments, the first linker peptide sequence and/or the second linker peptide sequence comprises a sequence that is at least 80% identical to SEQ ID NO: 38 or 39. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 32. In some embodiments, the first polypeptide comprises a sequence that is at least 80%, 85%, 90%, 95%, or 100% identical to SEQ ID NO: 61 or 62. In some embodiments, the second polypeptide comprises a sequence that is at least 80%, 85%, 90%, 95%, or 100% identical to SEQ ID NO: 63. In some embodiments, the third polypeptide comprises a sequence that is at least 80%, 85%, 90%, 95%, or 100% identical to SEQ ID NO: 61 or 62. In some embodiments, the fourth polypeptide comprises a sequence that is at least 80%, 85%, 90%, 95%, or 100% identical to SEQ ID NO: 63.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the first polypeptide and/or the third polypeptide comprise a sequence that is at least 80%, 85%, 90%, 95%, or 100% identical to SEQ ID NO: 33. In some embodiments, the second polypeptide and/or the fourth polypeptide comprise a sequence that is at least 80%, 85%, 90%, 95%, or 100% identical to SEQ ID NO: 34.

### BiSpecific-V4 Structure

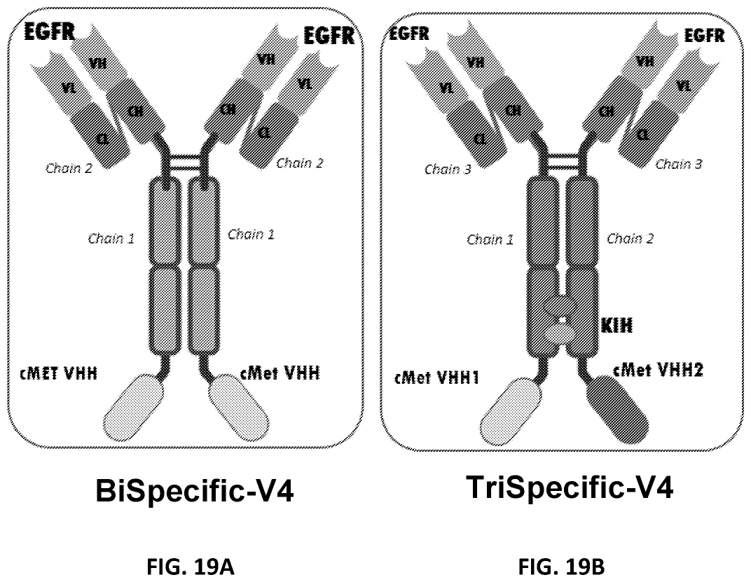

As shown in FIG. 19A, a cMET/EGFR bispecific antibody can be prepared to have a BiSpecific-V4 structure. Specifically, the cMET/EGFR bispecific antibody comprises (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80%, 85%, 90%, 95%, or 100% identical to any one SEQ ID NOs: 37-42. In some embodiments, the first linker peptide sequence and/or the second linker peptide sequence comprises a sequence that is at least 80%, 85%, 90%, 95%, or 100% identical to SEQ ID NO: 38 or 39. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 32. In some embodiments, the first polypeptide comprises a sequence that is at least 80%, 85%, 90%, 95%, or 100% identical to SEQ ID NO: 69 or 70. In some embodiments, the second polypeptide comprises a sequence that is at least 80%, 85%, 90%, 95%, or 100% identical to SEQ ID NO: 71. In some embodiments, the third polypeptide comprises a sequence that is at least 80%, 85%, 90%, 95%, or 100% identical to SEQ ID NO: 69 or 70. In some embodiments, the fourth polypeptide comprises a sequence that is at least 80%, 85%, 90%, 95%, or 100% identical to SEQ ID NO: 71.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the first polypeptide and/or the third polypeptide comprise a sequence that is at least 80%, 85%, 90%, 95%, or 100% identical to SEQ ID NO: 33. In some embodiments, the second polypeptide and/or the fourth polypeptide comprise a sequence that is at least 80%, 85%, 90%, 95%, or 100% identical to SEQ ID NO: 34.

In some embodiments, the VHH that specifically binds to cMET of the cMET/EGFR bispecific antibody with the BiSpecific-V1, BiSpecific-V2, BiSpecific-V3, or BiSpecific-V4 structure can be selected from any of the VHHs targeting cMET described in the disclosure.

In some embodiments, the VH, when associated with a VL, that specifically binds to EGFR of the cMET/EGFR bispecific antibody with the BiSpecific-V1, BiSpecific-V2, BiSpecific-V3, or BiSpecific-V4 structure can be selected from any of the VH targeting EGFR described in the disclosure.

In some embodiments, the VL, when associated with a VH, that specifically binds to EGFR of the cMET/EGFR bispecific antibody with the BiSpecific-V1, BiSpecific-V2, BiSpecific-V3, or BiSpecific-V4 structure can be selected from any of the VL targeting EGFR described in the disclosure.

Structures of cMET/EGFR Multispecific Antibodies

In some embodiments, the multi-specific (e.g., trispecific) antibodies are designed to include a VHH that targets cMET. In some embodiments, the multi-specific (e.g., trispecific) antibodies are designed that include an additional VHH that targets cMET. The multispecific (e.g., trispecific) antibodies are described below.

cMET (tyrosine-protein kinase Met, or hepatocyte growth factor receptor (HGFR)) is single pass tyrosine kinase receptor essential for embryonic development, organogenesis and wound healing. The present disclosure provides multi-specific (e.g., trispecific) antibodies that bind to both cMET and EGFR. The trispecific antibodies can be used to treat cMET or EGFR positive cancers (e.g., non-small cell lung cancer) in a subject.

The cMET/EGFR trispecific antibodies with specific structures are described below.

### TriSpecific-V1 Structures

As shown in FIG. 16B, a cMET/EGFR trispecific antibody can be prepared to have a TriSpecific-V1 structure. Specifically, the cMET/EGFR trispecific antibody comprises (a) a first polypeptide comprising from N-terminus to C-terminus: a second heavy-chain antibody variable domain (VHH), a linker peptide sequence, a first VHH, a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH and the second VHH specifically bind to cMET. In some embodiments, the VH and the VL associate with each other and specifically bind to EGFR.

In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the linker peptide sequence comprises a sequence that is at least 80%, 85%, 90%, 95%, or 100% identical to any one SEQ ID NOs: 37-42. In some embodiments, the linker peptide sequence comprises a sequence that is at least 80%, 85%, 90%, 95%, or 100% identical to SEQ ID NO: 38 or 39. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first polypeptide comprises a sequence that is at least 80%, 85%, 90%, 95%, or 100% identical to SEQ ID NO: 48 or 49. In some embodiments, the second polypeptide comprises a sequence that is at least 80%, 85%, 90%, 95%, or 100% identical to SEQ ID NO: 50 or 51. In some embodiments, the third polypeptide comprises a sequence that is at least 80%, 85%, 90%, 95%, or 100% identical to SEQ ID NO: 52.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the second polypeptide comprise a sequence that is at least 80%, 85%, 90%, 95%, or 100% identical to SEQ ID NO: 33. In some embodiments, the third polypeptide comprises a sequence that is at least 80%, 85%, 90%, 95%, or 100% identical to SEQ ID NO: 34.

### TriSpecific-V2 Structures

As shown in FIG. 17B, a cMET/EGFR trispecific antibody can be prepared to have a TriSpecific-V2 structure. Specifically, the cMET/EGFR trispecific antibody comprises (a) a first polypeptide comprising from N-terminus to C-terminus: a second heavy-chain antibody variable domain (VHH), a second linker peptide sequence, a first VHH, a first hinge region, a first Fc region; and (b) a second polypeptide comprising from N-terminus to C-terminus: a single-chain variable fragment (scFv), a second hinge region, and a second Fc region.

In some embodiments, the first and the second VHH specifically binds to cMET. In some embodiments, the scFv comprises a heavy chain variable region (VH), a first linker peptide sequence, and a light chain variable region (VL). In some embodiments, the VH and the VL associate with each other and specifically bind to EGFR.

In some embodiments, the first hinge region and/or the second hinge region comprise a sequence that is at least 80%, 85%, 90%, 95%, or 100% identical to any one of SEQ ID NOs: 25-29. In some embodiments, the first Fc region and/or the second Fc region comprise a sequence that is at least 80%, 85%, 90%, 95%, or 100% identical to SEQ ID NO: 30 or 31.

In some embodiments, the cMET/EGFR trispecific antibody comprises knob-into-hole mutations. In some embodiments, the Fc region is an IgG1 Fc region.

In some embodiments, the first linker peptide sequence and/or the second linker peptide sequence comprise a sequence that is at least 80%, 85%, 90%, 95%, or 100% identical to any one SEQ ID NOs: 37-42. In some embodiments, the first linker peptide sequence and/or the second linker peptide sequence comprise a sequence that is at least 80%, 85%, 90%, 95%, or 100% identical to SEQ ID NO: 38 or 39. In some embodiments, the first linker peptide sequence and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first polypeptide comprises a sequence that is at least 80%, 85%, 90%, 95%, or 100% identical to SEQ ID NO: 57 or 58. In some embodiments, the second polypeptide comprises a sequence that is at least 80% identical to SEQ ID NO: 59 or 60.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

### TriSpecific-V3 Structures

As shown in FIG. 18B, a cMET/EGFR trispecific antibody can be prepared to have a TriSpecific-V3 structure. Specifically, the cMET/EGFR trispecific antibody comprises (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80%, 85%, 90%, 95%, or 100% identical to any one SEQ ID NOs: 37-42. In some embodiments, the first linker peptide sequence and/or the second linker peptide sequence comprises a sequence that is at least 80% identical to SEQ ID NO: 38 or 39. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 as described in the TriSpecific-V3 structure are different. In some embodiments, the first Fc region and/or the second Fc region comprise a sequence that is at least 80%, 85%, 90%, 95%, or 100% identical to SEQ ID NO: 30 or 31. In some embodiments, the first polypeptide comprises a sequence that is at least 80%, 85%, 90%, 95%, or 100% identical to SEQ ID NO: 64 or 65. In some embodiments, the second polypeptide comprises a sequence that is at least 80%, 85%, 90%, 95%, or 100% identical to SEQ ID NO: 68. In some embodiments, the third polypeptide comprises a sequence that is at least 80%, 85%, 90%, 95%, or 100% identical to SEQ ID NO: 66 or 67. In some embodiments, the fourth polypeptide comprises a sequence that is at least 80%, 85%, 90%, 95%, or 100% identical to SEQ ID NO: 68.

In some embodiments, the first polypeptide comprises a sequence that is at least 80%, 85%, 90%, 95%, or 100% identical to SEQ ID NO: 85. In some embodiments, the second polypeptide comprises a sequence that is at least 80%, 85%, 90%, 95%, or 100% identical to SEQ ID NO: 68. In some embodiments, the third polypeptide comprises a sequence that is at least 80%, 85%, 90%, 95%, or 100% identical to SEQ ID NO: 86. In some embodiments, the fourth polypeptide comprises a sequence that is at least 80%, 85%, 90%, 95%, or 100% identical to SEQ ID NO: 68.

In some embodiments, the Fc region is an IgG1 (e.g., human IgG1) Fc region. In some embodiments, the cMET/EGFR trispecific antibody described herein comprises knob-into-hole mutations.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the first polypeptide and/or the third polypeptide comprise a sequence that is at least 80%, 85%, 90%, 95%, or 100% identical to SEQ ID NO: 33. In some embodiments, the second polypeptide and/or the fourth polypeptide comprise a sequence that is at least 80%, 85%, 90%, 95%, or 100% identical to SEQ ID NO: 34.

### TriSpecific-V4 Structures

As shown in FIG. 19B, a cMET/EGFR trispecific antibody can be prepared to have a TriSpecific-V4 structure. Specifically, the cMET/EGFR trispecific antibody comprises (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to cMET. In some embodiments, the VH1 and the VL1 associate with each other and specifically bind to EGFR. In some embodiments, the VH2 and the VL2 associate with each other and specifically bind to EGFR.

In some embodiments, the first linker peptide sequence and/or the second linker peptide sequence comprises a sequence that is at least 80%, 85%, 90%, 95%, or 100% identical to any one SEQ ID NOs: 37-42. In some embodiments, the first linker peptide sequence and/or the second linker peptide sequence comprises a sequence that is at least 80%, 85%, 90%, 95%, or 100% identical to SEQ ID NO: 38 or 39. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 37).

In some embodiments, the first hinge region and/or the second hinge regions comprise a sequence that is at least 80%, 85%, 90%, 95%, or 100% identical to any one of SEQ ID NOs: 25-29.

In some embodiments, sequences of the VHH1 and the VHH2 as described in the TriSpecific-V4 structure are different. In some embodiments, the first Fc region and/or the second Fc region comprise a sequence that is at least 80%, 85%, 90%, 95%, or 100% identical to SEQ ID NO: 30 or 31. In some embodiments, the first polypeptide comprises a sequence that is at least 80%, 85%, 90%, 95%, or 100% identical to SEQ ID NO: 72 or 73. In some embodiments, the second polypeptide comprises a sequence that is at least 80%, 85%, 90%, 95%, or 100% identical to SEQ ID NO: 76. In some embodiments, the third polypeptide comprises a sequence that is at least 80%, 85%, 90%, 95%, or 100% identical to SEQ ID NO: 74 or 75. In some embodiments, the fourth polypeptide comprises a sequence that is at least 80%, 85%, 90%, 95%, or 100% identical to SEQ ID NO: 76.

In some embodiments, the Fc region is an IgG1 (e.g., human IgG1) Fc region. In some embodiments, the cMET/EGFR trispecific antibody described herein comprises knob-into-hole mutations.

In some embodiments, the first Fc region and/or the second Fc region comprises an aspartic acid (Asp) at position 239 according to EU numbering. In some embodiments, the first Fc region and/or the second Fc region comprises a glutamic acid (Glu) at position 332 according to EU numbering. In some embodiments, the first Fc region and/or the second Fc region comprises a sequence that is at least 80%, 85%, 90%, 95%, or 100% identical to SEQ ID NO: 35 or 36.

In some embodiments, the first polypeptide and/or the third polypeptide comprise a sequence that is at least 80%, 85%, 90%, 95%, or 100% identical to SEQ ID NO: 33. In some embodiments, the second polypeptide and/or the fourth polypeptide comprise a sequence that is at least 80%, 85%, 90%, 95%, or 100% identical to SEQ ID NO: 34.

In some embodiments, the VHH that specifically binds to cMET of the cMET/EGFR trispecific antibody with the TriSpecific-V1, TriSpecific-V2, TriSpecific-V3, or TriSpecific-V4 structure can be selected from any of the VHHs targeting cMET described in the disclosure.

In some embodiments, the VH, when associated with a VL, that specifically binds to EGFR of the cMET/EGFR trispecific antibody with the TriSpecific-V1, TriSpecific-V2, TriSpecific-V3, or TriSpecific-V4 structure can be selected from any of the VH targeting EGFR described in the disclosure.

In some embodiments, the VL, when associated with a VH, that specifically binds to EGFR of the cMET/EGFR trispecific antibody with the TriSpecific-V1, TriSpecific-V2, TriSpecific-V3, or TriSpecific-V4 structure can be selected from any of the VL targeting EGFR described in the disclosure.

### Antibody Characteristics

The anti-cMET, anti-EGFR, or anti-cMET/EGFR antigen-binding protein construct (e.g., antibodies, bispecific antibodies, trispecific antibodies, multi-specific antibodies, or antibody fragments thereof) can include an antigen binding site that is derived from any anti-cMET antibody, anti-EGFR antibody, or any antigen-binding fragment thereof as described herein.

In some embodiments, the antibodies or antigen-binding fragments thereof described herein are EGFR antagonist. In some embodiments, the antibodies or antigen-binding fragments thereof are EGFR agonist. In some embodiments, the antibodies or antigen-binding fragments thereof as described herein are cMET antagonist. In some embodiments, the antibodies or antigen-binding fragments thereof are cMET agonist.

In some embodiments, the antibodies, or antigen-binding fragments thereof described herein can bind to cMET and/or EGFR, thereby blocking the interaction of these receptors and their respective ligands; decreasing the phosphorylation of cMET and/or EGFR; decreasing the phosphorylation of downstream signaling pathways (e.g., ERK and/or Akt pathways); and/or directly killing the cancer cells by ADCC and/or CDC.

In some embodiments, the antibodies or antigen-binding fragments thereof described herein has a cMET binding capability (e.g., determined by ELISA) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of JNJ-372 or JNJ-372 analog.

In some embodiments, the antibodies or antigen-binding fragments thereof described herein has a EGFR binding capability (e.g., determined by ELISA) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of JNJ-372 or JNJ-372 analog.

In some embodiments, the EC50 of the antibodies or antigen-binding fragments thereof described herein for binding to cMET is less than or about 50 ng/ml, less than or about 40 ng/ml, less than or about 30 ng/ml, less than or about 20 ng/ml, less than or about 15 ng/ml, less than or about 10 ng/ml, less than or about 9 ng/ml, less than or about 8 ng/ml, less than or about 7 ng/ml, less than or about 6 ng/ml, less than or about 5 ng/ml, less than or about 4 ng/ml, less than or about 3 ng/ml, less than or about 2 ng/ml, less than or about 1 ng/ml. In some embodiments, the EC50 of the antibodies or antigen-binding fragments thereof described herein for binding to cMET is about 1 ng/ml to about 20 ng/ml, about 1 ng/ml to about 10 ng/ml, or about 5 ng/ml to about 10 ng/ml.

In some embodiments, the EC50 of the antibodies or antigen-binding fragments thereof described herein for blocking cMET/HGF interaction is less than or about 1 nM, less than or about 0.9 nM, less than or about 0.8 nM, less than or about 0.7 nM, less than or about 0.6 nM, less than or about 0.5 nM, less than or about 0.4 nM, less than or about 0.3 nM. In some embodiments, the EC50 of the antibodies or antigen-binding fragments thereof described herein for blocking cMET/HGF interaction is about 0.1 nM to about 1 nM, about 0.1 nM to about 0.8 nM, or about 0.1 nM to about 0.6 nM. In some embodiments, the EC50 of the antibody or antigen-binding fragment thereof described herein for blocking cMET/HGF interaction is less than 80%, less than 70%, less than 60%, less than 50%, or less than 40% as compared to the EC50 of JNJ372 or JNJ372 analog using the substantially identical determination method.

In some embodiments, the EC50 of the antibodies or antigen-binding fragments thereof described herein for binding to EGFR is less than or about 50 ng/ml, less than or about 40 ng/ml, less than or about 30 ng/ml, less than or about 20 ng/ml, less than or about 15 ng/ml, less than or about 10 ng/ml, less than or about 9 ng/ml, less than or about 8 ng/ml, less than or about 7 ng/ml, less than or about 6 ng/ml, less than or about 5 ng/ml, less than or about 4 ng/ml, less than or about 3 ng/ml, less than or about 2 ng/ml, less than or about 1 ng/ml. In some embodiments, the EC50 of the antibodies or antigen-binding fragments thereof described herein for binding to EGFR is about 1 ng/ml to about 20 ng/ml, about 1 ng/ml to about 15 ng/ml, about 1 ng/ml to about 10 ng/ml, or about 5 ng/ml to about 10 ng/ml.

In some embodiments, the EC50 of the antibodies or antigen-binding fragments thereof described herein for blocking EGFR/EGF interaction is less than or about 1 nM, less than or about 0.9 nM, less than or about 0.8 nM, less than or about 0.7 nM, less than or about 0.6 nM, less than or about 0.5 nM, less than or about 0.4 nM, less than or about 0.3 nM. In some embodiments, the EC50 of the antibodies or antigen-binding fragments thereof described herein for blocking EGFR/EGF interaction is about 0.2 nM to about 1 nM, about 0.2 nM to about 0.8 nM, or about 0.2 nM to about 0.6 nM. In some embodiments, the EC50 of the antibody or antigen-binding fragment thereof described herein for blocking EGFR/EGF interaction is less than 50%, less than 40%, less than 30%, less than 20%, less than 10%, less than 5%, or less than 3% as compared to the EC50 of JNJ372 or JNJ372 analog using the substantially identical determination method.

In some embodiments, the EC50 of the antibodies or antigen-binding fragments thereof described herein for binding to NSCLC cells (e.g., any one of the NSCLC cells described herein) is less than or about 1 nM, less than or about 0.9 nM, less than or about 0.8 nM, less than or about 0.7 nM, less than or about 0.6 nM, less than or about 0.5 nM, less than or about 0.4 nM, less than or about 0.3 nM, less than or about 0.2 nM, less than or about 0.15 nM, less than or about 0.14 nM, less than or about 0.13 nM, less than or about 0.1 nM. In some embodiments, the EC50 of the antibodies or antigen-binding fragments thereof described herein for binding to NSCLC cells is about 0.1 nM to about 1 nM, about 0.1 nM to about 0.5 nM, about 0.1 nM to about 0.2 nM, or about 0.1 nM to about 0.15 nM. In some embodiments, the EC50 of the antibody or antigen-binding fragment thereof described herein for binding to NSCLC cells is less than 50%, less than 40%, less than 30%, less than 20%, less than 10%, or less than 5% as compared to the EC50 of JNJ372 or JNJ372 analog using the substantially identical determination method.

In some embodiments, the EC50 of the antibodies or antigen-binding fragments thereof described herein for inhibiting proliferation of NSCLC cell (e.g., any one of the NSCLC cells described herein) is less than or about 2 nM, less than or about 1.5 nM, less than or about 1 nM, less than or about 0.9 nM, less than or about 0.8 nM, less than or about 0.7 nM, less than or about 0.6 nM, less than or about 0.5 nM, less than or about 0.4 nM, less than or about 0.3 nM, less than or about 0.2 nM, less than or about 0.1 nM, less than or about 0.05 nM, less than or about 0.04 nM, less than or about 0.03 nM. In some embodiments, the EC50 of the antibodies or antigen-binding fragments thereof described herein for inhibiting proliferation of NSCLC cell is about 0.03 nM to about 2 nM, about 0.03 nM to about 1 nM, or about 0.03 nM to about 0.3 nM. In some embodiments, the EC50 of the antibody or antigen-binding fragment thereof described herein for binding to NSCLC cells is less than 20%, less than 10%, less than 5%, less than 3%, less than 1%, less than 0.5%, less than 0.1%, less than 0.05%, or less than 0.01% as compared to the EC50 of JNJ372 or JNJ372 analog using the substantially identical determination method.

In some embodiments, the IC50 of the antibodies or antigen-binding fragments thereof described herein for inducing internalization is less than or about 0.5 nM, less than or about 0.4 nM, less than or about 0.3 nM, less than or about 0.2 nM, less than or about 0.15 nM, less than or about 0.1 nM, less than or about 0.09 nM, less than or about 0.08 nM, less than or about 0.07 nM, less than or about 0.06 nM, less than or about 0.05 nM, less than or about 0.02 nM, less than or about 0.01 nM. In some embodiments, the IC50 of the antibodies or antigen-binding fragments thereof described herein for inducing internalization is about 0.01 nM to about 0.1 nM, about 0.01 nM to about 0.05 nM, or about 0.05 nM to about 0.1 nM. In some embodiments, the IC50 of the antibody or antigen-binding fragment thereof described herein for inducing internalization is less than 70%, less than 50%, less than 40%, less than 30%, less than 20%, less than 10%, less than 5%, or less than 3% as compared to the IC50 of JNJ372 or JNJ372 analog using the substantially identical determination method.

In some embodiments, the antibodies or antigen-binding fragments thereof described herein has a better effector function (e.g., ADCC effect) than JNJ372 or JNJ372 analog in NSCLC cells (e.g., H1975 cells).

In some embodiments, the antibodies or antigen-binding fragments thereof described herein has a better cancer cell killing efficacy than JNJ372 or JNJ-372 analog in NSCLC cell engrafted animal models.

In some embodiments, the antibodies or antigen-binding fragments thereof described herein can block the interaction of cMET and a cMET ligand (e.g., HGF). In some embodiments, the antibodies or antigen-binding fragments thereof decrease binding of cMET to a cMET ligand (e.g., HGF) to less than 90%, less than 80%, less than 70%, less than 60%, less than 50%, less than 40%, less than 30%, less than 20%, less than 10%, or less than 5% as compared to that of an isotype control antibody or JNJ-372 or JNJ-372 analog. In some embodiments, the antibodies or antigen-binding fragments thereof have a cMET/HGF blocking capability (e.g., determined by ELISA) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of JNJ-372 or JNJ-372 analog.

In some embodiments, the antibodies or antigen-binding fragments thereof described herein can block the interaction of EGFR and a EGFR ligand (e.g., EGF). In some embodiments, the antibodies or antigen-binding fragments thereof decrease binding of EGFR to a EGFR ligand (e.g., EGF) to less than 90%, less than 80%, less than 70%, less than 60%, less than 50%, less than 40%, less than 30%, less than 20%, less than 10%, or less than 5% as compared to that of an isotype control antibody or JNJ-372 or JNJ-372 analog. In some embodiments, the antibodies or antigen-binding fragments thereof have a EGFR/EGF blocking capability (e.g., determined by the immunofluorescence assays as described herein) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of JNJ-372 or JNJ-372 analog.

In some embodiments, the antibodies or antigen-binding fragments thereof described herein decrease the phosphorylation (e.g., HGF-induced phosphorylation) level of cMET in cells (e.g., cMET-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody, JNJ372, or JNJ372 analog. In some embodiments, after treatment with the antibodies or antigen-binding fragments thereof, the ratio of cells comprising phosphorylated cMET (e.g., determined by FACS as described herein) is less than or about 50%, less than or about 60%, less than or about 70%, less than or about 80%, less than or about 90%, less than or about 100%, less than or about 110%, less than or about 120%, less than or about 130%, less than or about 140%, less than or about 150%, less than or about 200%, as compared to that after treatment with JNJ-372 or JNJ-372 analog. In some embodiments, after treatment with the antibodies or antigen-binding fragments thereof, the ratio of cells comprising phosphorylated cMET (e.g., determined by FACS as described herein) is less than or about 6%, less than or about 5%, less than or about 4%, less than or about 3%, less than or about 2%, less than or about 1%.

In some embodiments, the antibodies or antigen-binding fragments thereof described herein decrease the phosphorylation (e.g., EGF-induced phosphorylation) level of EGFR in cells (e.g., EGFR-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody, JNJ372, or JNJ-372 analog. In some embodiments, cells (e.g., EGFR-expressing cells) treated with the antibodies or antigen-binding fragments thereof have a ratio of phosphorylated EGFR (e.g., determined by ELISA as described herein) that is less than or about 90%, less than or about 80%, less than or about 70%, less than or about 60%, less than or about 50%, less than or about 40%, less than or about 30%, less than or about 20%, less than or about 10%, less than or about 5%, as compared to that of cells treated with JNJ-372 or JNJ-372 analog.

In some embodiments, the antibodies or antigen-binding fragments thereof described herein decrease the phosphorylation level of downstream signaling pathways involved in cancer cell proliferation, survival, and/or apoptosis. In some embodiments, the antibodies or antigen-binding fragments thereof described herein decrease the phosphorylation level of ERK in cells (e.g., cMET or EGFR-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody, JNJ372, or JNJ372 analog. In some embodiments, cells (e.g., cMET or EGFR-expressing cells) treated with the antibodies or antigen-binding fragments thereof have a ratio of phosphorylated ERK in total ERK (e.g., determined by ELISA as described herein) of less than or about 90%, less than or about 80%, less than or about 70%, less than or about 60%, less than or about 50%, less than or about 40%, less than or about 30%, less than or about 20%, less than or about 10%, less than or about 5%, as compared to that of cells treated with JNJ-372 or JNJ-372 analog. In some embodiments, cells (e.g., cMET or EGFR-expressing cells) treated with the antibodies or antigen-binding fragments thereof have a ratio of phosphorylated ERK over total ERK (e.g., determined by ELISA as described herein) of less than or about 0.95, less than or about 0.90, less than or about 0.85, less than or about 0.80, less than or about 0.75, less than or about 0.75, less than or about 0.70, less than or about 0.65, less than or about 0.60, less than or about 0.55, less than or about 0.50, less than or about 0.45, less than or about 0.40, less than or about 0.35, less than or about 0.30, less than or about 0.25, less than or about 0.20, less than or about 0.15, less than or about 0.10, less than or about 0.05.

In some embodiments, the antibodies or antigen-binding fragments thereof described herein decrease the phosphorylation level of Akt in cells (e.g., cMET or EGFR-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody, JNJ372, or JNJ-372 analog. In some embodiments, cells (e.g., cMET or EGFR-expressing cells) treated with the antibodies or antigen-binding fragments thereof have a ratio of phosphorylated Akt (e.g., determined by ELISA as described herein) of less than or about 90%, less than or about 80%, less than or about 70%, less than or about 60%, less than or about 50%, less than or about 40%, less than or about 30%, less than or about 20%, less than or about 10%, less than or about 5%, as compared to that of cells treated with JNJ-372 or JNJ-372 analog. In some embodiments, cells (e.g., cMET or EGFR-expressing cells) treated with the antibodies or antigen-binding fragments thereof have a ratio of phosphorylated Akt over total Akt (e.g., determined by ELISA as described herein) of less than or about 0.95, less than or about 0.90, less than or about 0.85, less than or about 0.80, less than or about 0.75, less than or about 0.75, less than or about 0.70, less than or about 0.65, less than or about 0.60, less than or about 0.55, less than or about 0.50, less than or about 0.45, less than or about 0.40, less than or about 0.35, less than or about 0.30, less than or about 0.25, less than or about 0.20, less than or about 0.15, less than or about 0.12, less than or about 0.10, less than or about 0.08, less than or about 0.06.

In some embodiments, the antibody (or antigen-binding fragments thereof) specifically binds to an antigen (e.g., a cancer antigen) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, greater than 2×10⁵/Ms, greater than 2.2×10⁵/Ms or greater than 2.4×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁴M, less than 1×10⁻⁵ M, less than 1×10⁻⁶ M, less than 1×10⁻⁷M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, Kd is greater than 1×10⁴M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

In some embodiments, the binding affinity to cMET or EGFR is carefully adjusted, e.g., Kd can be between 100 nM-0.1 nM, between 100 nM-1 nM, between 100 nM-10 nM, between 10 nM-0.1 nM, between 10 nM-1 nM, or between 1 nM-0.1 nM.

General techniques for measuring the affinity of an antibody for an antigen include, e.g., ELISA, RIA, and surface plasmon resonance (SPR).

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the antibodies or antigen binding fragments thereof described herein have the antibody-dependent cell-mediated cytotoxicity (ADCC) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody (e.g., JNJ372 or JNJ-372 analog). In some embodiments, the antibodies or antigen binding fragments thereof described herein can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, 20 folds, or 100 folds, as compared to that of a non-specific antibody control or an isotype antibody control.

In some embodiments, the antibodies or antigen binding fragments thereof described herein have the complement dependent cytotoxicity (CDC) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody (e.g., JNJ372 or JNJ372 analog). In some embodiments, the antibodies or antigen binding fragments thereof described herein can increase complement dependent cytotoxicity (CDC) by at least 10%, 20%, 30%, 40%, 50%, 60%, 70%, 80%, 90%, 100%, 2 folds, 3 folds, 5 folds, 10 folds, 20 folds, or 100 folds, as compared to that of a non-specific antibody control or an isotype antibody control.

In some embodiments, the antibodies or antigen binding fragments thereof described herein have a cell killing IC50 of less than or about 1000 ng/ml, less than or about 500 ng/ml, less than or about 400 ng/ml, less than or about 300 ng/ml, less than or about 250 ng/ml, less than or about 200 ng/ml, less than or about 100 ng/ml. In some embodiments, the antibodies or antigen binding fragments thereof described herein have a cell killing IC50 value that is less than or about 50%, less than or about 60%, less than or about 70%, less than or about 80%, less than or about 90%, less than or about 100%, less than or about 110%, less than or about 120%, less than or about 130%, less than or about 140%, less than or about 150%, less than or about 200% as compared to that of a reference antibody (e.g., JNJ372 or JNJ372 analog). In some embodiments, the antibodies or antigen binding fragments thereof described herein have a cell killing IC50 value that is less than or about 20%, less than or about 10%, less than or about 8%, less than or about 5%, less than or about 3%, less than or about 1% as compared to that of an isotype control antibody.

In some embodiments, the antibodies or antigen binding fragments can increase cMET, EGFR, and/or cMET/EGFR complex internalization rate by at least 10%, 20%, 30%, 40%, 50%, 60%, 70%, 80%, 90%, 100%, 2 folds, 3 folds, 5 folds, 10 folds, or 20 folds as compared to that of a reference antibody (e.g., JNJ372 or JNJ372 analog) or an isotype control antibody.

In some embodiments, the trispecific antibody described herein (e.g., 1H1-cMET/EGFR-Fc(WT)-V3 Tri) can increase the percentage of cells comprising internalized cMET, EGFR, and/or cMET/EGFR complex by at least or about 30%, 40%, 50%, 60%, 70%, 80%, 90%, or 100% as compared to that of a bispecific antibody described herein (e.g., 1H1-cMET/EGFR-Fc(WT)-V3), a reference antibody (e.g., JNJ372 or JNJ372 analog), or an isotype control antibody. In some embodiments, the antibody or antigen-binding fragment thereof described herein can induce cMET, EGFR, and/or cMET/EGFR complex internalization in at least or about 5%, 5.5%, 6%, 6.5%, 7%, 7.5%, 8%, 8.5%, 9%, 9.5%, or 10% cells.

In some embodiments, internalization of the cMET, EGFR, and/or cMET/EGFR complex induced by the antibodies or antigen-binding fragments thereof can further reduce the interaction of cMET with a cMET ligand (e.g., HGF), and/or the interaction of EGFR with an EGFR ligand (e.g., EGF).

In some embodiments, the antibodies or antigen binding fragments can increase phagocytosis rate by at least 10%, 20%, 30%, 40%, 50%, 60%, 70%, 80%, 90%, 100%, 2 folds, 3 folds, 5 folds, 10 folds, or 20 folds as compared to that of a reference antibody (e.g., JNJ372 or JNJ372 analog) or an isotype control.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments can induce apoptosis.

In some embodiments, the antibodies or antigen binding fragments do not have a functional Fc region. For example, the antibodies or antigen binding fragments are Fab, Fab′, F(ab′)2, and Fv fragments.

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., a cMET/EGFR bispecific antibody) or the trispecific antibody described herein (e.g., a cMET/EGFR trispecific antibody) has an asymmetric structure comprising: 2, 3, 4, 5, or 6 antigen binding sites. In some embodiments, the multi-specific antibody described herein comprises 2, 3, 4, 5, or 6 antigen binding sites (e.g., antigen binding Fab domains, scFV, or nanobody (VHH)) that target a cancer antigen (e.g., cMET or EGFR). In some embodiments, the multi-specific antibody described herein (e.g., a cMET/EGFR bispecific antibody, or a cMET/EGFR trispecific antibody) comprises at least 2, 3, 4, 5, 6, or 7 common light chains. In some embodiments, the at least 2, 3, 4, 5, 6, or 7 common light chains have the same VL sequence. In some embodiments, the at least 2, 3, 4, 5, 6, or 7 common light chains have different VL sequences. In some embodiments, the cancer antigen (e.g., cMET or EGFR) binding Fab domain comprises the same VH sequence. In some embodiments, the cancer antigen (e.g., cMET or EGFR) binding Fab domain comprises different VH sequences. In some embodiments, the C-terminus of a cancer antigen binding Fab domain is connected (e.g., covalently connected or chemically connected) to the N-terminus of a neighboring cancer antigen binding Fab domain within the same multi-specific antibody.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides antibodies and antigen-binding fragments thereof that comprise complementary determining regions (CDRs), heavy chain variable regions, light chain variable regions, heavy chains, or light chains described herein. In some embodiments, the antibodies and antigen-binding fragments thereof are imbalanced bispecific antibodies and antigen-binding fragments thereof.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dubel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH. In some embodiments, the linker peptide comprises a sequence that is at least 80%, 85%, 90%, 95%, or 100% identical to any one SEQ ID NOs: 37-42. In some embodiments, the linker peptide comprises a sequence that is at least or about 80%, 85%, 90%, 95%, or 100% identical to SEQ ID NO: 38 or 39. In some embodiments, the linker peptide comprises a sequence that is at least or about 80%, 85%, 90%, 95%, or 100% identical to one or more (e.g., 1, 2, 3, 4, 5, 6, 7, or 8) repeats of GGGGS (SEQ ID NO: 37).

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Linear antibodies comprise a pair of tandem Fd segments (VH-CH1-VH-CH1) which, together with complementary light chain polypeptides, form a pair of antigen binding regions. Linear antibodies can be bispecific or monospecific.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

Antibodies and antibody fragments of the present disclosure can be modified in the Fc region to provide desired effector functions or serum half-life. In some embodiments, the Fc region in any one of the antibody or antigen-binding fragment described herein comprises an aspartic acid (Asp) at position 239 according to EU numbering. In some embodiments, the Fc region in any one of the antibody or antigen-binding fragment described herein comprises a glutamic acid (Glu) at position 332 according to EU numbering. In some embodiments, the Fc region described herein is any one of the Fc regions described herein, comprising an aspartic acid (Asp) at position 239 according to EU numbering, and/or a glutamic acid (Glu) at position 332 according to EU numbering. In some embodiments, the Asp239 and/or Glu332 described herein can increase effector functions (e.g., ADCC or CDC) of an antibody or antigen binding fragment thereof by at least or about 10%, 20%, 30%, 40%, 50%, 60%, 70%, 80%, 90%, 1-fold, 2-fold, 5-fold, 10-fold, 20-fold, 50-fold, or 100-fold, as compared to those of a wild-type antibody or antigen-binding fragment thereof. Details can be found, e.g., in Lazar, G. A. et al., “Engineered antibody Fc variants with enhanced effector function.” Proceedings of the National Academy of Sciences 103.11 (2006): 4005-4010, which is incorporated herein by reference in its entirety.

In some embodiments, the Fc region in any one of the antibody or antigen-binding fragment described herein comprises a wild-type human IgG1 CH2 domain (e.g., SEQ ID NO: 35). In some embodiments, the Fc region in any one of the antibody or antigen-binding fragment described herein comprises a mutated human IgG1 CH2 domain (e.g., SEQ ID NO: 36). In some embodiments, the Fc region described herein comprises a sequence that is at least 80%, 85%, 90%, 95%, or 100% identical to SEQ ID NO: 35 or 36.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multi-specific antibody or antigen-binding fragment thereof described herein (e.g., a cMET/EGFR bispecific antibody or a cMET/EGFR trispecific antibody) binds to an antigen (e.g., cMET) with a binding affinity that is about 50%, about 60%, about 70%, about 80%, about 90%, about 100%, about 110%, about 120%, about 130%, about 140%, about 150%, or about 200% to that of a heavy-chain antibody (e.g., an anti-cMET heavy-chain antibody) comprising the same VHH of the multi-specific antibody.

In some embodiments, the multi-specific antibody or antigen-binding fragment thereof described herein (e.g., a cMET/EGFR bispecific antibody or a cMET/EGFR trispecific antibody) binds to an antigen (e.g., EGFR) with a binding affinity that is about 50%, about 60%, about 70%, about 80%, about 90%, about 100%, about 110%, about 120%, about 130%, about 140%, about 150%, or about 200% to that of an antibody or antigen-binding fragment (e.g., an anti-EGFR monoclonal antibody) comprising the same VH and VL targeting EGFR of the multi-specific antibody.

In some embodiments, the bispecific antibody or antigen-binding fragment thereof described herein (e.g., a cMET/EGFR bispecific antibody, or a cMET/EGFR trispecific antibody) mediates complement-dependent cytotoxicity (CDC) to at least or about 1 fold, 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 30 folds, 40 folds, or 50 folds as compared to that mediated by an isotype control antibody.

In some embodiments, the multi-specific antibody or antigen-binding fragment thereof described herein (e.g., a cMET/EGFR bispecific antibody, or a cMET/EGFR trispecific antibody) is internalized at a percentage that is about 50%, about 60%, about 70%, about 80%, about 90%, about 100%, about 110%, about 120%, about 130%, about 140%, about 150%, or about 200% to that of a monoclonal antibody (e.g., an anti-cMET antibody, an anti-EGFR antibody, or an isotype antibody control).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%. 80%. 85%. 90%. 91%. 92%. 93%. 94%. 95%. 96%. 97%. 98%. 99% identical to any nucleotide sequence as described herein, and an amino acid sequence that is at least 1%, 2%, 3%, 4%, 5%6, 6%, 7%, 8%, 9%, 1%, %15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%7, 7%, 8%, 9%, 10, 1%, 20%, 25%, 30, 35%, 4%0%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies

An isolated fragment of human protein (e.g., cMET, EGFR, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp-->-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wildtype CH3 domain at amino acid position 354 with a bulky hydrophobic amino acid, and/or the second CH3 domain comprises a substitution relative to a wildtype CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q347D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid.

In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Methods of Treatment

The methods described herein include methods for the treatment of disorders associated with cancer. Generally, the methods include administering a therapeutically effective amount of engineered bispecific antibodies (e.g., imbalanced bispecific antibodies) of antigen-binding fragments thereof as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., imbalanced bispecific antibodies) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

In some embodiments, the disclosure features methods that include: identifying a subject having at least one resistance mutation (e.g., C797S, T790M, and/or L858R) in EGFR; and administering a therapeutically effective amount of an antibody or antigen-binding fragment thereof, or an antibody drug conjugate disclosed herein to the subject in need thereof, e.g., a subject having, or identified or diagnosed as having, a cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing cMET. In some embodiments, the cancer is a cancer expressing EGFR. In some embodiments, the cancer is a cancer expressing both cMET and EGFR.

In some embodiments, the cancers are lung cancers (e.g., non-small cell lung cancer).

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is hereditary papillary renal cell carcinomas. In some embodiments, the cancer is gastric, head and neck, liver, ovarian, NSCLC or thyroid cancers.

In some embodiments, the cancer is resistant to the first generation EGFR-TKIs (e.g., gefitinib or erlotinib). In some embodiments, the cancer is resistant to the second generation of EGFR-TKIs (e.g., afatinib or dacomitinib). In some embodiments, the cancer is resistant to the third generation of EGFR-TKIs (e.g., osimertinib (TAGRISSO) or rociletinib).

In some embodiments, the subject (e.g., human) described herein has one or more mutations in EGFR gene in one or more cells. In some embodiments, the mutations include exon 19 deletion mutations and/or a single-point substitution mutation in exon 21 resulting in L858R.

In some embodiments, the subject (e.g., human) described herein has one or more resistance mutations in EGFR protein in one or more cells. In some embodiments, the one or more resistance mutations include C797S, T790M, and/or L858R.

In some embodiments, the cancer cells described herein is cell lines, e.g., NCI-H1975 cells. In some embodiments, the cancer cells comprise EGFR primary and/or secondary activating mutations. In some embodiments, the cancer cells have an elevated cMET level, e.g., at least 10%, at least 20%, at least 30%, at least 40%, at least 50% higher than non-cancerous cells.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, antigen-binding fragment, antibody-drug conjugates, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies, antigen-binding fragments, or antibody-drug conjugates described herein. Two or more (e.g., two, three, or four) of any of the antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Panning ABS VHH Library for cMET Binders

Bispecific antibodies targeting both cMET and EGFR were developed by combining the EGFR binding sequences with cMET binding sequences (e.g., VHH) described herein. The development process can be divided into the following steps: 1) panning a phage VHH library for cMET binders; 2) screening and characterization of lead cMET binder clones; 3) verification of receptor binding and receptor/ligand blocking by cell activation assays; 4) construction and characterization of cMET/EGFR BsAb (bispecific antibody) (e.g., receptor binding, receptor/ligand blocking; receptor phosphorylation, signal transduction repression, and/or cancer killing); 5) optimization and production of lead sequences (e.g., structural optimization and lead characterization); 6) verification of BsAb function using cell-based assays (e.g., receptor binding; receptor/ligand blocking; receptor phosphorylation, signal transduction repression, and/or cancer killing) and/or animal studies (e.g., animal study on the following CDX models: H1975 and H1975-HGF).

Heavy-chain antibody variable domain (VHH) phage library was used to in the panning process. The antigens included human cMET-His-tag protein and transiently-transfected CHO cells expressing human cMET. The panning strategy was designed as follows: 2 rounds of solid-phase phage panning, followed by 1 round of cell-based phage panning. Details of the panning process can be found in the table below.

TABLE 1

| | Round 1 | Round 2 | Round 3 |
|---|---|---|---|
| Antigen | cMET-His-tag | cMET-His-tag | Cells |
| Input | 6 × 10¹¹ | 2.16 × 10¹² | 3.4 × 10¹² |
| Output | 2.3 × 10⁷ | 2.8 × 10⁷ | 6.5 × 10⁵ |
| Enrichment | 2.6 × 10⁴ | 7.7 × 10⁴ | 5.2 × 10⁶ |

After the panning process, ELISA was performed to screen and characterize the lead cMET binders. The primary screening was carried out using two 96-well plates. ELISA readings were recorded. A total of 39 ELISA binders were identified with respective ELISA signal more than 5 folds of the negative control wells. Among these 39 ELISA binders, 16 clones were confirmed to be unique clones.

### Example 2. Secondary Screening-Characterization Through Competitive Ligand Binding

A competitive binding assay (blocking ELISA) was performed to determine cMET binding to human HGF (Hu-HGF) in the presence of the 16 unique clones. A high binding enzyme-linked immunosorbent assay (ELISA) plate was coated with 200 ng/well cMET-FLAG tag protein (50 μl volume; 4 ng/μl) overnight and kept at 4° C. In the next morning, the wells were blocked with 1% bovine serum albumins (BSA) for 2 hours at room temperature (RT). After blocking, the wells were washed four times with PBST (phosphate-buffered saline (PBS) supplemented with 0.05% Tween 20). Next, the antibody samples to be tested (supernatant of the 16 unique clones) or an IgG isotype control were added at a volume of 25 μl to the respective wells and kept at RT for 30 minutes. Then, 25 μl biotinylated Hu-HGF protein was added to the corresponding wells. The plate was kept at RT for 60 minutes. Afterwards, the wells were washed four times with PBST. Streptavidin-HRP conjugated secondary antibody (diluted at 1:1000) was subsequently added and incubated for 30 minutes at RT. The plate was then washed four times with PBST. After washing, the plate was developed by adding the Amplex reagent substrate and then the luminescent signal was measured at 530 nm and 590 nm in a SpectraMax plate reader.

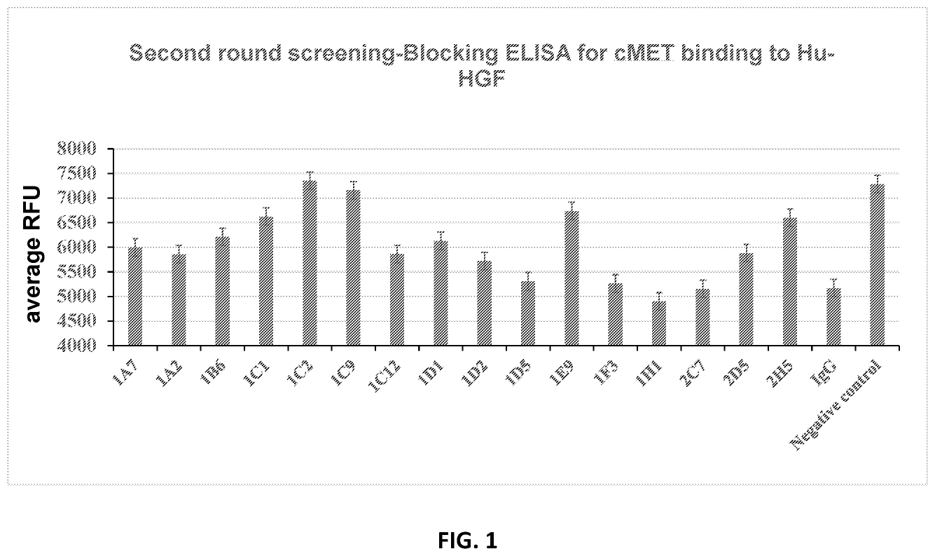

As shown in FIG. 1, four clones 1D5, 1F3, 1H1 and 2C7, were identified to have high competitive ligand binding capability. Thus, they were selected for preparation of VHH mammalian expression systems.

### Example 3. Cross-Reactivity to Cyno-Antigen

To determine the binding similarity between the 16 antibodies to human and monkey (Cynomolgus sp.) antigens, a binding ELISA was performed. A high binding ELISA plate was coated with 1 μg/mL (diluted in 1×PBS) human or monkey antigens and kept at 4° C. overnight. On the next day, the plate was blocked with a blocking buffer (4% non-fat milk in 1×PBS) for 1 hour at 37° C. After blocking, the plate was washed three times with PBST. The antibody samples to be tested were diluted in PBS and then added to the corresponding wells (10 μg/mL). The plate was incubated at 37° C. at RT for 1 hour. After the incubation, the plate was washed three times with the PBST. Next, a secondary antibody (biotinylated anti-c-Myc antibody 9E-10, diluted at 1:500) was added to the wells and the plate was incubated at 37° C. at RT for 1 hour. The plate was then washed three times with PBST. Afterwards, streptavidin-RP, diluted at 1:1000, was added to each well and then the plate was incubated at 37° C. at RT for 1 hour. After the incubation, the plate was washed four times with PBST. After washing, the plate was developed by adding the Amplex reagent substrate and then the luminescent signal was measured at 530 nm and 590 nm in a SpectraMax plate reader.

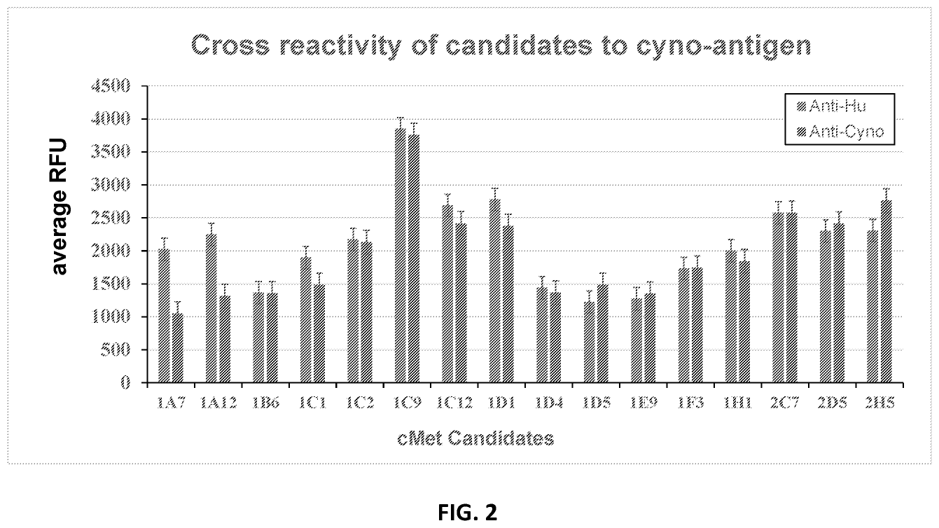

As shown in FIG. 2, 4 clones 1D5, 1F3, 1H1 and 2C7, were identified to have cross reactivity to both human and monkey antigens. Thus, they were selected for preparation of VHH mammalian expression system.

### Example 4. cMET VHH Expression Analysis

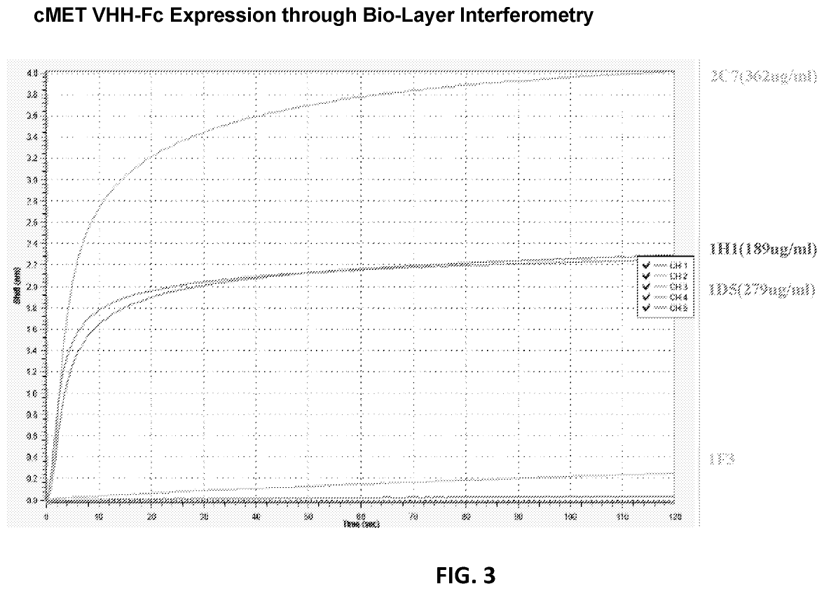

cMET VHH-Fc expression was measured using the Bio-Layer Interferometry (BLI) (Probelife, Palo Alto, CA). The measurement was performed according to the manufacturer's instructions. As shown in FIG. 3, 2C7 was found to have the maximum binding capacity to cMET. However, 2C7 was found to be a mixed clone. 1H1 was identified to be a pure clone. 1D5 was also identified as a mixed clone (i.e., comprising at least two clones).

### Example 5. Testing cMET VHH Binding to cMET by ELISA

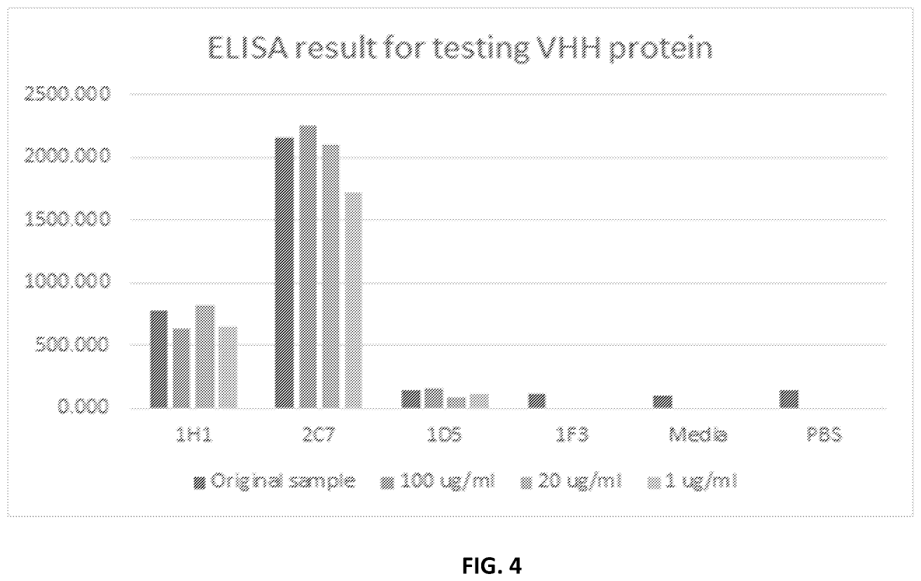

To test the binding of the four identified cMET VHHs to the cMET protein, a binding ELISA was performed. The experiment was carried out substantially the same as described herein. The results are shown in FIG. 4. Because 2C7 was identified as a mixed clone, 1H1 was identified as the clone that exhibited the highest binding capability to cMET.

### Example 6. Competitive ELISA for cMET VHH (Supernatant) Binding to Hu-HGF

To further characterize the cMET VHH clones, a competitive ELISA was performed. A high binding ELISA plate was coated with 4 μg/ml of c-MET-Fc tag protein and kept overnight at 4° C. In the next morning, the wells were blocked with 1% BSA for 2 hours at room temperature (RT). The wells were washed four times with PBST (PBS supplemented with 0.05% Tween 20). Antibody samples to be tested (supernatant of the 1D5, 1H1, and 2C7 clones) or an IgG isotype control (11F11) was added at a volume of 25 μl (with final concentration at 25 μg/ml) to the corresponding wells and kept at room temperature for 30 minutes. Then, 25 μl biotinylated Hu-HGF protein was added (with final concentration at 5 nM) to the corresponding wells and the plate was kept at room temperature for 60 minutes. The wells were washed four times with PBST. Streptavidin-HRP conjugated secondary antibody (diluted at 1:1000) was subsequently added and incubated for 30 minutes at room temperature. The plate was then washed four times with PBST. After washing, the plate was developed by adding the Amplex reagent substrate and then the luminescent signal was measured at 530 nm and 590 nm (cutoff 570 nm) in a SpectraMax plate reader.

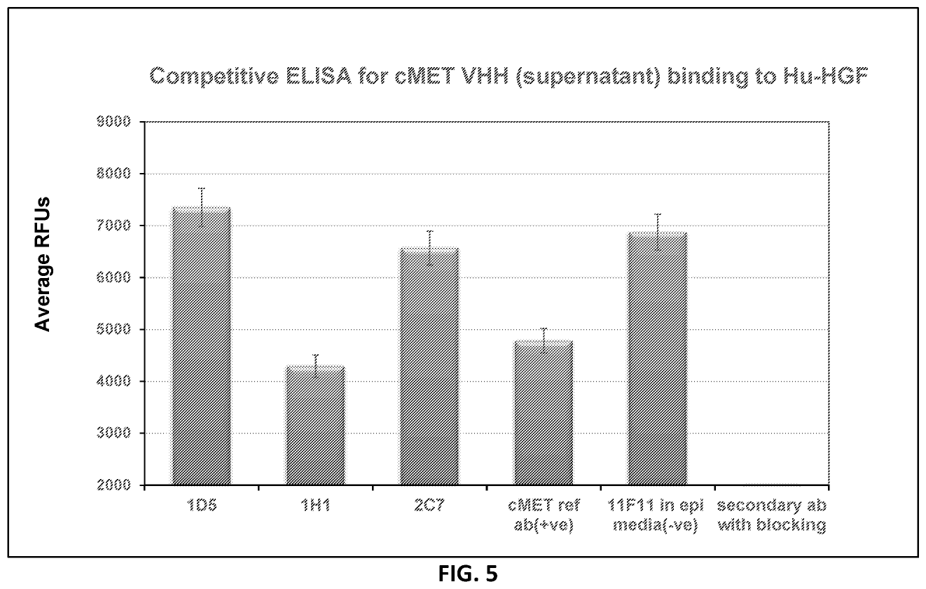

As shown in FIG. 5, 1H1 VHH showed the highest binding capability to cMET within the three clones. In other words, 1H1 VHH exhibited the highest competition to the biotinylated Hu-HGF binding to cMET. Thus, 1H1 VHH was selected for the following experiments.

### Example 7. Construction of the Bi-Specific Ab

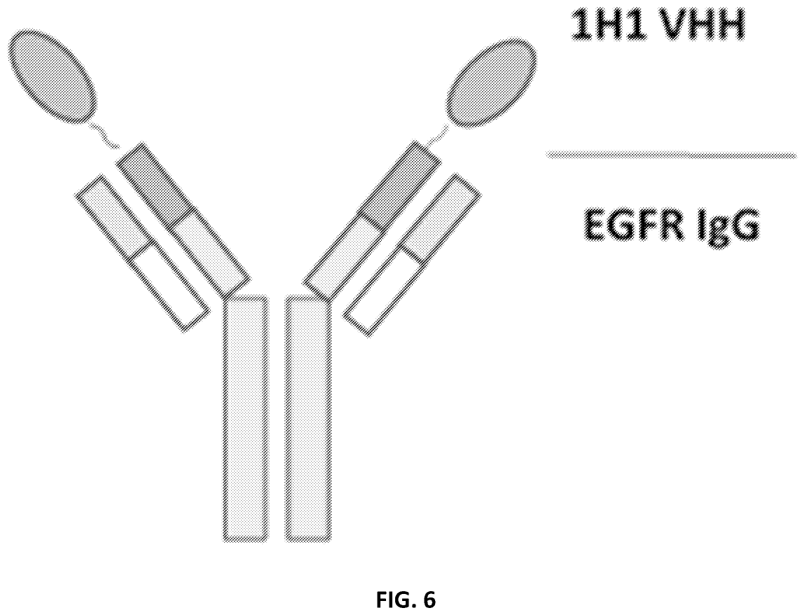

As shown in FIG. 6, a bispecific antibody was prepared using the anti-EGFR IgG as the backbone. An anti-cMET VHH was linked to the heavy chain variable region (VH) via a linker peptide to each arm of the anti-EGFR IgG. The bispecific antibody is named as 1H1-cMET/EGFR-Fc(WT)-V3 when the 1H1 VHH (SEQ ID NO: 19) is linked to both arms of the anti-EGFR IgG (VH: SEQ ID NO: 23; VL: SEQ ID NO: 24) with a wild-type Fc region. Another schematic structure of 1H1-cMET/EGFR-Fc(WT)-V3 is shown in FIG. 18A.

### Example 8. Testing cMET-His Tag Binding with JNJ372 Analog or 1H1-cMET/EGFR-Fc(WT)-V3 BsAb

To evaluate the cMET binding capability of the bispecific antibody 1H1-cMET/EGFR-Fc(WT)-V3 to cMET, a binding ELISA was performed and JNJ372 analog was used as a positive control. Specifically, a high binding ELISA plate was coated with 4 μg/ml of c-MET-His tag protein and kept overnight at 4° C. Binding ELISA was performed using the two BsAbs (with final concentration at 25 μg/ml). In the next morning, the wells were blocked with 1% BSA for 2 hours at room temperature. The wells were washed four times with PBST. The BsAbs or an IgG isotype negative control (11F11) were added to the corresponding wells at a volume of 50 μl and incubated. After the incubation, the wells were washed four times with PBST. Afterwards, HRP-conjugated goat-anti-human Fc antibody was used as the secondary antibody, and added to corresponding wells, followed by an incubation. The plate was then washed four times with PBST. After washing, the plate was developed by adding the Amplex reagent substrate. The luminescent signal was measured at 530 nm and 590 nm (cutoff 570 nm) in a SpectraMax plate reader.

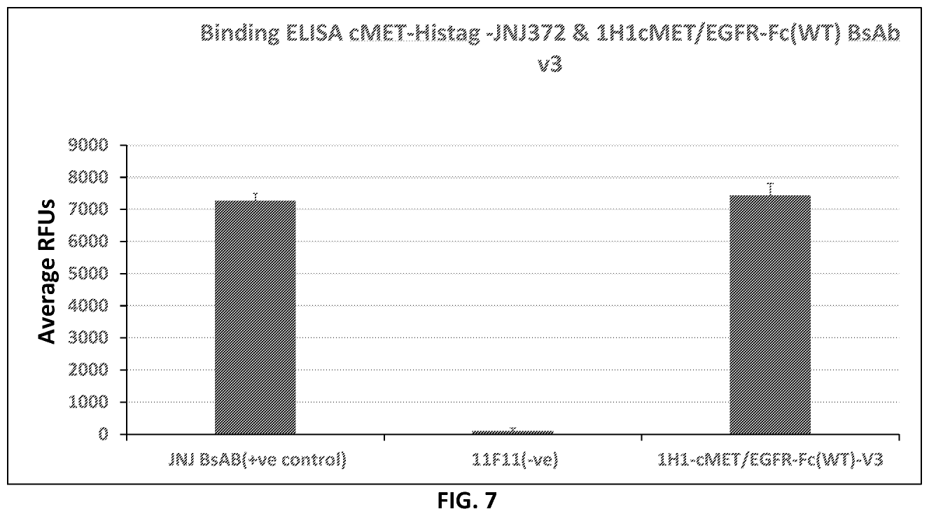

As shown in FIG. 7, 1H1cMET/EGFR-Fc(WT) BsAb showed a high binding capability to the cMET-His tag protein. 1H1cMET/EGFR-Fc(WT) exhibited a similar binding capability to cMET as compared to that of JNJ372 analog.

### Example 9. Testing EGFR-His Tag Binding with JNJ372 Analog or 1H1-cMET/EGFR-Fc(WT)-V3 BsAb

To evaluate the binding capability of the bispecific antibody 1H1-cMET/EGFR-Fc(WT)-V3 to EGFR, a binding ELISA was performed and JNJ372 analog was used as a positive control. Specifically, a high binding ELISA plate was coated with 4 μg/ml of EGFR-His tag protein and kept overnight at 4° C. Binding ELISA was performed using the two BsAbs (with final concentration at 25 μg/ml). In the next morning, the wells were blocked with 1% BSA for 2 hours at room temperature. The wells were washed four times with PBST. The BsAbs or an IgG isotype negative control (11F11) was added to the corresponding wells at a volume of 50 μl and then incubated. After the incubation, the wells were washed four times with PBST. Afterwards, HRP-conjugated goat-anti-human Fc antibody was used as the secondary antibody and added to corresponding well, follow by an incubation. The plate was then washed four times with PBST. After washing, the plate was developed by adding the Amplex reagent substrate. The luminescent signal was measured at 530 nm and 590 nm (cutoff 570 nm) in a SpectraMax plate reader.

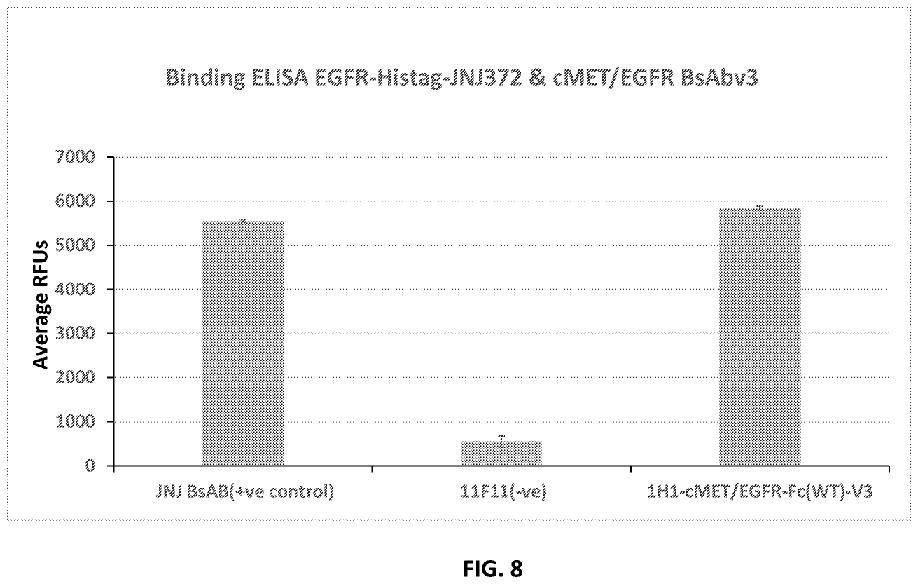

As shown in FIG. 8, 1H1cMET/EGFR-Fc(WT) BsAb showed a high binding capability to the EGFR-His tag protein. 1H1cMET/EGFR-Fc(WT) exhibited a similar binding capability to EGFR as compared to that of JNJ372 analog.

### Example 10. Determination of EC50 for Binding to cMET and EGFR by Titration ELISA

To determine the EC50 of the bispecific antibody 1H1-cMET/EGFR-Fc(WT)-V3, a binding titration ELISA was performed with 5-fold serial dilution of the test antibodies starting from 50 μg/ml to 50,000 ng/ml. JNJ372 analog was used as a positive control. The dilution concentrations were 50 μg/ml, 10 μg/ml, 2 μg/ml, 0.4 μg/ml, 0.08 μg/ml and 0.016 μg/ml.

Specifically, high binding ELISA plates were coated with 4 μg/ml of c-MET-His tag or EGFR-His tag protein and kept overnight at 4° C. In the next morning, the wells were blocked with 1% BSA for 2 hours at room temperature. After blocking, the wells were washed four times with PBST. The diluted antibody samples were added to the corresponding wells at a volume of 50 μl and incubated. After the incubation, the wells were washed four times with PBST. Afterwards, HRP-conjugated goat-anti-human Fc antibody was used as the secondary antibody and added to corresponding wells, followed by an incubation. The plate was then washed four times with PBST. After washing, the plate was developed by adding the Amplex reagent substrate. The luminescent signal was measured at 530 nm and 590 nm (cutoff 570 nm) in a SpectraMax plate reader.

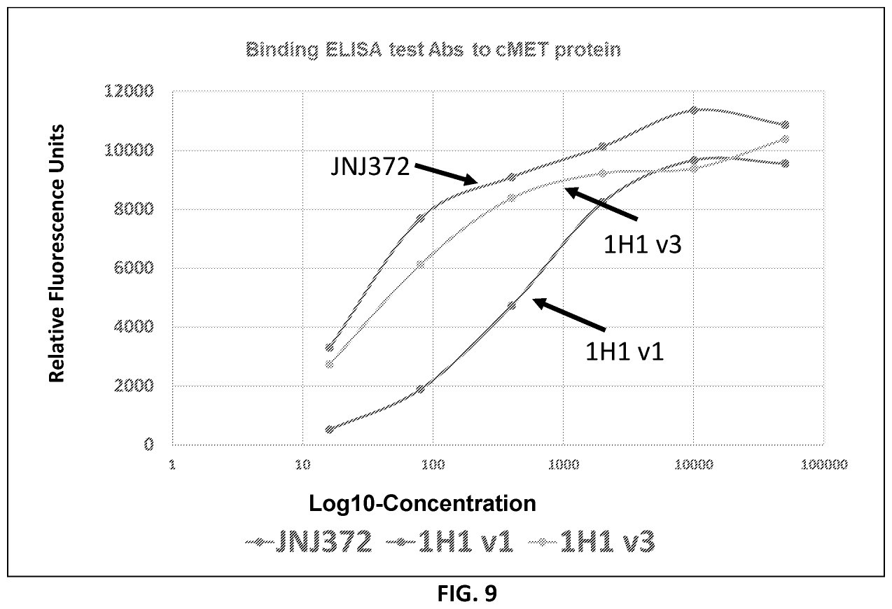

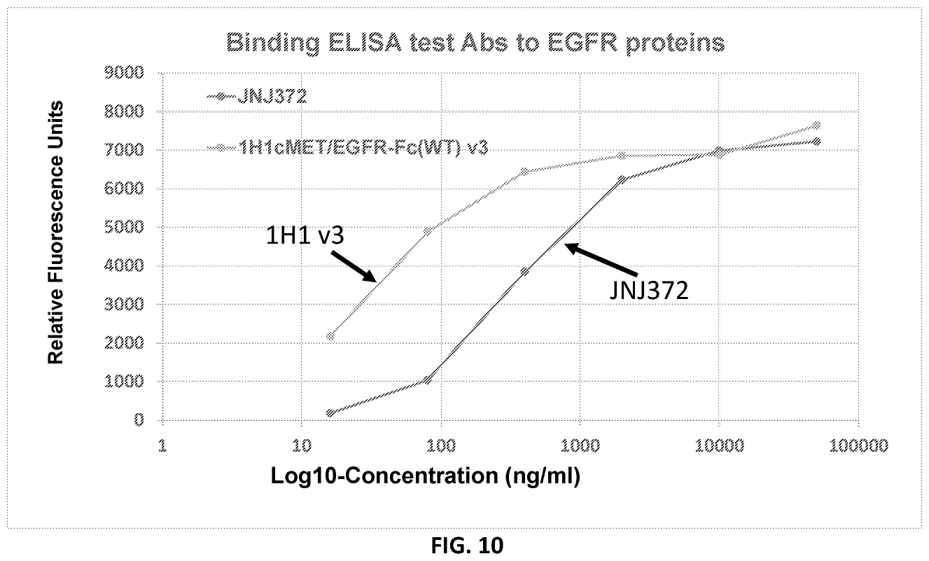

As shown in FIG. 9, the EC50 values for cMET binding was determined as 8.6077 ng/ml and 4.8871 ng/ml for 1H1-cMET/EGFR-Fc(WT)-V3 and JNJ372 analog, respectively. The schematic structure of 1H1-cMET/EGFR-Fc (WT)-V1 is shown in FIG. 16A. As shown in FIG. 10, the EC50 values for EGFR binding was determined as 9.27 ng/ml and 355.95 ng/ml for 1H1-cMET/EGFR-Fc(WT)-V3 and JNJ372 analog, respectively.

### Example 11. Determination of K_{D}, Kₒₙ, and K_{off} for cMET Binding to JNJ372 Analog and 1H1cMET/EGFR(WT)

The K_{D}, Kₒₙ and K_{off} values binding to cMET were calculated using the Bio-Layer Interferometry (BLI) technology (Probelife, Palo Alto CA) according to the manufacturer's instructions. The results are shown in the tables below.

TABLE 2
cMET/1H1-cMET/EGFR-Fc (WT)-V3

| BsAb Conc. (nM) | koff(1/s) | kon(1/Ms) | KD(M) |
|---|---|---|---|
| 114 | 7.85 × 10⁻⁴ | 2.48 × 10⁶ | 3.17 × 10⁻¹⁰ |
| 38.1 | 7.85 × 10⁻⁴ | 2.48 × 10⁶ | 3.17 × 10⁻¹⁰ |
| 12.7 | 7.85 × 10⁻⁴ | 2.48 × 10⁶ | 3.17 × 10⁻¹⁰ |
| 4.23 | 7.85 × 10⁻⁴ | 2.48 × 10⁶ | 3.17 × 10⁻¹⁰ |
| 1.41 | 7.85 × 10⁻⁴ | 2.48 × 10⁶ | 3.17 × 10⁻¹⁰ |

TABLE 3
cMET/JNJ372 analog

| BsAb Conc. (nM) | koff(1/s) | kon(1/Ms) | KD(M) |
|---|---|---|---|
| 133 | 9.13 × 10⁻⁴ | 2.25 × 10⁶ | 4.05 × 10⁻¹⁰ |
| 44.4 | 9.13 × 10⁻⁴ | 2.25 × 10⁶ | 4.05 × 10⁻¹⁰ |
| 14.8 | 9.13 × 10⁻⁴ | 2.25 × 10⁶ | 4.05 × 10⁻¹⁰ |
| 4.94 | 9.13 × 10⁻⁴ | 2.25 × 10⁶ | 4.05 × 10⁻¹⁰ |
| 1.65 | 9.13 × 10⁻⁴ | 2.25 × 10⁶ | 4.05 × 10⁻¹⁰ |

In a separate experiment, the K_{D} values binding to cMET or EGFR were determined using JNJ372 analog, 1H1-cMET/EGFR-Fc (WT)-V2, or 1H1-cMET/EGFR-Fc (WT)-V3. The schematic structure of 1H1-cMET/EGFR-Fc (WT)-V2 and 1H1-cMET/EGFR-Fc (WT)-V3 are shown in FIG. 17A and FIG. 18A, respectively. The results are shown in the table below.

TABLE 4

| | K_{D} (M) | K_{D} (M) |
|---|---|---|
| BsAb | (cMET binding) | (EGFR binding) |
| JNJ372 analog | 4.32 × 10⁻¹⁰ | 6.89 × 10⁻¹⁰ |
| 1H1-cMET/EGFR-Fc (WT)-V2 | 1.88 × 10⁻¹⁰ | 3.25 × 10⁻¹⁰ |
| 1H1-cMET/EGFR-Fc (WT)-V3 | 2.39 × 10⁻¹⁰ | 2.57 × 10⁻¹⁰ |

### Example 12. Testing cMET/HGF Blocking Using JNJ372 Analog and 1H1-cMET/EGFR-Fc(WT)-V3 by Competitive ELISA

To evaluate the effects of JNJ372 analog and 1H1-cMET/EGFR-Fc(WT)-V3 blocking the interaction of cMET and its ligand HGF, a competitive ELISA was performed. A high binding ELISA plate was coated with 4 μg/ml of c-MET-His tag protein and kept overnight at 4° C. Competitive ELISA was performed using the antibody samples (with final concentration at 25 μg/ml) and an isotype control (11F11) as described in Example 6. The effects of the antibody samples competing with biotinylated human HGF were determined. Streptavidin-HRP conjugated secondary antibody was used.

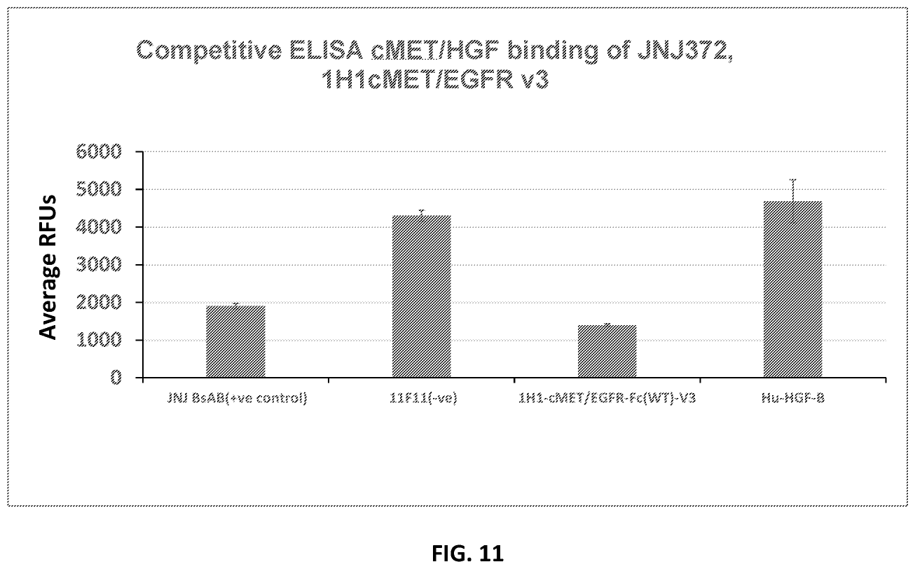

As shown in FIG. 11, 1H1-cMET/EGFR-Fc (WT)-V3 was more effective in blocking cMET/HGF interaction as compared to that of JNJ372 analog.

### Example 13. Determination of EGFR/EGF Blocking Using JNJ372 Analog and 1111-cMET/EGFR-Fc(WT)-V3 by Competitive Binding Assays

To determine whether 1H1-cMET/EGFR-Fc(WT)-V3 can block the interaction between EGF and EGFR, microscopy and flow cytometry assays were used. NCIH1975 cells (ATCC® CRL-5908) were plated overnight in a 48 well plate (2×10⁵ cells per/well) in the RPMI1640 media supplemented with 10% FBS. In the next morning, the cells were washed three times with 1×PBS. Afterwards, the cells were fixed with 4% paraformaldehyde in PBS for 15 minutes at RT. The cells were then washed three times with 1×PBS, followed by blocking in 10% Normal Goat Serum (NGS) for 30 minutes at RT. After blocking, the NGS was removed. JNJ372 analog, 1H1-cMET/EGFR-Fc(WT)-V3, and an isotype control (11F11) were added to corresponding wells, dissolved in NGS, at a concentration of 20 μg/ml and kept at RT for 2 hours. Afterwards, biotinylated EGF was added at a final concentration of 2 μg/ml, and the plate was kept at RT for one hour.

Next, the cells were washed five times with 1×PBS. A secondary antibody (streptavidin PE conjugated) was dissolved in NGS at a final dilution ratio of 1:50, and was then added to the wells, followed by an incubation for 1 hour at RT. Subsequently, the cells were washed six times with 1×PBS and imaged under a fluorescence microscope. After the imaging step, the cells were washed three times in a FACS (fluorescence-activated cell sorting) buffer, and flow cytometry was performed to quantify the EGF/EGFR blocking effects of the tested antibodies.

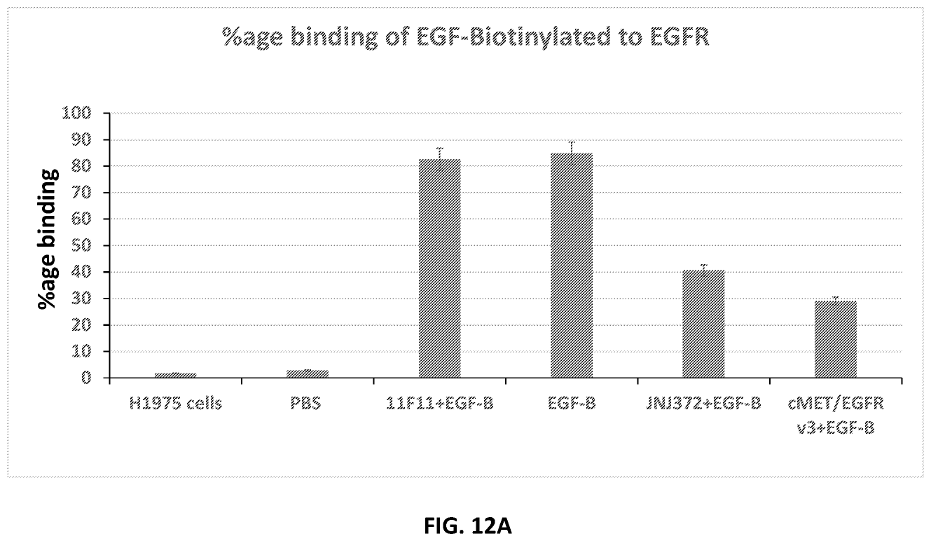

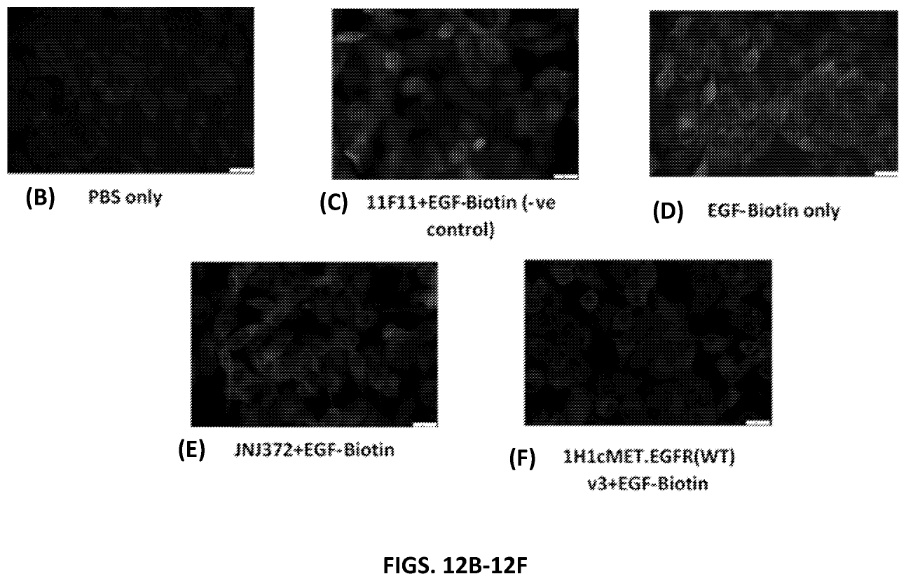

As shown in FIGS. 12A-12F, 1H1-cMET/EGFR-Fc(WT)-V3 was more effective than JNJ372 analog in blocking the interaction between EGF and EGFR.

### Example 14. Determination of Receptor Phosphorylation of cMET and EGFR in NCI-H1975 Cells Treated with JNJ372 Analog or 1H1-cMET/EGFR-Fc(WT)-V3

NCI-H1975 cells are human cancer cell lines with EGFR primary and secondary activating mutations, which confer resistance to EGFR tyrosine kinase inhibitors (TKIs), in addition to MET gene amplification. In these cell lines the ligands of EGFR and cMET, EGF and HGF respectively, can induce phosphorylation of the EGFR and cMET receptors, which stimulates downstream signaling through the phosphorylation of ERK and Akt proteins. To determine whether 1H1-cMET/EGFR-Fc(WT)-V3 can inhibit the ligand-induced phosphorylation of EGFR and cMET, NCI-H1975 cells were cultured in the presence of EGF and HGF respectively, with JNJ372 analog or 1H1-cMET/EGFR-Fc(WT)-V3.

Phospho-cMET was detected using flow cytometry assays. Specifically, NCI-H1975 cells were grown overnight in cell starvation media (RPMI media supplemented with glutamax (diluted from the 100×solution)), with 50000 cells/well in a 24-well plate. In the next morning, the media was discarded and the cells were treated with the test antibodies (1.5 μg/ml), dissolved in 250 μl/well of the starvation media, for 1 hour at 37° C. and 5% CO₂. Subsequently, cells were treated with 200 ng/ml of HGF, dissolved in 250 μl/well of the starvation media, for 15 minutes at 37° C. and 5% CO₂. Final HGF concentration was 100 ng/ml. After the 15-minute treatment, the media was discarded and cells were washed once with ice-cold 1×PBS. The cells were then fixed with 4% paraformaldehyde (PFA) for 15 minutes at RT. After fixation, the cells were washed three times with 1×PBS and stored at 4° C. overnight in PBS. In the next morning, cells were permeabilized with 0.1% Triton X-100 in PBS for 4-5 minutes on ice, washed three times with PBS, and then blocked in 10% NGS (normal goat serum) for 30 minutes. After blocking, the primary antibody (Human/Mouse Phospho-HGFR/c-MET (Y1349) (AF3950); Novusbio, Biotechne, MN)) was added to the corresponding wells (15 μg/ml dissolved in 10% NGS) for 2 hours at RT. Subsequently, the cells were washed three times with PBS, and then the secondary antibody (Goat anti-Rabbit IgG (H+L) DyLight 594; Invitrogen, Thermo Fisher, MA) diluted at a 1:25 ratio in FACS buffer (2% FBS in PBS) was added to the cells. The cells were then washed three times with PBS and then the scraped from the wells in FACS buffer. Afterwards, the cell were washed three times with FACS buffer and then analyzed by the flow cytometer (BD FACS Calibur, NJ).

Phospho-EGFR was detected using an ELISA kit, PathScan® Phospho-EGF Receptor (Tyr1173) Sandwich ELISA Kit #7187 (Cell Signaling Tech). Similar to detecting the phosphor-cMET as described herein, NCI-H1975 cells were grown overnight in cell starvation media (RPMI media supplemented with glutamax (diluted from the 100×solution)), with 50000 cells/well in a 24-well plate. In the next morning, the media was discarded and the cells were treated with the test antibodies (1.5 μg/ml), dissolved in 250 μl/well of starvation media, for 1 hour at 37° C. and 5% CO₂. Subsequently, cells were treated with 100 ng/ml of EGF, dissolved in 250 μl/well of starvation media, for 15 minutes at 37° C. and 5% CO₂. Final EGF concentration was 50 ng/ml. After the 15-minute treatment, the media was discarded and cells were washed once with ice-cold 1×PBS. Then, 300 ul of 1×lysis buffer supplemented with 1 mM phenylmethylsulfonyl fluoride (PMSF) was added to the wells and the plate was kept at 4° C. for 5 minutes. The cells were scraped off the plate and then collected in 1.5 ml tubes. The cell lysates were passed through a small-diameter needle several times to break the cells. Afterwards, the cell lysates were centrifuged at 13200 rpm for 10 minutes at room temperature. The supernatant collected after the centrifugation was used as the cell lysate for the ELISA. The ELISA for detecting phosphor-EGFR was performed according to the manufacturer's instructions.

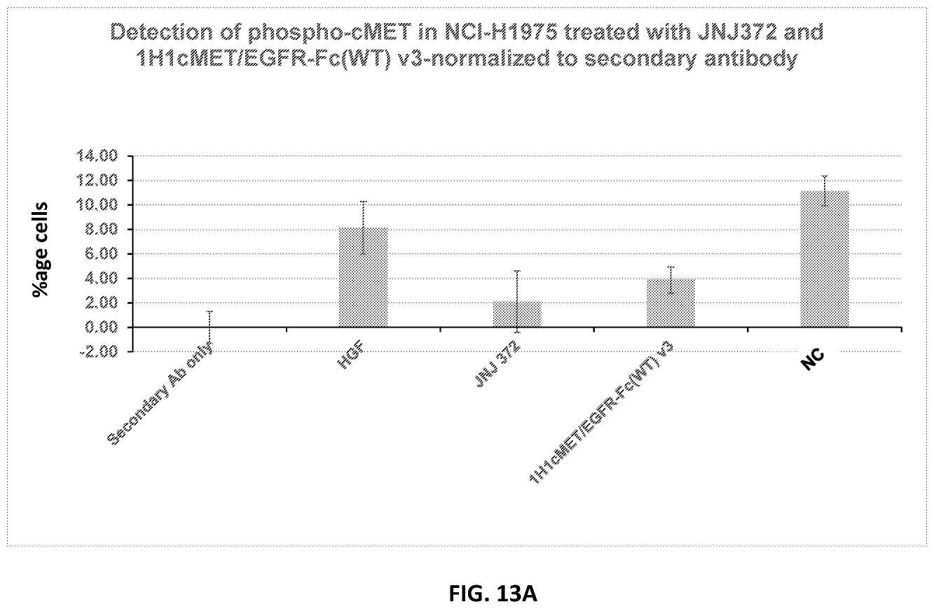

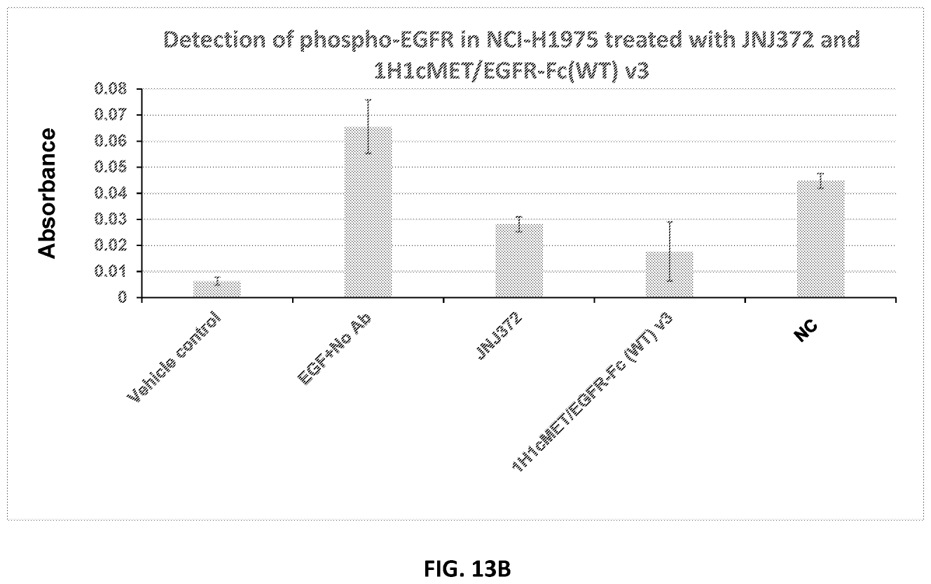

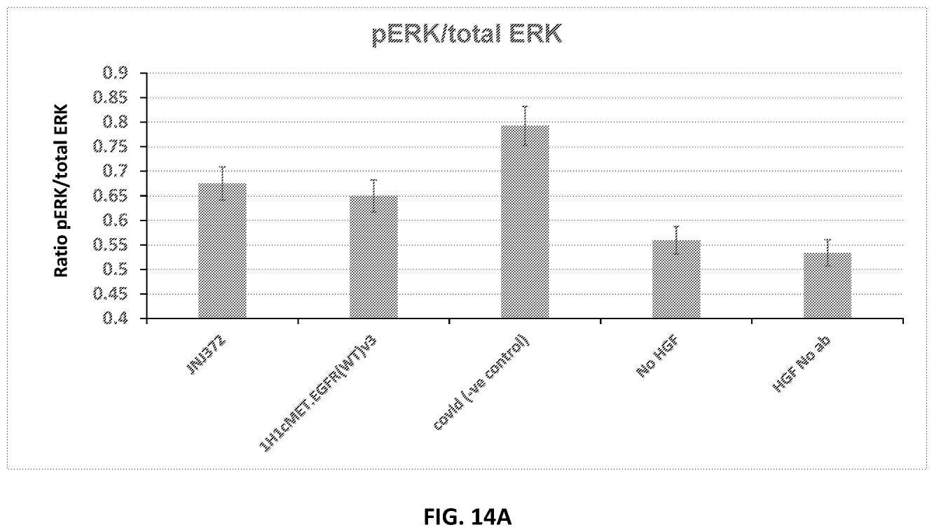

As shown in FIGS. 13A-13B, 1H1-cMET/EGFR-Fc(WT)-V3 was found to be more effective than JNJ372 analog in decreasing the phosphorylation of EGFR (FIG. 13B), whereas JNJ372 analog decreased the cMET phosphorylation more than that of 1H1-cMET/EGFR-Fc(WT)-V3 (FIG. 13A).

### Example 15. Detection of pERK and pAkt in JNJ372 Analog or 1H1-cMET/EGFR-Fc(WT)-V3-Treated NCI-111975 Cells

Functional assays were performed to further test the bispecific antibody 1H1-cMET/EGFR-Fc(WT)-V3. Specifically, the phosphorylation states of ERK and Akt proteins were determined. ERK and Akt proteins are involved in downstream signaling pertaining to cancer cell proliferation, survival and obstructing the apoptotic pathways. NCIH1975 cells (ATCC® CRL-5908) were grown overnight in a 24-well plate (2×10⁵ cells/well) in RPMI1640 media supplemented with 10% FBS and 8 ng/ml Hu-HGF. To detect pERK, ERK1/2 (pT202/Y204+Total) ELISA Kit (ab176660) from Abcam (Cambridge, UK) was used. To detect pAkt, PathScan© Phospho-Akt1 (Ser473) Sandwich ELISA Kit #7160 from Cell Signalling Technology (Danvers, MA, US) was used. The ELISAs were performed according to the instructions of the respective manufacturers. Protein concentrations were determined using the Pierce™ Coomassie Plus (Bradford) Assay Kit (150 μl of the reagent (with 5 μl of lysate) per well in a 96-well plate).

For phosphor-ERK (pERK) detection, cells were treated with the bispecific antibodies (1.5 μg/ml) for 30 minutes. Subsequently, the media was discarded, and the cells were rinsed twice with ice-cold PBS. Then, 350 μl of 1×lysis buffer (supplemented with 1 mM PMSF as the protease inhibitor) was added to the wells and the lysed cells were collected in 1.5 ml tubes. The cell lysate was used according to the ELISA protocol.

Similarly for phosphor-Akt (pAkt) detection, cells were treated with the bispecific antibodies (1.5 μg/ml) for 60 minutes. The media was then discarded and the cells were rinsed twice with ice-cold PBS. Then, 350 μl of 1×lysis buffer (supplemented with 1 mM PMSF as the protease inhibitor) was added to the wells and the plate was kept on ice for 5 minutes. Afterwards, the cells were scraped and collected in 1.5 ml tubes. Then, the lysed cells were passed through a small-diameter needle several times to break the cells. Subsequently, the cell lysate was centrifuged at 13200 rpm for 10 minutes at room temperature. The supernatant collected after the centrifugation was used as the cell lysate to perform the pAkt ELISA.

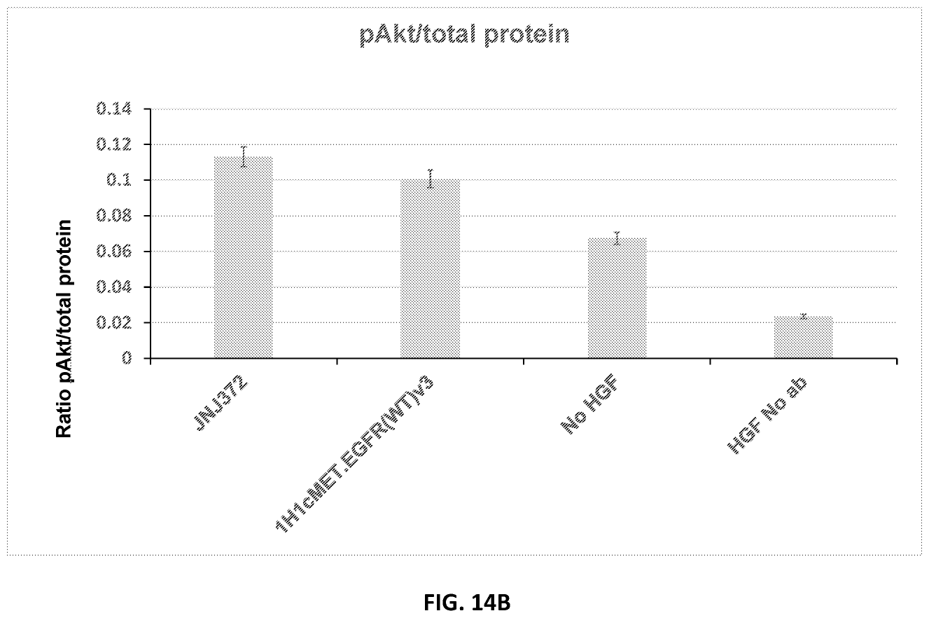

As shown in FIGS. 14A-14B, 1H1-cMET/EGFR-Fc(WT)-V3 was found to be more effective than JNJ372 analog in decreasing the ratio of pERK (over total ERK) and the ratio of pAkt (over total Akt) in NCI-H1975 cells grown in the presence of Hu-HGF.

### Example 16. Determination of Effector Functions of 1H1-cMET/EGFR-Fc(WT)-V3 in Comparison to JNJ372 Analog

Increasingly high levels of EGFR and cMET on the surface of tumor cells allows for specific targeting and killing of these cells, by immune effector cells (e.g., NK cells) through Fc-dependent effector mechanisms, e.g., antibody-dependent cellular cytotoxicity (ADCC) and complement dependent cytotoxicity (CDC). To determine the effector functions of 1H1-cMET/EGFR-Fc(WT)-V3 in comparison to JNJ372 analog, ADCC and CDC assays were performed as described below.

Before start of both assays, NCI-H1975 cells were labelled with Calcein AM cell-permeant dye. Afterwards, the cells were briefly washed with PBS, and then treated with 0.5 M ethylenediaminetetraacetic acid (EDTA) for 20 minutes. Subsequently, the treated cells were centrifuged at 1200 rpm for 5 minutes, counted and then resuspended in 4 ml of serum free media (˜3.7 million cells were used for labelling). Then, 8 μl of 1 mM Calcein AM (as stock solution) to was added to the cells (at a final concentration of 2 μM calcein). The cells were then incubated for 30 minutes in dark at RT. Afterwards, the cells were centrifuged again at 1200 rpm for 5 minutes to remove the media. After media removal, the cells were washed twice with serum-free media, and then aliquoted equally in two separate 15 ml tubes in 4.6 ml media. The media was directly used for the CDC determination assay below, and supplemented with 10% FBS for the ADCC determination assay below.

### Antibody-Dependent Cell-Mediated Cytotoxicity (ADCC)

Calcein labelled cells were added to a U-bottom 96-well plate at 100 μl per well. The test antibodies were added at a dilution of 80 μg/ml in 50 μl media with a final concentration of 20 μg/ml. Then, 360,000 NK92-CD16 cells/well in 50 μl media were added to the labelled H1975 cells. The ratio of the Effector cells to target cells was 9:1. The cell mixture was incubated at 37° C. for 2 hours and 45 minutes. Then 30 μl of 10× lysis buffer was added to maximum release wells and 30 μl of RPMI media was added to maximum release control wells. The plate was further incubated for 15 minutes. After the incubation, the plate was centrifuged at 1200 rpm for 5 minutes, and 50 μl of the obtained supernatant was transferred to a clear well black flat-bottom 96-well plate (Greiner). Then, 50 μl PBS was added to the supernatant. Afterwards, the luminescent signal of the plate was measured at 485 nm and 538 nm (cutoff 530 nm) in a Spectramax Fluorescence plate reader.

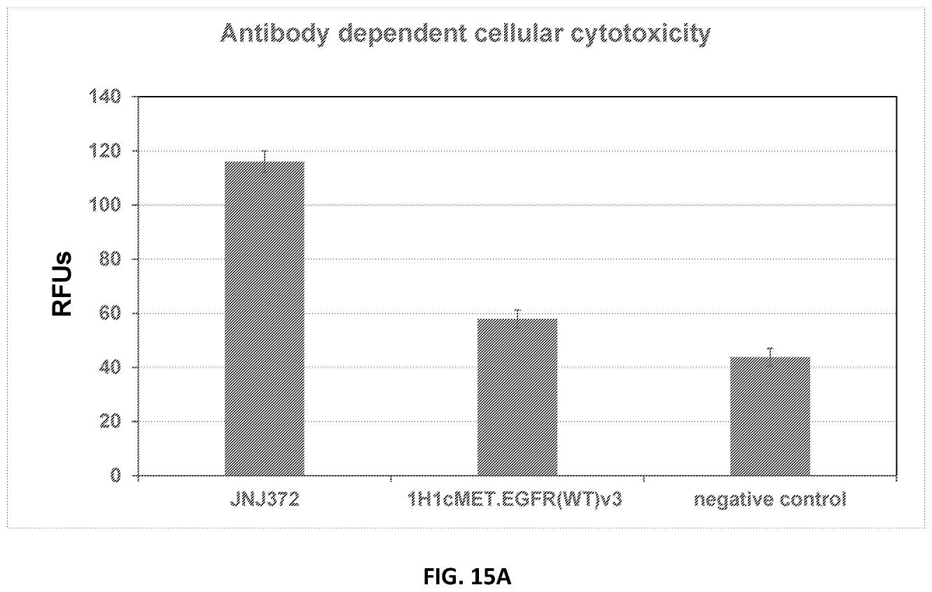

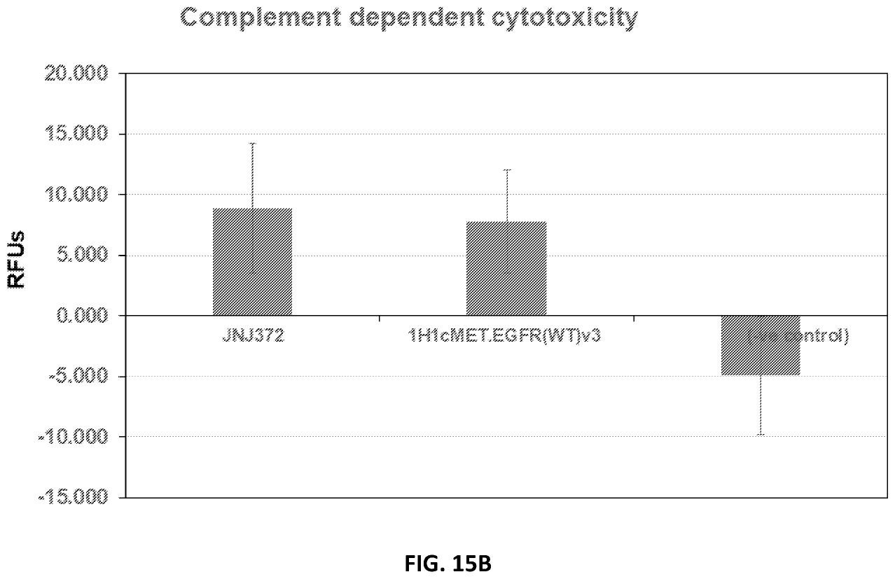

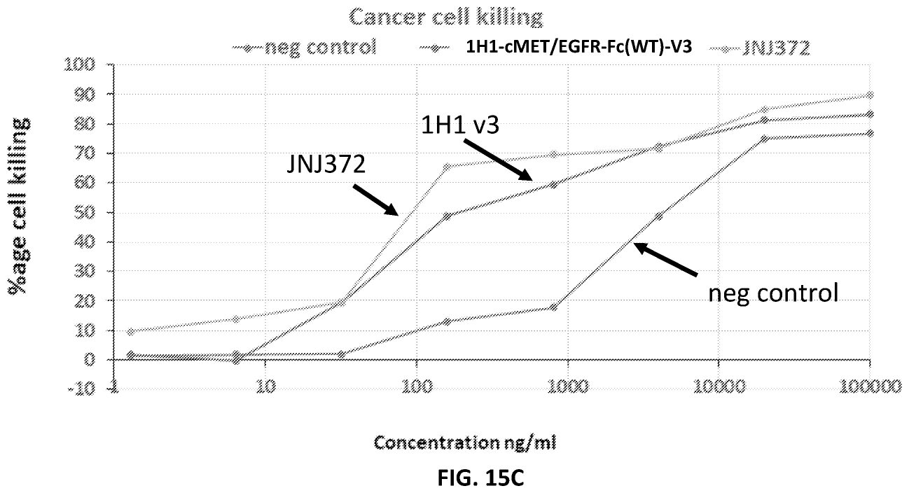

As shown in FIG. 15A, JNJ372 analog was more effective in ADCC-induced cell killing as compared to 1H1-cMET/EGFR-Fc(WT)-V3. It is possible that JNJ372 analog has an built-in mutation in the Fc region for ADCC functions whereas 1H1-cMET/EGFR-Fc(WT)-V3 has a wild type Fc region. Thus, 1H1-cMET/EGFR-Fc(WT)-V3 can be mutated in the Fc region to increase its ADCC effect.

### Complement-Dependent Cytotoxicity (CDC)

Calcein labelled cells were added to a U-bottom 96-well plate at 100 μl per well. The test antibodies were added at a dilution of 80 μg/ml in 50 μl media with a final concentration of 20 μg/ml. Complement serum was added at 20% dilution in 50 μl media at a final concentration of 5%. The cells were incubated at 37° C. for 2 hours and 45 minutes. Then, 30 μl of 10× lysis buffer was added to maximum release wells and 30 μl of RPMI media was added to maximum release control wells. The plate was further incubated for 15 minutes. After the incubation, the plate was centrifuged at 1200 rpm for 5 minutes and 50 μl of the obtained supernatant was transferred to a clear well black flat-bottom 96-well plate (Greiner). Then, 50 μl PBS was added to the supernatant. Afterwards, the luminescent signal of the plate was measured at 485 nm and 538 nm (cutoff 530 nm) in a Spectramax Fluorescence plate reader.

As shown in FIG. 15B, 1H1-cMET/EGFR-Fc(WT)-V3 was found to be as effective in CDC as compared to JNJ372 analog.

The cancer cell killing capability of 1H1-cMET/EGFR-Fc(WT)-V3 and JNJ372 analog at different concentrations were also evaluated. The IC50 results are shown in the table below.

TABLE 5

| Antibody | Cell killing IC50 (ng/ml) |
|---|---|
| JNJ372 analog | 195.2186 |
| 1H1-cMET/EGFR-Fc(WT)-V3 | 214.6154 |
| Negative Control (isotype) | 2594.843 |

As shown in FIG. 15C, 1H1-cMET/EGFR-Fc(WT)-V3 exhibited similar cell killing capability as compared to that of JNJ372 analog.

### Example 17. Conclusions

According to the experimental results as described herein, the follow table provides a summary of the receptor binding, receptor/ligand blocking, signal transduction, and cancer cell killing effects of the bispecific antibodies described herein.

TABLE 6

| | Receptor | Receptor/Ligand | | Cancel cell |
|---|---|---|---|---|
| BsAb | binding | blocking | Signal Transduction | killing |
| 1H1- | cMET | cMet/HGF | cMET receptor | ++ |
| cMET/EGFR- | binding | ++ | phosphorylation | |
| Fc(WT)-V1 | ++ | EGFR/EGF | ++ | |
| | EGFR | ++ | EGFR receptor | |
| | binding | | phosphorylation | |
| | ++ | | +++ | |
| | | | pERK/totalERK | |
| | | | + | |
| | | | pAkt/total protein | |
| | | | + | |
| 1H1- | cMET | cMET/HGF | cMET receptor | N/A |
| cMET/EGFR- | binding | +++ | phosphorylation | |
| Fc(WT)-V2 | +++ | EGFR/EGF | N/A | |
| | EGFR | ++ | EGFR receptor | |
| | binding | | phosphorylation | |
| | +++ | | N/A | |
| | | | pERK/totalERK | |
| | | | ++ | |
| | | | pAkt/total protein | |
| | | | + | |
| 1H1- | cMET | cMET/HGF | cMET receptor | +++ |
| cMET/EGFR- | binding | +++ | phosphorylation | |
| Fc(WT)-V3 | +++ | EGFR/EGF | +++ | |
| | EGFR | ++++ | EGFR receptor | |
| | binding | | phosphorylation | |
| | +++ | | +++ | |
| | | | pERK/totalERK | |
| | | | ++ | |
| | | | pAkt/total protein | |
| | | | +++ | |
| JNJ372 | cMET | cMET/HGF | cMET receptor | +++ |
| analog | binding | ++ | phosphorylation | |
| | +++ | EGFR/EGF | ++ | |
| | EGFR | +++ | EGFR receptor | |
| | binding | | phosphorylation | |
| | +++ | | ++ | |
| | | | pERK/totalERK | |
| | | | +++ | |
| | | | pAkt/total protein | |
| | | | ++ | |

The schematic structure of 1H1-cMET/EGFR-Fc (WT)-V1, 1H1-cMET/EGFR-Fc (WT)-V2, and 1H1-cMET/EGFR-Fc (WT)-V3 are shown in FIG. 16A, FIG. 17A, and FIG. 18A, respectively.

### Example 18. Internalization Induced by EGFR/cMET Multispecific Antibodies

Experiments were performed to assess internalization induced by EGFR/cMET bispecific and trispecific antibodies. Specifically, internalization assays were performed using JNJ372 analog, the EGFR/cMET bispecific antibody 1H1-cMET/EGFR-Fc(WT)-V3, and an EGFR/cMET trispecific antibody 1H1cMET/1A12cMET/EGFR(WT) (with the TriSpecific-V3 structure as shown in FIG. 18B). MDA231 cells were mixed with 20 μg/ml antibodies, and incubated at 37° C. for 30 minutes. Then pHrodo-labeled secondary antibody was added and incubated with the cells at 37° C. for 24 hours. Cells were then harvested and analyzed by FACS.

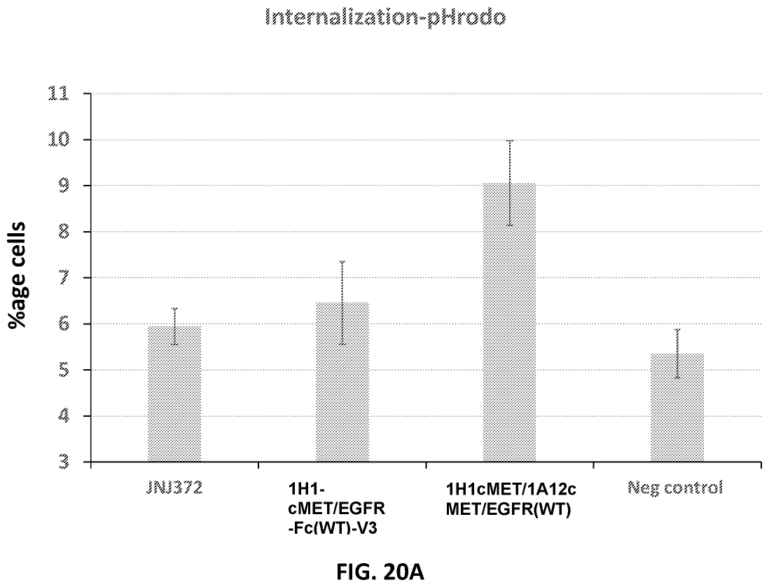

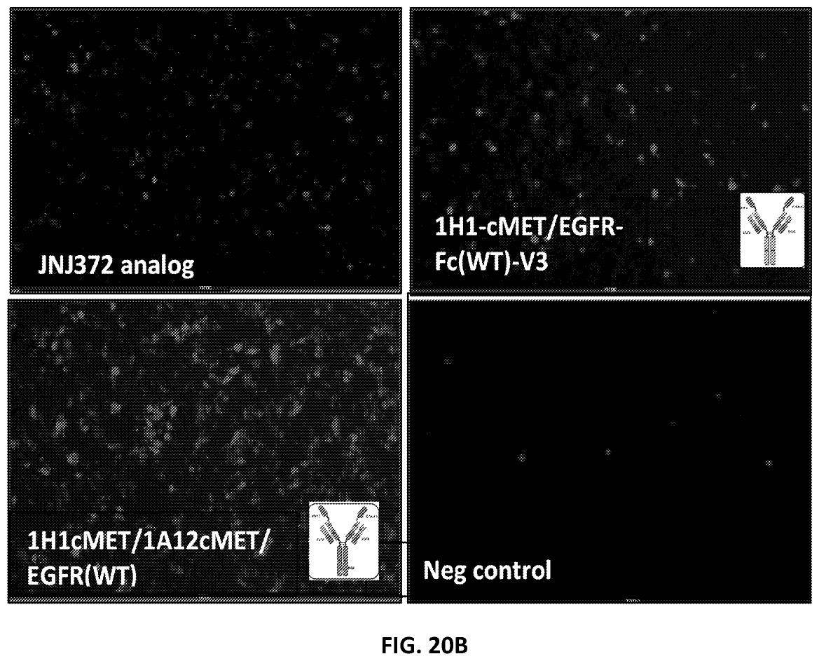

As shown in FIGS. 20A-20B, 1H1cMET/1A12cMET/EGFR(WT) showed enhanced internalization of the cMET/EGFR complex as compared to JNJ372 analog or 1H1-cMET/EGFR-Fc (WT)-V3.

### Example 19. cMET VHH Epitope Binning Analysis

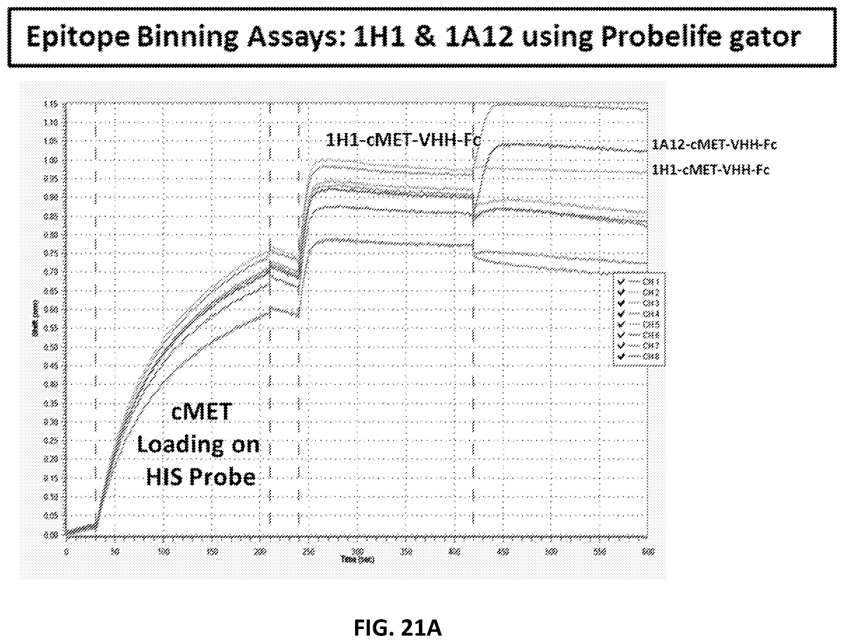

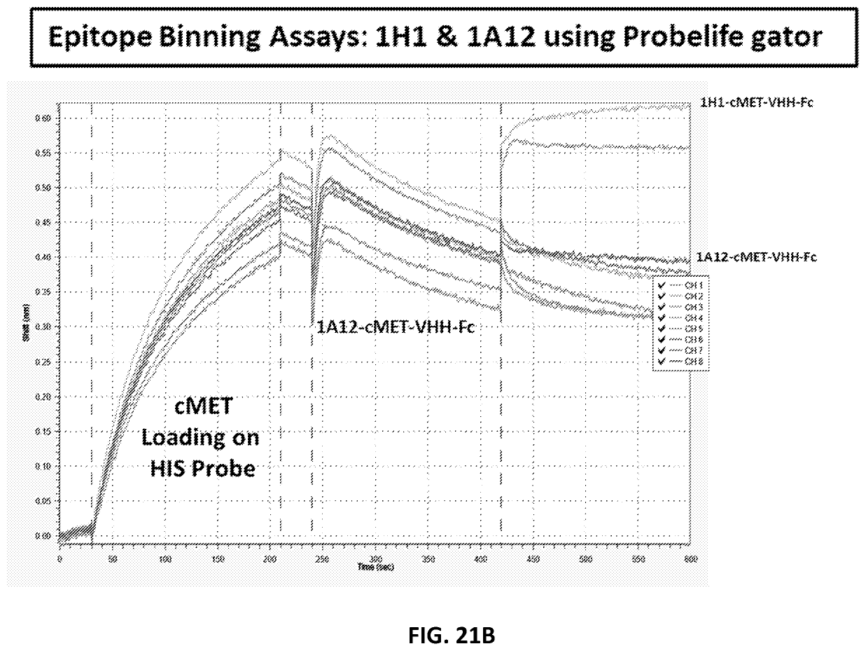

cMET VHH-Fc epitope binning analysis for cMET 1H1 and cMET 1A12 was performed using Bio-Layer interferometry (Probelife, Palo Alto, CA), according to the manufacturer's instructions (FIGS. 21A-21B). Briefly, a baseline was established using HIS probes in Quantitation (Q) buffer (a phosphate buffered saline solution with bovine serum albumin to decrease non-specific and background signal). The antigen (cMET-HIS tagged) to the HIS probe was added in 5 μg/ml cMET solution in Q buffer. HIS probes in Q buffer were used to return to the baseline. Subsequently, the association of the first antibody was done through HIS Probes in 15 μg/ml of antibody diluted in Q buffer. Finally, the secondary antibody was associated through HIS Probes in 5 μg/ml of antibody diluted in Q buffer. The results showed that the 1H1 and 1A12 VHHs may bind to different epitopes of cMET. Further, the binding of 1H1 VHH at its cMET epitope can further expose the 1A12 epitope, thereby increasing the binding affinity of the 1A12 VHH to cMet (FIG. 21A).

### Example 20. Construction of the Tri-Specific Ab

As shown in FIG. 18B, a trispecific antibody was prepared using the anti-EGFR IgG as the backbone. Two separate VHH domains which bind to two different epitopes of cMET were linked to the heavy chain variable region (VH) via a linker peptide to the two arms of the anti-EGFR IgG. The trispecific antibody is named as 1H1cMET/1A12cMET/EGFR(WT), when the 1H1 VHH (SEQ ID NO: 84) is linked to one arm of the anti-EGFR IgG (VH: SEQ ID NO: 23; VL: SEQ ID NO: 24), and the 1A12 VHH (SEQ ID NO: 20) is linked to the other arm of the anti-EGFR IgG. As discussed above, 1H1 and 1A12 VHHs bind to different epitopes of cMET. The binding of 1H1 VHH at its cMET epitope can further expose the 1A12 epitope, thereby increasing the binding affinity of the 1A12 VHH to cMet. As the expression of cMET is relatively small as compared to EGFR, the trispecific antibody 1H1cMET/1A12cMET/EGFR(WT) first binds to EGFR on cells. The binding of the trispecific antibody to EGFR brings 1H1 VHH closer to cMET on the cells. The binding of 1H1 VHH at its cMET epitope can further increase the exposure of the 1A12 epitope, thereby increasing the binding affinity of the trispecific antibody to the tumor cells.

### Example 21. Comparison of the Binding Capacities of 1H1cMET/1A12cMET/EGFR(WT) and JNJ372 Analog to Various NSCLC Cells

To determine the binding capacities of 1H1cMET/1A12cMET/EGFR(WT) and JNJ372 analog to various cellular models. The cells included H1975, H1975.HGF (H1975 transfected cells over expressing HGF), and the EGFR mutant C797S-BaF3 cells. Briefly, cells were diluted in FACS buffer and added to a 96-well V-bottom non-treated plate (5×10⁴ cells/100 μl/well). Serial dilutions of 1H1cMET/1A12cMET/EGFR(WT), JNJ372 analog and a control antibody from 2.04×10⁻⁴ nM to 400 nM in 100 μl volume media were added to different wells, respectively, and incubated for 60 minutes at room temperature (RT). The wells were then washed four times with PBS. Secondary Ab (Goat-anti-human Fc-FITC) in FACS buffer (1:200 dilution) was added to wells and incubated at RT for 30 minutes. The wells were washed three times with FACS buffer. The cells were detached with Accutase™ cell detachment solution and analyzed using FACS.

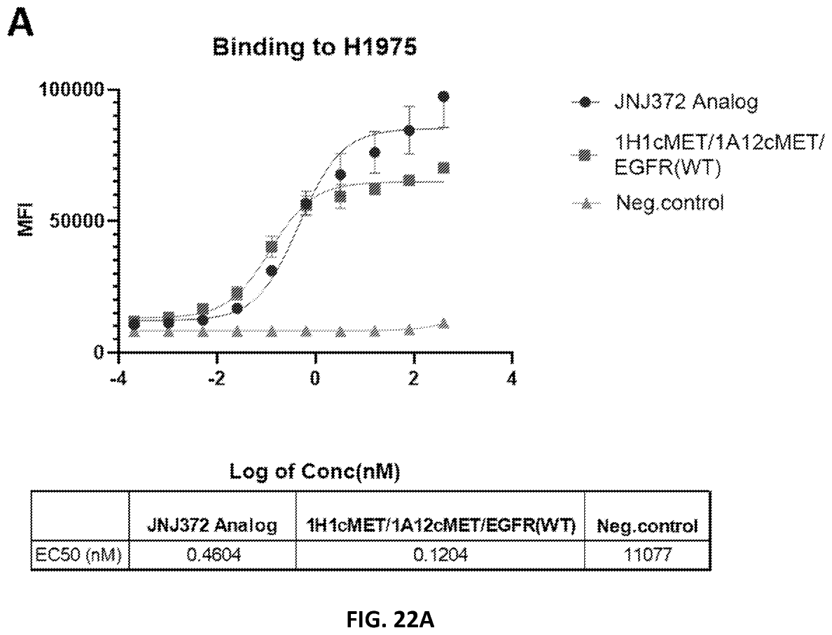

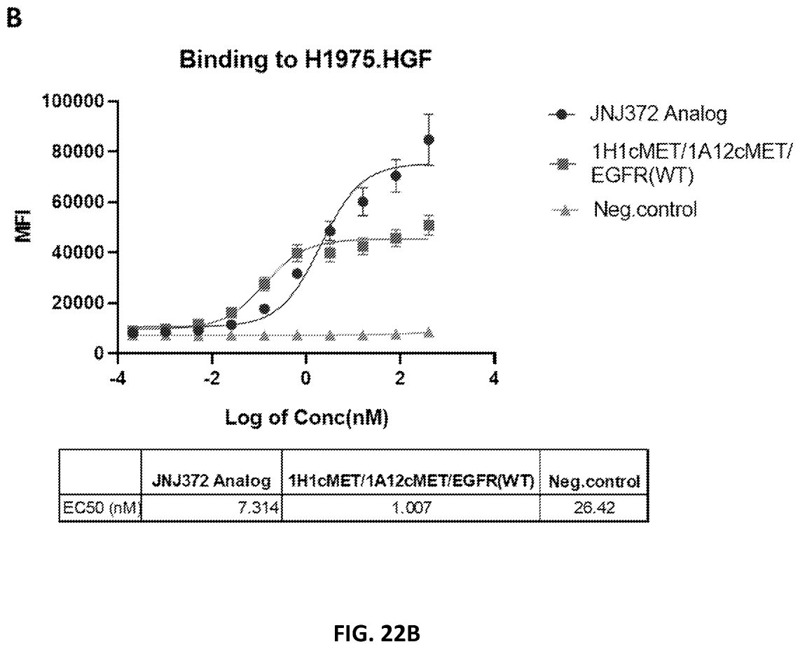

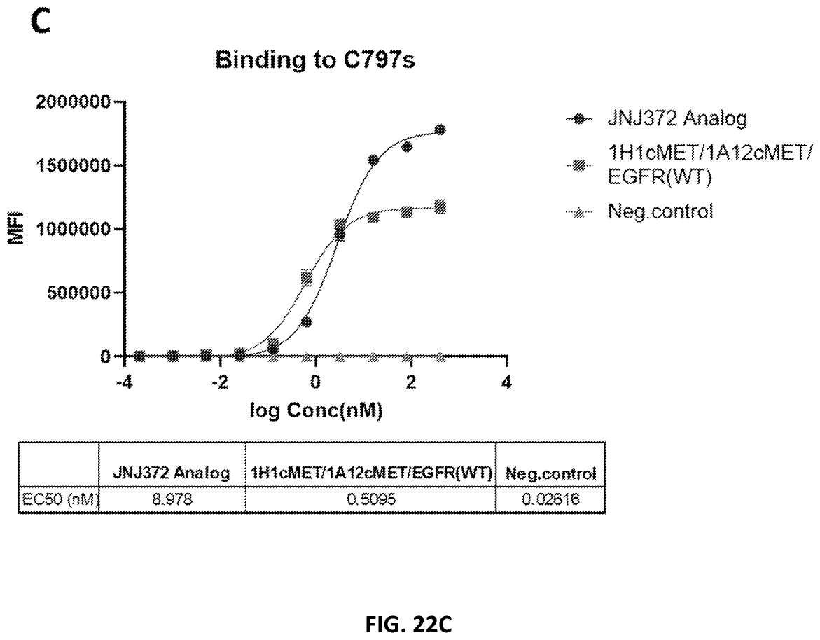

As shown in FIGS. 22A-22C, binding of the trispecific antibody 1H1cMET/1A12cMET/EGFR(WT) to NSCLC cells was better than that of JNJ372 analog.

### Example 22. Competitive Binding Assay to Determine EGFR/EGF Blocking Using JNJ372 Analog and 1H1cMET/1A12cMET/EGFR(WT)

Flow cytometry assays were used to determine whether the trispecific antibody 1H1cMET/1A12cMET/EGFR(WT) can block the interaction between EGF and EGFR. Biotinylated form of EGF was procured for this assay. NSCLC cells (e.g., H1975, H1975.HGF, or the EGFR mutant C797S-BaF3 cells) were plated in a 96-well plate (3×10⁵ cells per/well) in RPMI1640 media supplemented with 10% FBS and cultured overnight. The cells were washed three times with 1×PBS the next morning. After washing, the desired concentration of the antibodies (2×) in 50 μl volume was added (2.04×10⁻⁴ nM to 400 nM). Afterwards (without washing), 50 μl of biotinylated EGF (effective concentration 2 μg/ml; working concentration 4 g/ml) was added to each well, followed by an incubation for 45 minutes. The cells were subsequently washed with FACS Buffer twice and one time with Dulbecco's phosphate buffered saline (DPBS). 50 μl of Accutase™ cell detachment solution was added to each well and the plate was incubated at 37° C. for 5-7 minutes. The Accutase™ cell detachment solution was neutralized by adding 50 μl of media or FACS buffer. The contents of the wells were carefully transferred into a V-bottomed 96-well plate. The cells were washed with FACS buffer three times. Secondary antibody (Strepavidin Conjugate-PE; 1:500 dilution) was added to each well of the plate, and the plate was then incubated for 30-45 minutes. After the incubation, the plate was washed three times with FACS buffer. The cells were finally resuspended in FACS buffer and analyzed in a flow cytometer to quantify the EGF/EGFR blocking by the test samples.

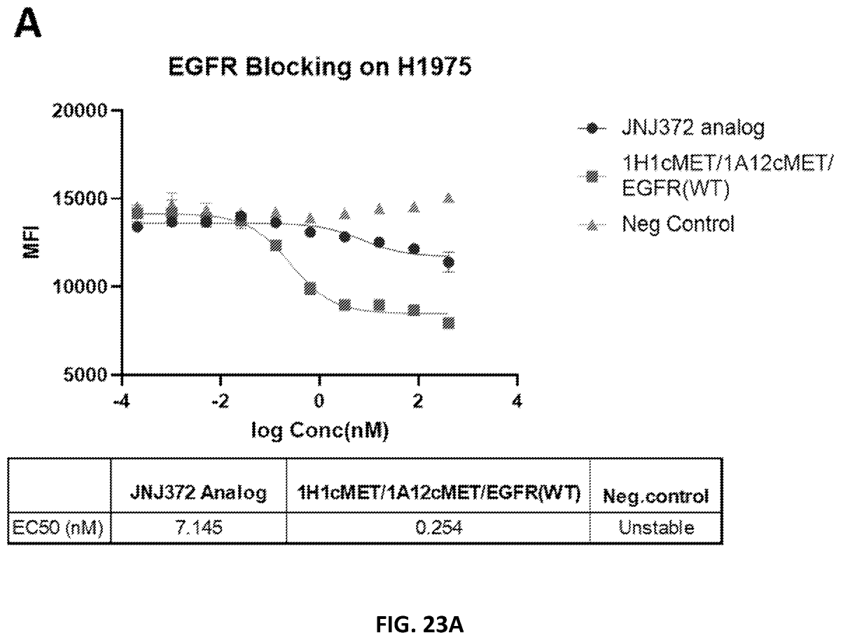

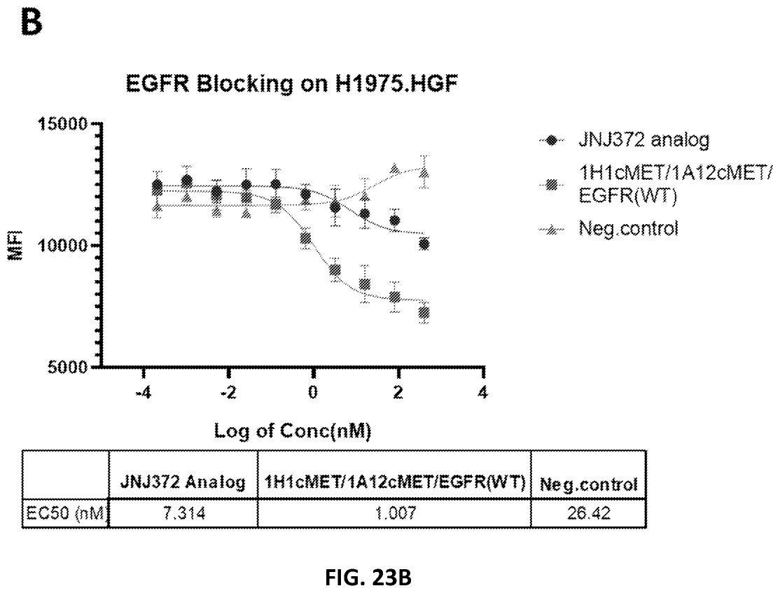

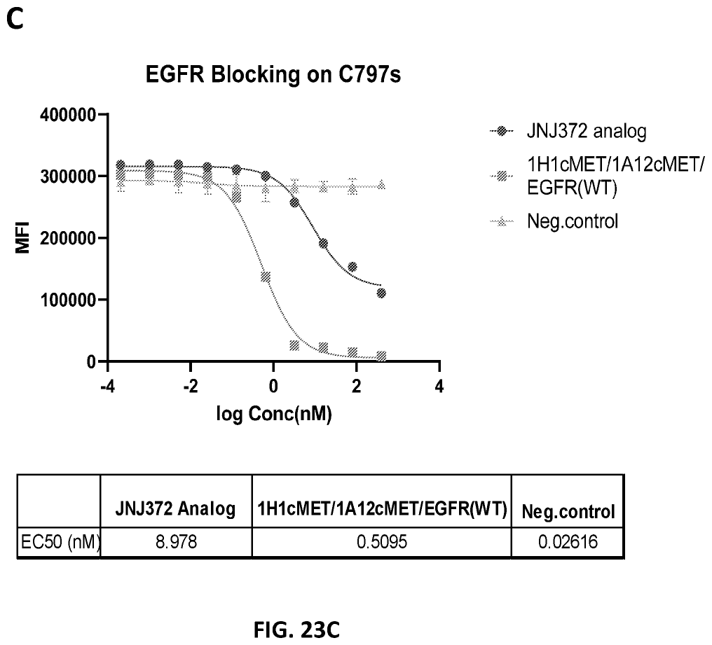

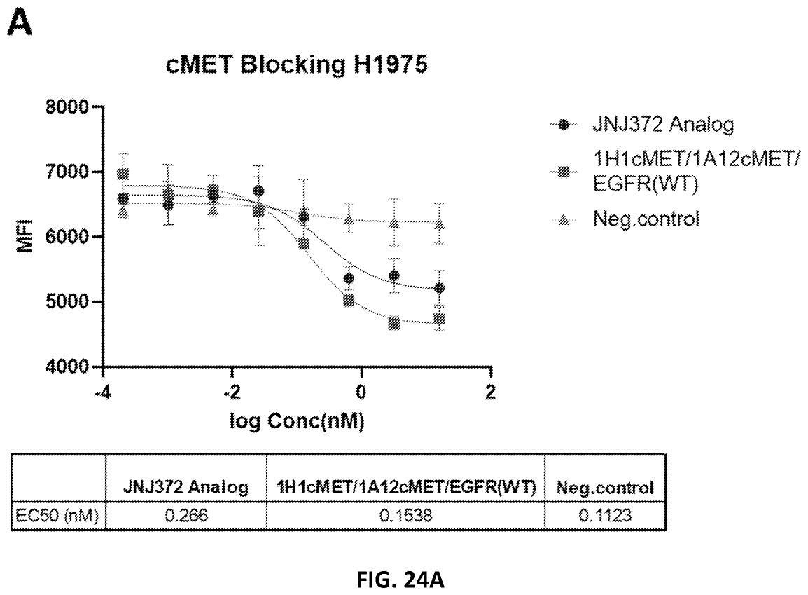

As shown in FIGS. 23A-23C, 1H1cMET/1A12cMET/EGFR(WT) showed significantly better EGF/EGFR blocking capabilities than JNJ372 analog in different cellular models.

### Example 23. Competitive Binding Assay to Determine cMET/HGF Blocking Using JNJ372 Analog and 1H1cMET/1A12cMET/EGFR(WT)

Flow cytometry assays were used to determine whether the trispecific antibody 1H1cMET/1A12cMET/EGFR(WT) can block the interaction between HGF (Hepatocyte growth factor) and cMET. HGF was biotinylated using a standard biotinylation kit (Pierce, Invitrogen). NSCLC cells (H1975 or H1975.HGF) were plated in a 96-well plate (3×10⁵ cells/well) in RPMI1640 media supplemented with 10% FBS and cultured overnight. The cells were washed three times with 1×PBS the next morning. After washing, the desired concentration of the antibodies (2×) in 50 μl volume was added (2.04×10⁻⁴ nM to 400 nM). Afterwards (without washing), 50 μl of biotinylated HGF (effective concentration 5 nM) was added to each well, followed by an incubation for 45 minutes. The cells were subsequently washed with FACS Buffer twice and one time with DPBS (Dulbecco's Phosphate Buffered Saline). 50 μl of Accutase™ cell detachment solution was added to each well and the plate was incubated at 37° C. for 5-7 minutes. The Accutase™ cell detachment solution was neutralized by adding 50 μl of media or FACS Buffer. The contents of the wells were carefully transferred into a V-bottomed 96-well plate. The cells were washed with FACS buffer three times. Secondary antibody (Strepavidin Conjugate-PE; 1:500 dilution) was added to each well of the plate, and the plate was incubated for 30-45 minutes. After the incubation, the plate was washed three times with FACS buffer. The cells were finally resuspended in FACS buffer and analyzed in a flow cytometer to quantify the EGF/EGFR blocking by the test samples.

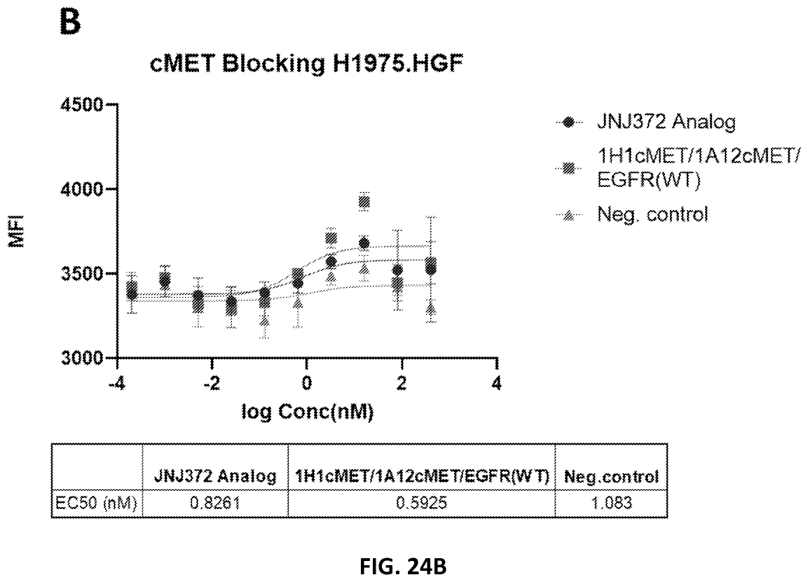

As shown in FIGS. 24A-24B, 1H1cMET/1A12cMET/EGFR(WT) showed better HGF/cMET blocking capabilities than JNJ372 analog in H1975.

### Example 24. 1H1cMET/1A12cMET/EGFR(WT) Modulates cMET and EGFR Downstream Signaling by Reducing pMET and Degrading EGFR

H1975, H1975.HGF and C797S-BaF3 cells were used to study the effect on cMET and EGFR downstream signaling. NCI-H1975 cells represent human cancer cell lines with EGFR primary and secondary activating mutations, which confers resistance to EGFR TKIs, in addition to MET gene amplification. In these cell lines the ligands of EGFR and c-Met, EGF and HGF respectively, induce phosphorylation of the EGFR and c-Met receptors which stimulates downstream signaling through the phosphorylation of ERK and Akt proteins. C797S represent cell lines with untreatable EGFR point mutation C797S.

To determine whether the trispecific antibody 1H1cMET/1A12cMET/EGFR(WT) can inhibit the activation of c-Met and EGFR and the phosphorylation of Akt and Erk, NCI-H1975 cells were plated on 6-well plates (3×10⁵ cells/well). On the subsequent morning, H1975 cells were treated with antibodies for 66 hours in complete medium, serum-starved in media with 0.5% FBS for 6 hours, and then stimulated with HGF for 15 minutes. H1975-HGF cells were treated with antibodies for 48 hours in complete medium, and then treated with fresh antibodies for additional 24 hours. C797S-BaF3 cells were treated with antibodies for 24 hours in complete medium.

The cells were washed twice with ice-cold PBS and then lysed in 0.25 ml of 1×cell lysis buffer (Cell Signaling Tech, Cat #9803) containing protease inhibitor (MedChemExpress, Cat #HY-K0010) and phosphatase inhibitor cocktails (Research Products International, Cat #P52100). Cell lysates were collected and centrifuged at 15000 g at 4° C. for 15 min. The cleared lysates were mixed with equal volume of 2×SDS sample buffer and boiled at 100° C. for 15 minutes. The samples were separated with SDS-PAGE, transferred to a PVDF membrane, blocked with 5% non-fat dry milk. The phosphorylation of EGFR, c-Met, Akt and Erk was detected with anti-phospho-EGFR (Y1173) (R&D Systems, Cat #AF1095), anti-phospho-c-Met (Cell Signaling Tech, Cat #AF2480), anti-phospho-Akt (Cell Signaling Tech, Cat #4060S), and anti-phospho-Erk (Cell Signaling Tech; Cat #4377S) antibodies, respectively. The total EGFR, c-Met, Akt and Erk were detected with anti-EGFR (BioLegend, Cat #617502), anti-c-Met (Cell Signaling Tech, Cat #3148S), anti-Akt (Cell Signaling Tech, Cat #2920S) and anti-Erk (Cell Signaling Tech, Cat #9107S) antibodies, respectively.

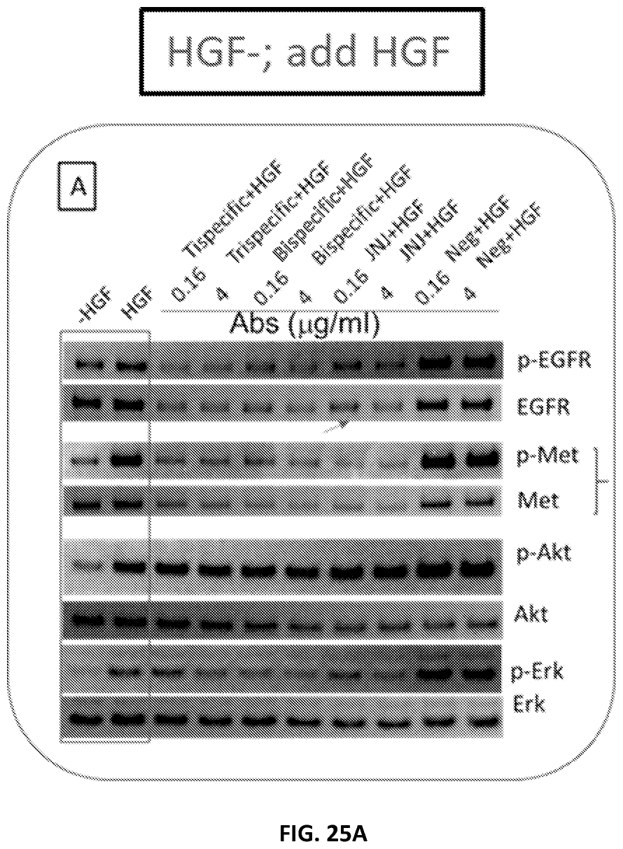

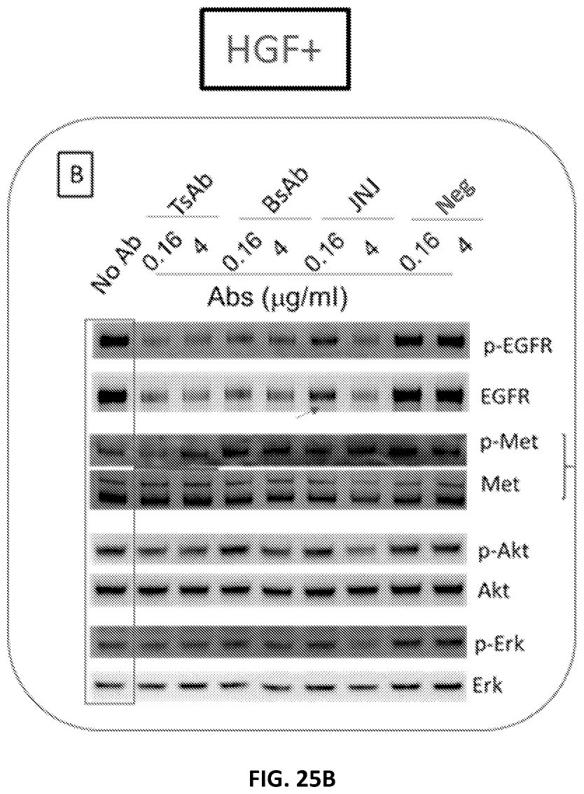

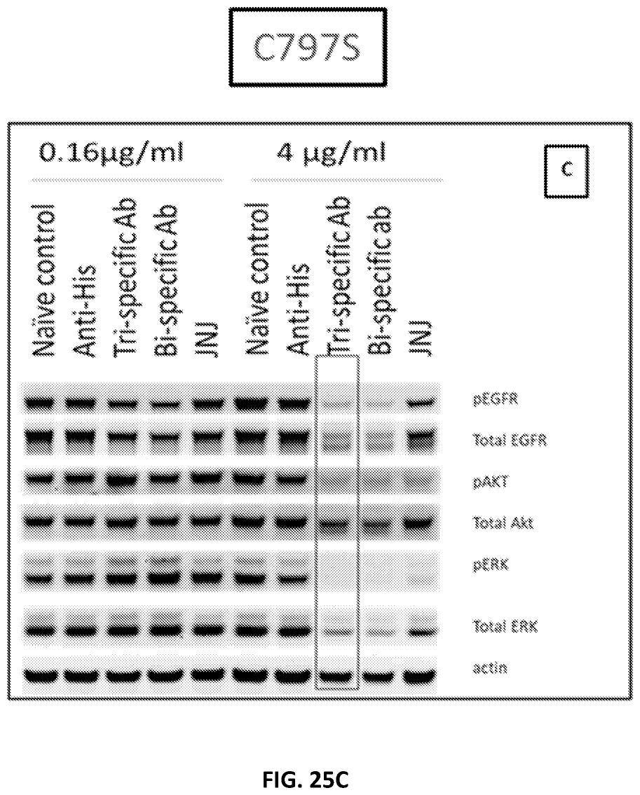

As shown in FIG. 25A. 1H1cMET/1A12cMET/EGFR(WT) was more effective than JNJ372 analog in degrading EGFR in HGF-treated H1975 cells. Similarly, in HGF-expressing H1975 cells, the trispecific antibody was more effective than JNJ372 analog in mediating EGFR degradation and inhibiting p-MET. (FIG. 25B). In addition, it induced dramatic degradation of EGFR and its downstream molecules (Akt and Erk) (FIG. 25C). These results suggest that 1H1cMET/1A12cMET/EGFR(WT) is more effective than JNJ372 analog in inhibiting cMet/EGFR signaling pathways.

### Example 25. 1H1cMET/1A12cMET/EGFR(WT) Inhibits Cellular Proliferation of NSCLC Cell Lines

Binding of cMET and EGFR to their respective ligands leads to cell proliferation and survival. This is elicited in response to the activation of downstream pathways from cMET and EGFR ligand binding. To assess this, different cancer cells (H1975, H1975.HGF and C797S-BaF3) were plated in a 96-well plate (3×10⁵ cells per/well) in RPMI1640 media supplemented with 10% FBS and cultured at 37° C. Subsequently, the cells were treated with a series of concentrations of 1H1cMET/1A12cMET/EGFR(WT) and the JNJ372 analog (2.04×10⁻⁴ nM to 400 nM) and kept in the incubator at 37° C. for 72 hours. Cell-Titer Glo (Promega, Madison WI) kit was used to assess the effect on proliferation according to the manufacturer's instructions through detection of luminescence.

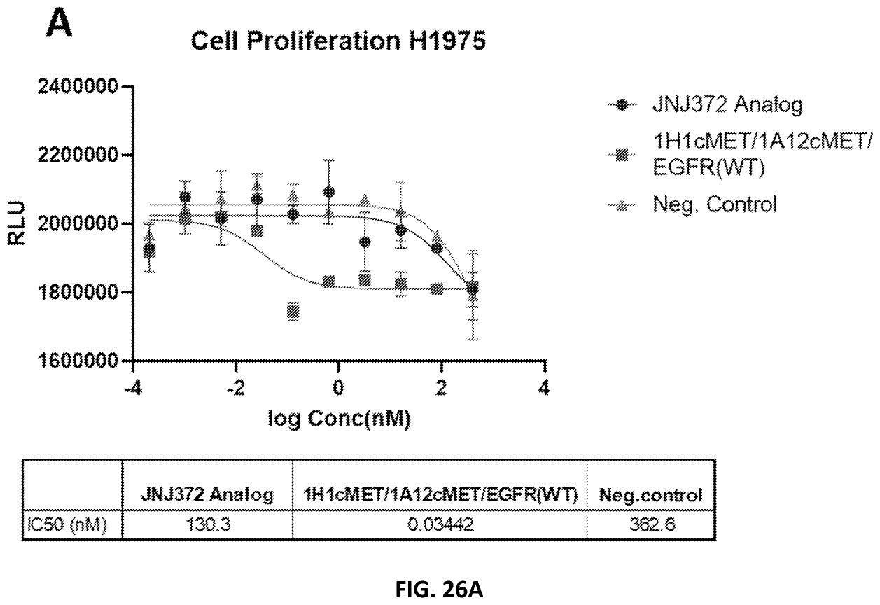

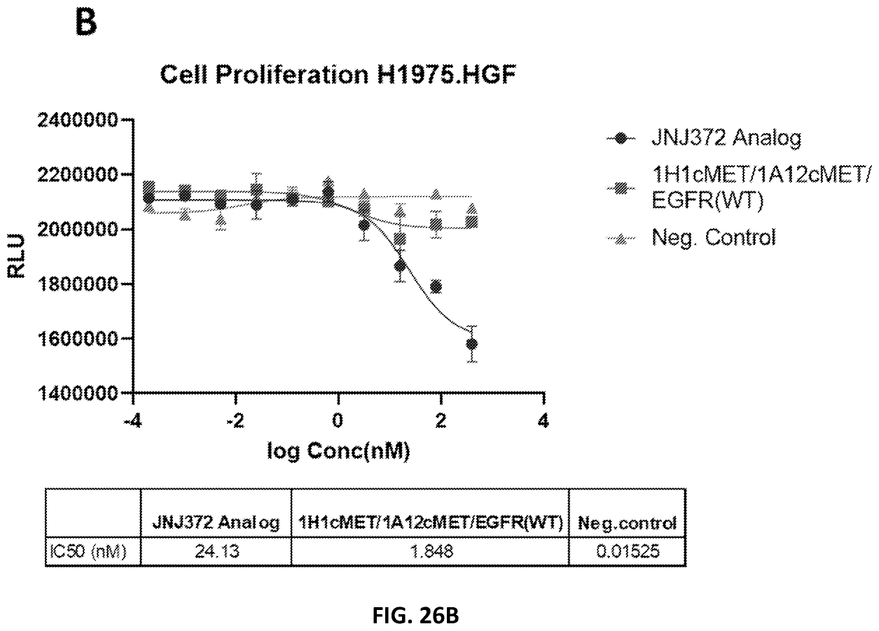

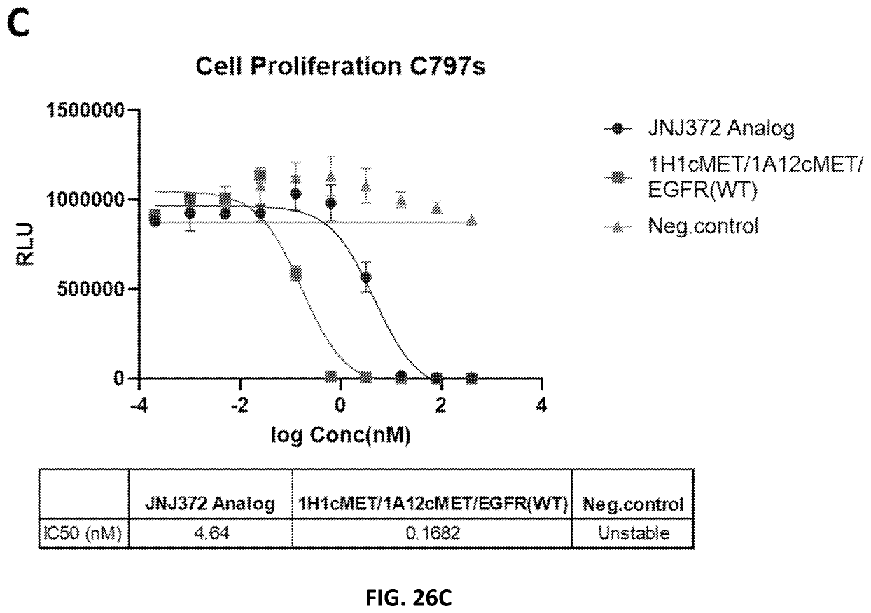

As shown in FIGS. 26A-26C, 1H1cMET/1A12cMET/EGFR(WT) was more effective than JNJ372 analog in inhibiting NSCLC cancer cell proliferation.

### Example 26. Examination of the Internalization of JNJ372 Analog and 1H1cMET/1A12cMET/EGFR(WT) Antibodies

To examine the internalization of JNJ372 analog and 1H1cMET/1A12cMET/EGFR(WT) antibodies, different cell lines were used (H1975, H1975.HGF, C797S-BaF3 and the EGFR insertion mutant D770-BaF3). The cells were plated in a 96-well plate (3×10⁴ cells/well) in 100 μL of RPMI 1640 media (supplemented with 10% FBS) and incubated at 37° C. and 5% CO₂. The adherent cells were incubated overnight whereas the suspension cells (C797S-BaF3 and D770-BaF3) were plated on the day of treatment. Subsequently, the cells were treated with a series of concentrations of 1H1cMET/1A12cMET/EGFR(WT) and the JNJ372 analog (2.04×10⁻⁴ nM to 400 nM) in 50 μL of RPMI 1640 media (supplemented with 10% FBS) and incubated for 20-30 minutes at 37° C., 5% CO₂. Afterwards, pHrodo™-labelled secondary Ab (AffiniPure Goat Anti-Human IgG, Fc7; Cat #109005008) (50 μl, 10 μg/ml) was added to cells and incubated for 20 hours at 37° C., 5% CO₂. To quantify the internalization of the receptors the adherent cells were detached using the Accutase™ cell detachment solution whereas the suspension cells were used directly. The cells were washed four times with FACS buffer, finally resuspended in the same buffer and analyzed by FACS using the PE channel.

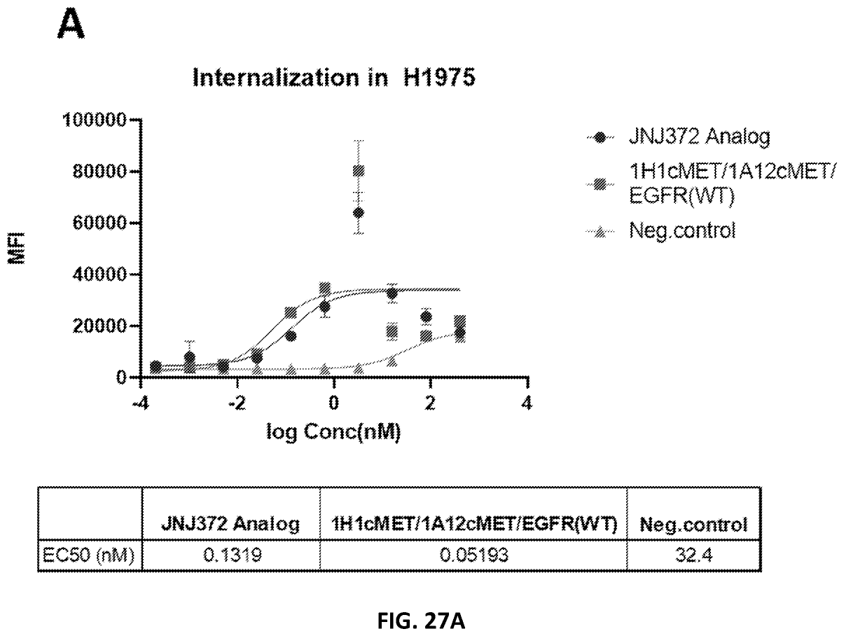

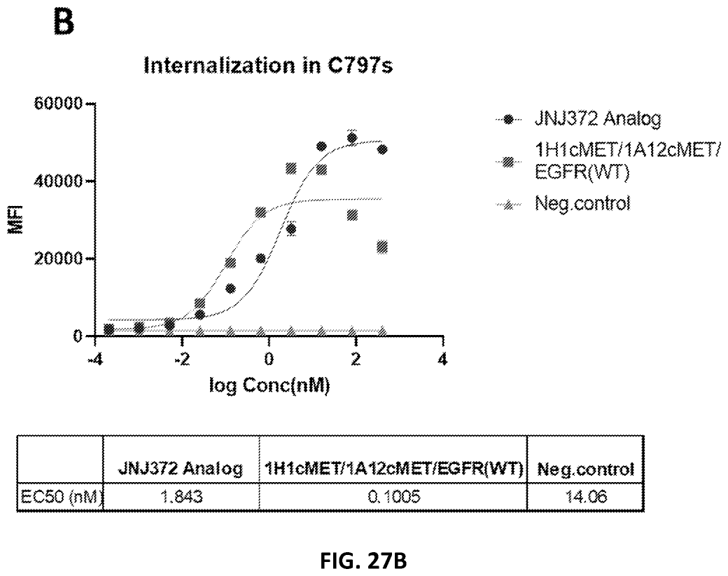

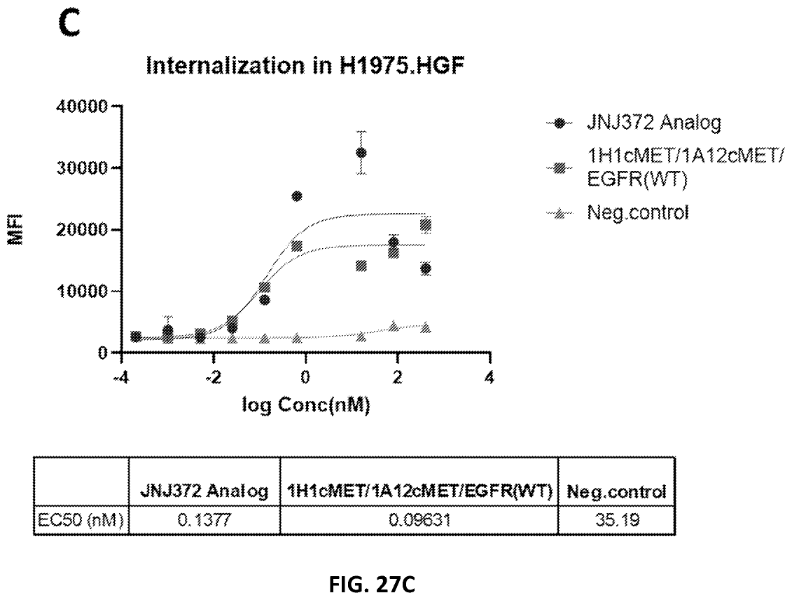

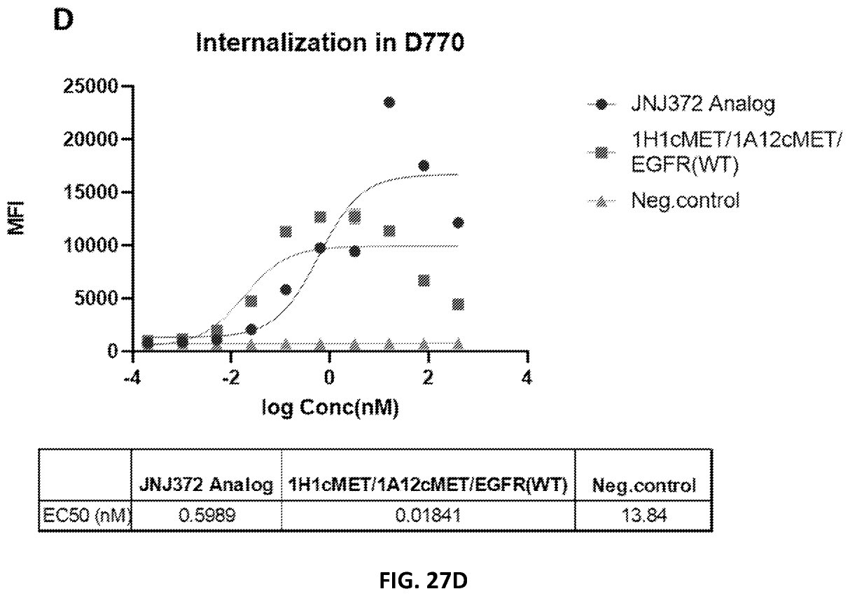

As shown in FIGS. 27A-27D, 1H1cMET/1A12cMET/EGFR(WT) was more effective than JNJ372 analog in antibody internalization in most NSCLC cells.

### Example 27. Determination of Effector Functions for 1H1cMET/1A12cMET/EGFR(WT) in Comparison to JNJ372 Analog

To determine the effector functions of 1H1cMET/1A12cMET/EGFR(WT) in comparison to JNJ372 analog, ADCC (antibody-dependent cellular cytotoxicity) assays were performed as described below. Specifically, H1975.GFP.Luc and H1975.GFP.Luc that stably expressing HGF (H1975.GFP.Luc.HGF) were used. The ratio of target (cancer cells) to effector (PBMCs) was 1:10, i.e., 9000 cells of target cells mixed with 90,000 cells of effector cells. The cancer cells were plated together with PBMCs and the antibodies in a total volume of 100 μl in a 96-well flat bottom plate. The plate was incubated for 24 hours at 37° C. After 24 hours, the supernatant was collected and transferred to a 96-well V-bottom plate. The cells in the flat bottom plate were washed with PBS. The Accutase™ cell detachment solution was used to disassociate the cells and FBS-containing RPMI medium was used to neutralize the effect of Accutase™ Subsequently, the cells were transferred from the flat bottom plate to a v-bottom plate. The cells with the supernatant were centrifuged and washed with FACS buffer three times. After the final wash 7AAD (Invitrogen) was added to a final dilution of 1:50. The cells were incubated in the dark for 20 minutes and then the plate was run on a flow cytometer with PerCP channel used for detection of the fluorescence.

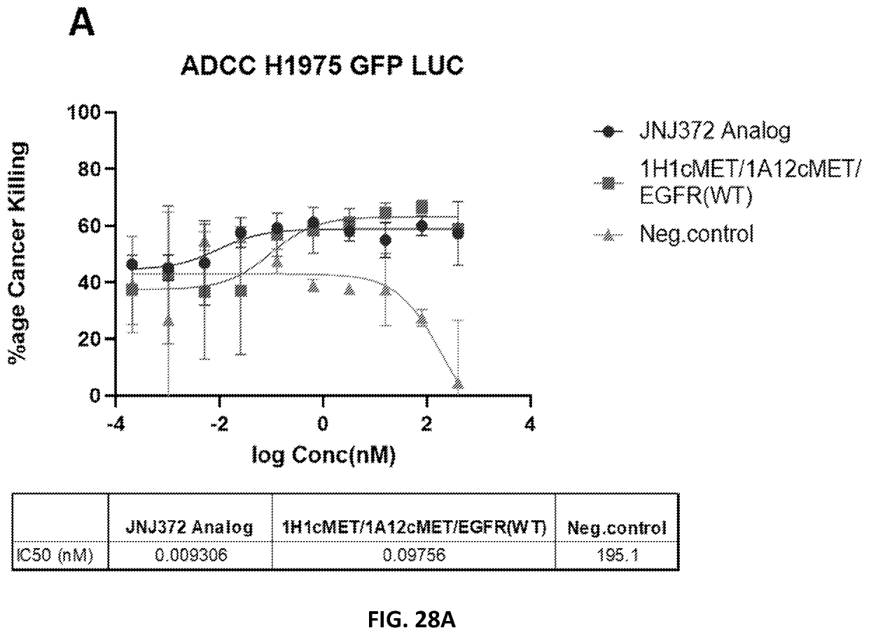

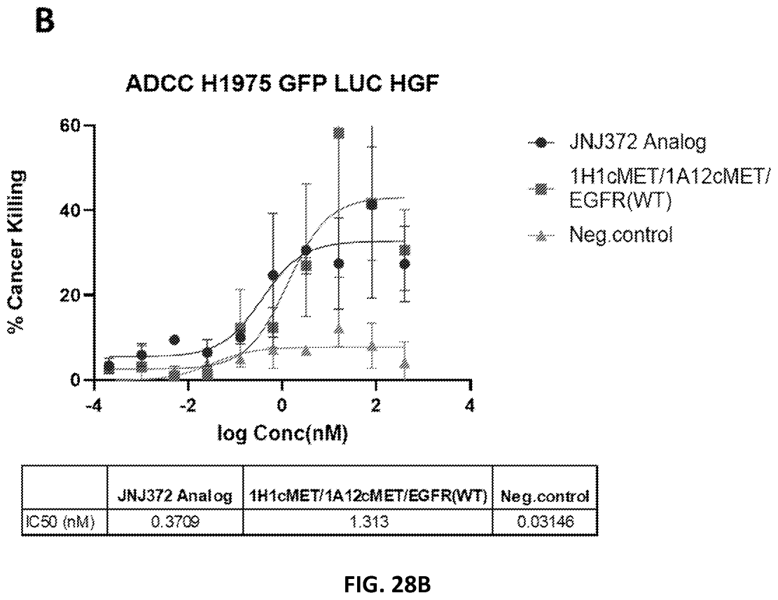

As shown in FIGS. 28A-28B, 1H1cMET/1A12cMET/EGFR(WT) was comparable to JNJ372 analog in ADCC in H1975 GFP.Luc cells whereas JNJ372 analog was better in H1975 GFP.Luc.HGF cells.

### Example 28. 4-Arm Efficacy Study in H1975+Balb/c NU/NU In Vivo Model

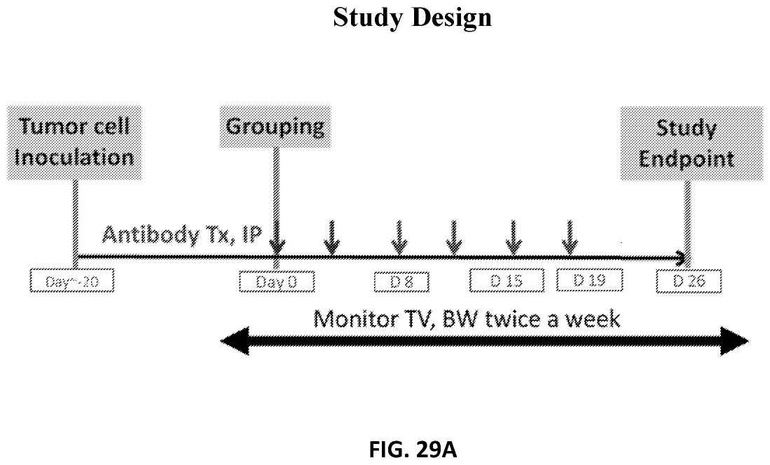

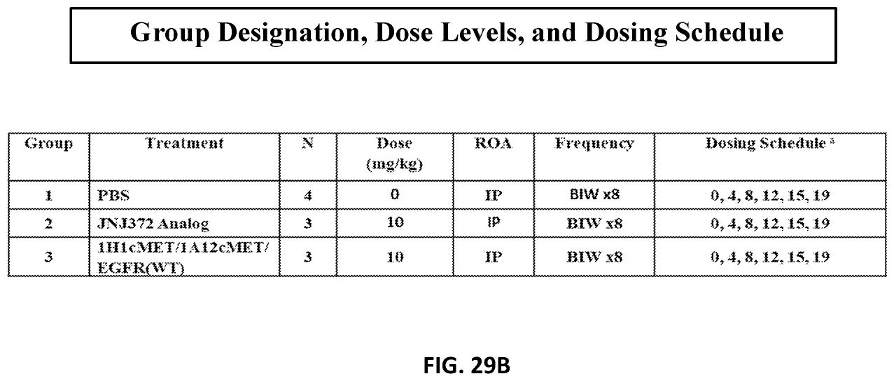

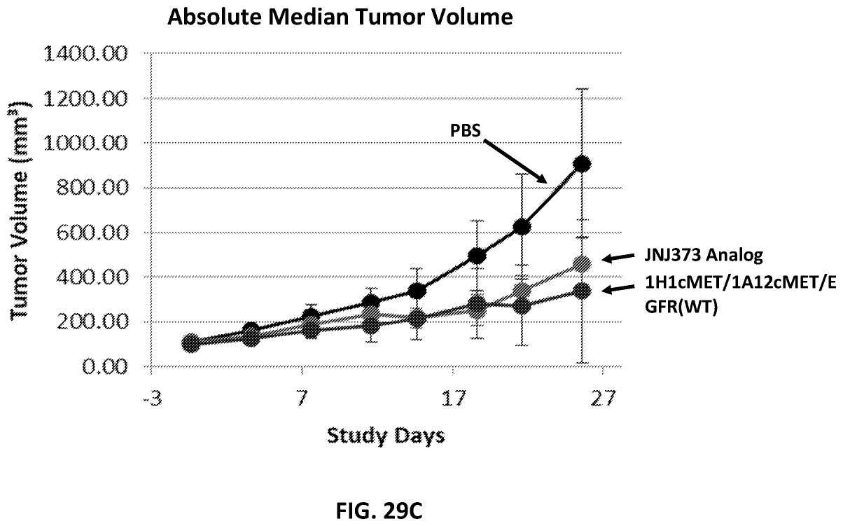

To assess the effect of the trispecific antibody 1H1cMET/1A12cMET/EGFR(WT) in reference to JNJ372 analog on tumor inhibition, a study on Balb/c NU/NU mice was performed as follows. Each mouse was subcutaneously injected with H1975 tumor cells (5.0×10⁶) in 0.1 mL 1:1 serum-free medium:matrigel mix in the right flank for tumor development. Tumor-bearing animals were randomly enrolled into study groups when mean tumor size reached approximately 100 mm³ (e.g., 75-125 mm³). Each group consisted of three to four mice. The day treatment was initiated is represented as Day 0 and animals in each group were treated as described in FIGS. 29A-29C.

The results indicates that 1H1cMET/1A12cMET/EGFR(WT) has a better cancer cell killing efficacy in H1975 engrafted Balb/c mouse model as compared to that of JNJ372 analog.

### Example 29. 4-Arm Efficacy Study in H1975-Luc-GFP-HGF+BALB/c NU/NU In Vivo Model

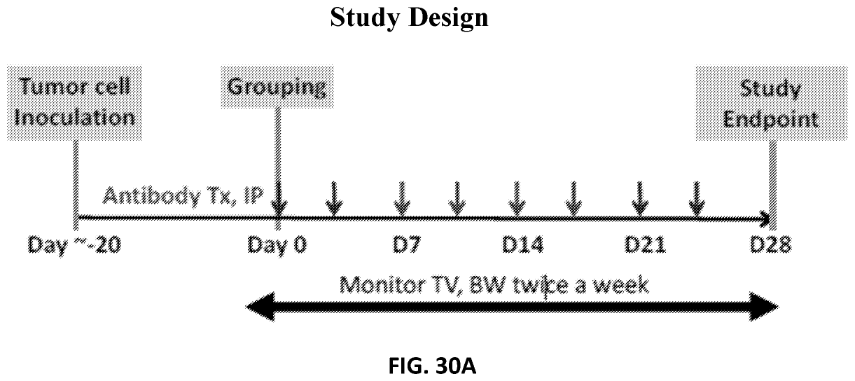

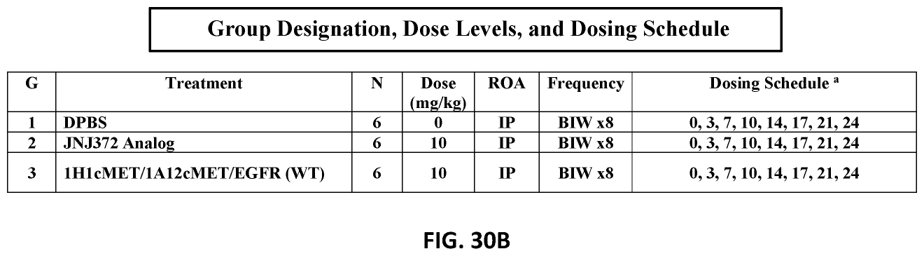

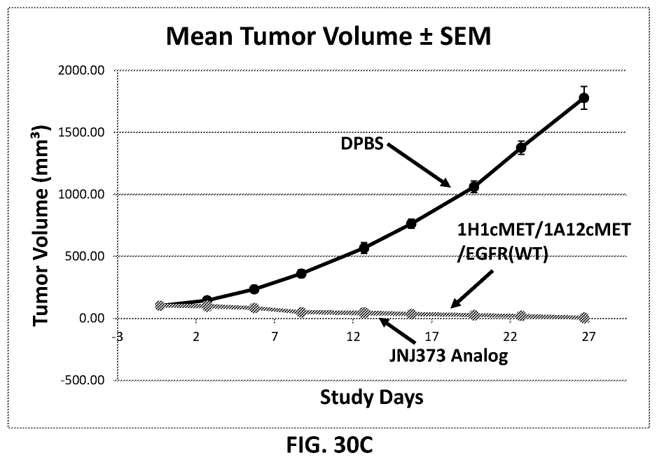

To assess the effect of the trispecific antibody 1H1cMET/1A12cMET/EGFR(WT) in reference to JNJ372 analog on tumor inhibition in an HGF overexpressing cell line, a study on Balb/c NU/NU mice was performed as follows. Each mouse was subcutaneously injected with H1975-Luc-GFP-HGF tumor cells (5.0×10⁶) in 0.1 mL 1:1 serum-free medium:matrigel mix in the right flank for tumor development. Tumor-bearing animals were randomly enrolled into study groups when mean tumor size reached approximately 100 mm³ (e.g., 75-125 mm³). Each group consisted of six mice. The day treatment was initiated is represented as Day 0 and animals in each group were treated as described in FIGS. 30A-30C.

The results indicate that 1H1cMET/1A12cMET/EGFR(WT) has similar cancer cell killing efficacy in H1975-Luc-GFP-HGF engrafted Balb/c mouse model as compared to that of JNJ372 analog.

### Example 30. Efficacy Evaluation of 1H1cMET/1A12cMET/EGFR(WT) in Female NOG Mice Bearing Cell Line C797S-BaF3 Subcutaneous Xenografts

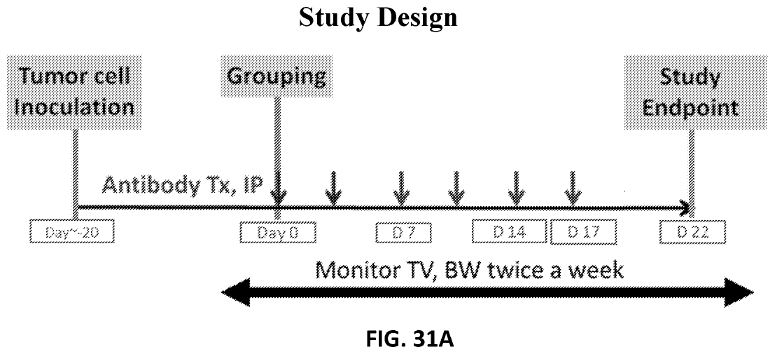

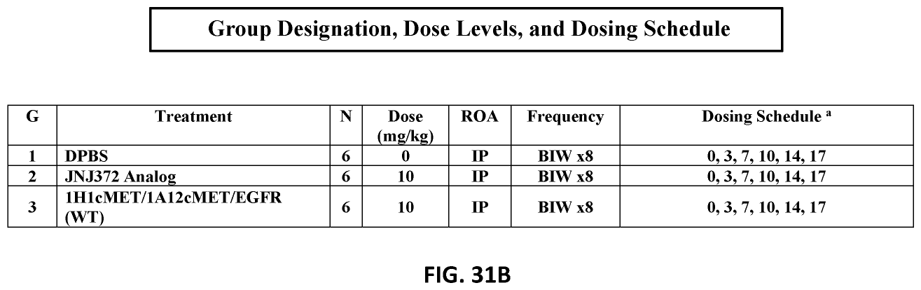

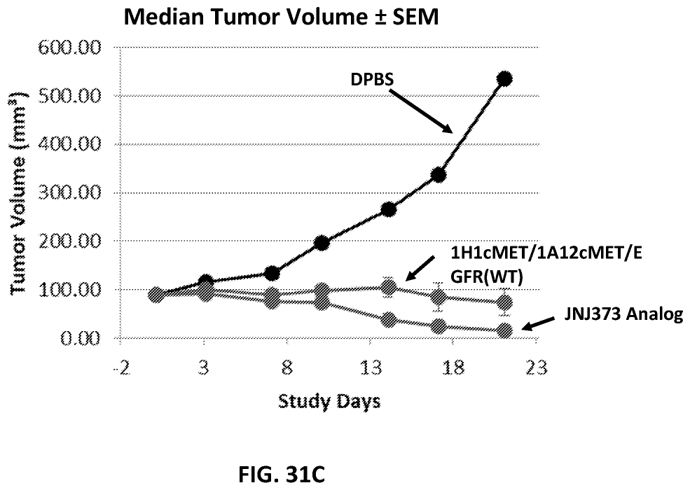

To assess the effect of the trispecific antibody 1H1cMET/1A12cMET/EGFR(WT) in reference to JNJ372 analog on tumor inhibition in an exon 20 point mutation cell line C797S-BaF3, a study on NOG mice was performed as follows. Each mouse was subcutaneously injected with C797S-BaF3 tumor cells (10.0×10⁶) in 0.1 mL 1:1 serum-free medium:matrigel mix in the right flank for tumor development. Tumor-bearing animals were randomly enrolled into study groups when mean tumor size reached approximately 100 mm³ (e.g., 75-125 mm³). Each group consisted of six mice. The day treatment was initiated is represented as Day 0 and animals in each group were treated as described in FIGS. 31A-31C.

The results indicate that 1H1cMET/1A12cMET/EGFR(WT) has comparable cancer cell killing efficacy in C797S-BaF3 engrafted NOG mouse model as compared to that of JNJ372 analog.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to cMET (tyrosine-protein kinase Met), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

2. The antibody or antigen-binding fragment thereof of claim 1, wherein the VHH comprises CDRs 1, 2, 3 with the amino acid sequences set forth in SEQ ID NOs: 1, 2, and 3, respectively.

3. The antibody or antigen-binding fragment thereof of claim 1, wherein the VHH comprises CDRs 1, 2, 3 with the amino acid sequences set forth in SEQ ID NOs: 4, 5, and 6, respectively.

4. The antibody or antigen-binding fragment thereof of claim 1, wherein the VHH comprises CDRs 1, 2, 3 with the amino acid sequences set forth in SEQ ID NOs: 7, 8, and 9, respectively.

5. The antibody or antigen-binding fragment thereof of claim 1, wherein the VHH comprises CDRs 1, 2, 3 with the amino acid sequences set forth in SEQ ID NOs: 10, 11, and 12, respectively.

6. An antibody or antigen-binding fragment thereof that binds to cMET comprising a heavy-chain antibody variable domain (VHH) comprising an amino acid sequence that is at least 80% identical to a selected VHH sequence, wherein the selected VHH sequence is selected from the group consisting of SEQ ID NOs: 19-22 and 77-84.

7. The antibody or antigen-binding fragment thereof of claim 6, wherein the VHH comprises the sequence of SEQ ID NO: 19, 77, 78, 79, 80, 81, or 84.

8. The antibody or antigen-binding fragment thereof of claim 6, wherein the VHH comprises the sequence of SEQ ID NO: 20.

9. The antibody or antigen-binding fragment thereof of claim 6, wherein the VHH comprises the sequence of SEQ ID NO: 21.

10. The antibody or antigen-binding fragment thereof of claim 6, wherein the VHH comprises the sequence of SEQ ID NO: 22, 82, or 83.

11. The antibody or antigen-binding fragment thereof of any one of claims 1-10, wherein the antibody or antigen-binding fragment specifically binds to cMET.

12. The antibody or antigen-binding fragment thereof of any one of claims 1-11, wherein the antibody or antigen-binding fragment is a humanized antibody or antigen-binding fragment thereof.

13. An antibody or antigen-binding fragment thereof comprising the VHH CDRs 1, 2, 3, of the antibody or antigen-binding fragment thereof of any one of claims 1-12.

14. The antibody or antigen-binding fragment thereof of any one of claims 1-13, wherein the antibody or antigen-binding fragment comprises a human IgG Fc.

15. The antibody or antigen-binding fragment thereof of any one of claims 1-14, wherein the antibody or antigen-binding fragment comprises two or more heavy-chain antibody variable domains.

16. An antibody or antigen-binding fragment thereof that cross-competes with the antibody or antigen-binding fragment thereof of any one of claims 1-15.

17. A multi-specific antibody or antigen-binding fragment thereof, comprising a first antigen-binding site that specifically binds to EGFR, and a second antigen-binding site that specifically binds to cMET.

18. The multi-specific antibody or antigen-binding fragment thereof of claim 17, wherein the first antigen-binding site comprises a heavy chain variable region (VH) and a light chain variable region (VL), wherein the VH and the VL associate with each other and specifically bind to EGFR, wherein
the heavy chain variable region (VH) comprising complementarity determining regions (CDRs) 1, 2, and 3, wherein the VH CDR1 region comprises an amino acid sequence that is at least 80% identical to SEQ ID NO: 13, the VH CDR2 region comprises an amino acid sequence that is at least 80% identical to SEQ ID NO: 14, and the VH CDR3 region comprises an amino acid sequence that is at least 80% identical to SEQ ID NO: 15; and
the light chain variable region (VL) comprising CDRs 1, 2, and 3, wherein the VL CDR1 region comprises an amino acid sequence that is at least 80% identical to SEQ ID NO: 16, the VL CDR2 region comprises an amino acid sequence that is at least 80% identical to SEQ ID NO: 17, and the VL CDR3 region comprises an amino acid sequence that is at least 80% identical to SEQ ID NO: 18.

19. The multi-specific antibody or antigen-binding fragment thereof of claim 18, wherein the heavy chain variable region (VH) comprises an amino acid sequence that is at least 90% identical to SEQ ID NO: 23, and a light chain variable region (VL) comprising an amino acid sequence that is at least 90% identical to SEQ ID NO: 24.

20. The multi-specific antibody or antigen-binding fragment thereof of any one of claims 17-19, wherein the second antigen-binding site specifically binds to cMET, and the second antigen-binding site comprises a first heavy-chain antibody variable domain (VHH1) comprising complementarity determining regions (CDRs) 1, 2, and 3, wherein the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence;
wherein the selected VHH1 CDRs 1, 2, and 3 amino acid sequences are one of the following:
(1) the selected VHH1 CDRs 1, 2, 3 amino acid sequences are set forth in SEQ ID NOs: 1, 2, and 3, respectively;
(2) the selected VHH1 CDRs 1, 2, 3 amino acid sequences are set forth in SEQ ID NOs: 4, 5, and 6, respectively;
(3) the selected VHH1 CDRs 1, 2, 3 amino acid sequences are set forth in SEQ ID NOs: 7, 8, and 9, respectively; and
(4) the selected VHH1 CDRs 1, 2, 3 amino acid sequences are set forth in SEQ ID NOs: 10, 11, and 12, respectively.

21. The multi-specific antibody or antigen-binding fragment thereof of claim 20, wherein the first heavy-chain antibody variable domain (VHH1) comprises an amino acid sequence that is at least 80% identical to a selected VHH sequence, wherein the selected VHH sequence is selected from the group consisting of SEQ ID NOs: 19-22 and 77-84.

22. The multi-specific antibody or antigen-binding fragment thereof of any one of claims 17-21, further comprising a third antigen-binding site that specifically binds to cMET.

23. The multi-specific antibody or antigen-binding fragment thereof of claim 22, wherein the third antigen-binding site specifically binds to cMET, and the third antigen-binding site comprises a second heavy-chain antibody variable domain (VHH2) comprising complementarity determining regions (CDRs) 1, 2, and 3, wherein the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence;
wherein the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

24. The multi-specific antibody or antigen-binding fragment thereof of claim 23, wherein the second heavy-chain antibody variable domain (VHH2) comprises an amino acid sequence that is at least 80% identical to a selected VHH2 sequence, wherein the selected VHH2 sequence is selected from the group consisting of SEQ ID NOs: 19-22 and 77-84.

25. The multi-specific antibody or antigen-binding fragment thereof of any one of claims 17-24, wherein the VH and the VL are linked by a linker peptide sequence to form an scFv.

26. The multi-specific antibody or antigen-binding fragment thereof of claim 25, wherein the linker peptide sequence comprises a sequence that is at least 80% identical to any one of SEQ ID NOs: 37-42.

27. A polypeptide complex, comprising
(a) a first polypeptide comprising from N-terminus to C-terminus: a first heavy-chain antibody variable domain (VHH), a first hinge region, a first Fc region;
(b) a second polypeptide comprising from N-terminus to C-terminus: a heavy chain variable region (VH), a second hinge region, and a second Fc region; and
(c) a third polypeptide comprising a light chain variable region (VL);
wherein the first VHH specifically binds to cMET, wherein the VH and the VL associate with each other and specifically bind to EGFR.

28. The polypeptide complex of claim 27, wherein the first hinge region and/or the second hinge region comprise a sequence that is at least 80% identical to any one of SEQ ID NOs: 25-29.

29. The polypeptide complex of claim 27 or 28, wherein the first Fc region and/or the second Fc region comprise a sequence that is at least 80% identical to SEQ ID NO: 30 or 31.

30. The polypeptide complex of any one of claims 27-29, wherein the first polypeptide comprises a sequence that is at least 80% identical to SEQ ID NO: 43 or 44; wherein the second polypeptide comprises a sequence that is at least 80% identical to SEQ ID NO: 45 or 46; and/or wherein the third polypeptide comprises a sequence that is at least 80% identical to SEQ ID NO: 47.

31. The polypeptide complex of any one of claims 27-30, wherein the first polypeptide further comprises a second VHH that specifically binds to cMET, wherein the second VHH is linked to the N-terminus of the first VHH via a linker peptide sequence.

32. The polypeptide complex of claim 31, wherein the linker peptide sequence comprises a sequence that is at least 80% identical to any one of SEQ ID NOs: 37-42.

33. The polypeptide complex of claim 31 or 32, wherein the first polypeptide comprises a sequence that is at least 80% identical to SEQ ID NO: 48 or 49; wherein the second polypeptide comprises a sequence that is at least 80% identical to SEQ ID NO: 50 or 51; and/or wherein the third polypeptide comprises a sequence that is at least 80% identical to SEQ ID NO: 52.

34. The polypeptide complex of any one of claims 27-33, wherein the first Fc region and/or the second Fc region comprises an aspartic acid (Asp) at position 239 according to EU numbering, and/or a glutamic acid (Glu) at position 332 according to EU numbering.

35. A polypeptide complex, comprising
(a) a first polypeptide comprising from N-terminus to C-terminus: a first heavy-chain antibody variable domain (VHH), a first hinge region, a first Fc region; and
(b) a second polypeptide comprising from N-terminus to C-terminus: a single-chain variable fragment (scFv), a second hinge region, and a second Fc region;
wherein the first VHH specifically binds to cMET; wherein the scFv comprises a heavy chain variable region (VH), a first linker peptide sequence, and a light chain variable region (VL); wherein the VH and the VL associate with each other and specifically bind to EGFR.

36. The polypeptide complex of claim 35, wherein the first linker peptide sequence comprises a sequence that is at least 80% identical to any one of SEQ ID NOs: 37-42.

37. The polypeptide complex of claim 35 or 36, wherein the first hinge region and/or the second hinge region comprise a sequence that is at least 80% identical to any one of SEQ ID NOs: 25-29.

38. The polypeptide complex of any one of claims 35-37, wherein the first Fc region and/or the second Fc region comprise a sequence that is at least 80% identical to SEQ ID NO: 30 or 31.

39. The polypeptide complex of any one of claims 35-38, wherein the first polypeptide comprises a sequence that is at least 80% identical to SEQ ID NO: 53 or 54; and/or wherein the second polypeptide comprises a sequence that is at least 80% identical to SEQ ID NO: 55 or 56.

40. The polypeptide complex of any one of claims 35-39, wherein the first polypeptide further comprises a second VHH that specifically binds to cMET, wherein the second VHH is linked to the N-terminus of the first VHH via a second linker peptide sequence.

41. The polypeptide complex of claim 40, wherein the second linker peptide sequence comprises a sequence that is at least 80% identical to any one of SEQ ID NOs: 37-42.

42. The polypeptide complex of claim 40 or 41, wherein the first polypeptide comprises a sequence that is at least 80% identical to SEQ ID NO: 57 or 58; and/or wherein the second polypeptide comprises a sequence that is at least 80% identical to SEQ ID NO: 59 or 60.

43. The polypeptide complex of any one of claims 35-42, wherein the first Fc region and/or the second Fc region comprises an aspartic acid (Asp) at position 239 according to EU numbering, and/or a glutamic acid (Glu) at position 332 according to EU numbering.

44. A polypeptide complex, comprising
(a) a first polypeptide comprising from N-terminus to C-terminus: a first heavy-chain antibody variable domain (VHH1), a first linker peptide sequence, a first heavy chain variable region (VH1), a first hinge region, and a first Fc region;
(b) a second polypeptide comprising a first light chain variable region (VL1);
(c) a third polypeptide comprising from N-terminus to C-terminus: a second heavy-chain antibody variable domain (VHH2), a second linker peptide sequence, a second heavy chain variable region (VH2), a second hinge region, and a second Fc region; and
(d) a fourth polypeptide comprising a second light chain variable region (VL2);
wherein the VHH1 and the VHH2 specifically bind to cMET; wherein the VH1 and the VL1 associate with each other and specifically bind to EGFR; wherein the VH2 and the VL2 associate with each other and specifically bind to EGFR.

45. The polypeptide complex of claim 44, wherein the first linker peptide sequence and/or the second linker peptide sequence comprises a sequence that is at least 80% identical to any one of SEQ ID NOs: 37-42.

46. The polypeptide complex of claim 44 or 45, wherein the first hinge region and/or the second hinge regions comprise a sequence that is at least 80% identical to any one of SEQ ID NOs: 25-29.

47. The polypeptide complex of any one of claims 44-46, wherein sequences of the VHH1 and the VHH2 are identical.

48. The polypeptide complex of claim 47, wherein the first Fc region and/or the second Fc region comprise a sequence that is at least 80% identical to SEQ ID NO: 32.

49. The polypeptide complex of claim 47 or 48, wherein the first polypeptide comprises a sequence that is at least 80% identical to SEQ ID NO: 61 or 62; wherein the second polypeptide comprises a sequence that is at least 80% identical to SEQ ID NO: 63; wherein the third polypeptide comprises a sequence that is at least 80% identical to SEQ ID NO: 61 or 62; and/or wherein the fourth polypeptide comprises a sequence that is at least 80% identical to SEQ ID NO: 63.

50. The polypeptide complex of any one of claims 44-46, wherein sequences of the VHH1 and the VHH2 are different.

51. The polypeptide complex of claim 50, wherein the first Fc region and/or the second Fc region comprise a sequence that is at least 80% identical to SEQ ID NO: 30 or 31.

52. The polypeptide complex of claim 50 or 51, wherein
(1) the first polypeptide comprises a sequence that is at least 80% identical to SEQ ID NO: 64 or 65; wherein the second polypeptide comprises a sequence that is at least 80% identical to SEQ ID NO: 68; wherein the third polypeptide comprises a sequence that is at least 80% identical to SEQ ID NO: 66 or 67; and/or wherein the fourth polypeptide comprises a sequence that is at least 80% identical to SEQ ID NO: 68; or
(2) the first polypeptide comprises a sequence that is at least 80% identical to SEQ ID NO: 85; wherein the second polypeptide comprises a sequence that is at least 80% identical to SEQ ID NO: 68; wherein the third polypeptide comprises a sequence that is at least 80% identical to SEQ ID NO: 86; and/or wherein the fourth polypeptide comprises a sequence that is at least 80% identical to SEQ ID NO: 68.

53. The polypeptide complex of any one of claims 44-52, wherein the first Fc region and/or the second Fc region comprises an aspartic acid (Asp) at position 239 according to EU numbering, and/or a glutamic acid (Glu) at position 332 according to EU numbering.

54. A polypeptide complex, comprising
(a) a first polypeptide comprising from N-terminus to C-terminus: a first heavy chain variable region (VH1), a first hinge region, a first Fc region; a first linker peptide sequence, and a first heavy-chain antibody variable domain (VHH1);
(b) a second polypeptide comprising a first light chain variable region (VL1);
(c) a third polypeptide comprising from N-terminus to C-terminus: a second heavy chain variable region (VH2), a second hinge region, a second Fc region, a second linker peptide sequence, and a second heavy-chain antibody variable domain (VHH2); and
(d) a fourth polypeptide comprising a second light chain variable region (VL2);
wherein the VHH1 and/or the VHH2 specifically bind to cMET; wherein the VH1 and the VL1 associate with each other and specifically bind to EGFR; wherein the VH2 and the VL2 associate with each other and specifically bind to EGFR.

55. The polypeptide complex of claim 54, wherein the first linker peptide sequence and/or the second linker peptide sequence comprises a sequence that is at least 80% identical to any one of SEQ ID NOs: 37-42.

56. The polypeptide complex of claim 54 or 55, wherein the first hinge region and/or the second hinge regions comprise a sequence that is at least 80% identical to any one of SEQ ID NOs: 25-29.

57. The polypeptide complex of any one of claims 54-56, wherein sequences of the VHH1 and the VHH2 are identical.

58. The polypeptide complex of claim 57, wherein the first Fc region and/or the second Fc region comprise a sequence that is at least 80% identical to SEQ ID NO: 32.

59. The polypeptide complex of claim 57 or 58, wherein the first polypeptide comprises a sequence that is at least 80% identical to SEQ ID NO: 69 or 70; wherein the second polypeptide comprises a sequence that is at least 80% identical to SEQ ID NO: 71; wherein the third polypeptide comprises a sequence that is at least 80% identical to SEQ ID NO: 69 or 70; and/or wherein the fourth polypeptide comprises a sequence that is at least 80% identical to SEQ ID NO: 71.

60. The polypeptide complex of any one of claims 54-56, wherein sequences of the VHH1 and the VHH2 are different.

61. The polypeptide complex of claim 60, wherein the first Fc region and/or the second Fc region comprise a sequence that is at least 80% identical to SEQ ID NO: 30 or 31.

62. The polypeptide complex of claim 60 or 61, wherein the first polypeptide comprises a sequence that is at least 80% identical to SEQ ID NO: 72 or 73; wherein the second polypeptide comprises a sequence that is at least 80% identical to SEQ ID NO: 76; wherein the third polypeptide comprises a sequence that is at least 80% identical to SEQ ID NO: 74 or 75; and/or wherein the fourth polypeptide comprises a sequence that is at least 80% identical to SEQ ID NO: 76.

63. The polypeptide complex of any one of claims 54-62, wherein the first Fc region and/or the second Fc region comprises an aspartic acid (Asp) at position 239 according to EU numbering, and/or a glutamic acid (Glu) at position 332 according to EU numbering.

64. A polypeptide complex, comprising
(a) a first polypeptide comprising from N-terminus to C-terminus: a first heavy-chain antibody variable domain (VHH), a first hinge region, a first Fc region;
(b) a second polypeptide comprising from N-terminus to C-terminus: a heavy chain variable region (VH), a second hinge region, and a second Fc region; and
(c) a third polypeptide comprising a light chain variable region (VL);
wherein the first VHH specifically binds to cMET, wherein the VH and the VL associate with each other and specifically bind to EGFR; wherein the first polypeptide further comprises a second VHH that specifically binds to cMET, wherein the second VHH is linked to the N-terminus of the first VHH via a linker peptide sequence.

65. The polypeptide complex of claim 64, wherein the first hinge region and/or the second hinge region comprise a sequence that is at least 80% identical to any one of SEQ ID NOs: 25-29.

66. The polypeptide complex of claim 64 or 65, wherein the first Fc region and/or the second Fc region comprise a sequence that is at least 80% identical to SEQ ID NO: 30 or 31.

67. The polypeptide complex of any one of claims 64-66, wherein the linker peptide sequence comprises a sequence that is at least 80% identical to any one of SEQ ID NOs: 37-42.

68. The polypeptide complex of any one of claims 64-67, wherein the first polypeptide comprises a sequence that is at least 80% identical to SEQ ID NO: 48 or 49; wherein the second polypeptide comprises a sequence that is at least 80% identical to SEQ ID NO: 50 or 51; and/or wherein the third polypeptide comprises a sequence that is at least 80% identical to SEQ ID NO: 52.

69. The polypeptide complex of any one of claims 64-68, wherein the first Fc region and/or the second Fc region comprises an aspartic acid (Asp) at position 239 according to EU numbering, and/or a glutamic acid (Glu) at position 332 according to EU numbering.

70. A polypeptide complex, comprising
(a) a first polypeptide comprising from N-terminus to C-terminus: a first heavy-chain antibody variable domain (VHH), a first hinge region, a first Fc region; and
(b) a second polypeptide comprising from N-terminus to C-terminus: a single-chain variable fragment (scFv), a second hinge region, and a second Fc region;
wherein the first VHH specifically binds to cMET; wherein the scFv comprises a heavy chain variable region (VH), a first linker peptide sequence, and a light chain variable region (VL); wherein the VH and the VL associate with each other and specifically bind to EGFR; wherein the first polypeptide further comprises a second VHH that specifically binds to cMET, wherein the second VHH is linked to the N-terminus of the first VHH via a second linker peptide sequence.

71. The polypeptide complex of claim 70, wherein the first linker peptide sequence and/or the second linker peptide sequence comprise a sequence that is at least 80% identical to any one of SEQ ID NOs: 37-42.

72. The polypeptide complex of claim 70 or 71, wherein the first hinge region and/or the second hinge region comprise a sequence that is at least 80% identical to any one of SEQ ID NOs: 25-29.

73. The polypeptide complex of any one of claims 70-72, wherein the first Fc region and/or the second Fc region comprise a sequence that is at least 80% identical to SEQ ID NO: 30 or 31.

74. The polypeptide complex of any one of claims 70-73, wherein the first polypeptide comprises a sequence that is at least 80% identical to SEQ ID NO: 57 or 58; and/or wherein the second polypeptide comprises a sequence that is at least 80% identical to SEQ ID NO: 59 or 60.

75. The polypeptide complex of any one of claims 70-74, wherein the first Fc region and/or the second Fc region comprises an aspartic acid (Asp) at position 239 according to EU numbering, and/or a glutamic acid (Glu) at position 332 according to EU numbering.

76. A polypeptide complex, comprising
(a) a first polypeptide comprising from N-terminus to C-terminus: a first heavy-chain antibody variable domain (VHH1), a first linker peptide sequence, a first heavy chain variable region (VH1), a first hinge region, and a first Fc region;
(b) a second polypeptide comprising a first light chain variable region (VL1);
(c) a third polypeptide comprising from N-terminus to C-terminus: a second heavy-chain antibody variable domain (VHH2), a second linker peptide sequence, a second heavy chain variable region (VH2), a second hinge region, and a second Fc region; and
(d) a fourth polypeptide comprising a second light chain variable region (VL2);
wherein the VHH1 and the VHH2 specifically bind to cMET; wherein the VH1 and the VL1 associate with each other and specifically bind to EGFR; wherein the VH2 and the VL2 associate with each other and specifically bind to EGFR; wherein sequences of the VHH1 and the VHH2 are different.

77. The polypeptide complex of claim 76, wherein the first linker peptide sequence and/or the second linker peptide sequence comprise a sequence that is at least 80% identical to any one of SEQ ID NOs: 37-42.

78. The polypeptide complex of claim 76 or 77, wherein the first hinge region and/or the second hinge regions comprise a sequence that is at least 80% identical to any one of SEQ ID NOs: 25-29.

79. The polypeptide complex of any one of claims 76-78, wherein the first Fc region and/or the second Fc region comprise a sequence that is at least 80% identical to SEQ ID NO: 30 or 31.

80. The polypeptide complex of any one of claims 76-79, wherein
(1) the first polypeptide comprises a sequence that is at least 80% identical to SEQ ID NO: 64 or 65; wherein the second polypeptide comprises a sequence that is at least 80% identical to SEQ ID NO: 68; wherein the third polypeptide comprises a sequence that is at least 80% identical to SEQ ID NO: 66 or 67; and/or wherein the fourth polypeptide comprises a sequence that is at least 80% identical to SEQ ID NO: 68; or
(2) the first polypeptide comprises a sequence that is at least 80% identical to SEQ ID NO: 85; wherein the second polypeptide comprises a sequence that is at least 80% identical to SEQ ID NO: 68; wherein the third polypeptide comprises a sequence that is at least 80% identical to SEQ ID NO: 86; and/or wherein the fourth polypeptide comprises a sequence that is at least 80% identical to SEQ ID NO: 68.

81. The polypeptide complex of any one of claims 76-80, wherein the first Fc region and/or the second Fc region comprises an aspartic acid (Asp) at position 239 according to EU numbering, and/or a glutamic acid (Glu) at position 332 according to EU numbering.

82. A polypeptide complex, comprising
(a) a first polypeptide comprising from N-terminus to C-terminus: a first heavy chain variable region (VH1), a first hinge region, a first Fc region; a first linker peptide sequence, and a first heavy-chain antibody variable domain (VHH1);
(b) a second polypeptide comprising a first light chain variable region (VL1);
(c) a third polypeptide comprising from N-terminus to C-terminus: a second heavy chain variable region (VH2), a second hinge region, a second Fc region, a second linker peptide sequence, and a second heavy-chain antibody variable domain (VHH2); and
(d) a fourth polypeptide comprising a second light chain variable region (VL2);
wherein the VHH1 and/or the VHH2 specifically bind to cMET; wherein the VH1 and the VL1 associate with each other and specifically bind to EGFR; wherein the VH2 and the VL2 associate with each other and specifically bind to EGFR; wherein sequences of the VHH1 and the VHH2 are different.

83. The polypeptide complex of claim 82, wherein the first linker peptide sequence and/or the second linker peptide sequence comprises a sequence that is at least 80% identical to any one of SEQ ID NOs: 37-42.

84. The polypeptide complex of claim 82 or 83, wherein the first hinge region and/or the second hinge regions comprise a sequence that is at least 80% identical to any one of SEQ ID NOs: 25-29.

85. The polypeptide complex of any one of claims 82-84, wherein the first Fc region and/or the second Fc region comprise a sequence that is at least 80% identical to SEQ ID NO: 30 or 31.

86. The polypeptide complex of any one of claims 82-85, wherein the first polypeptide comprises a sequence that is at least 80% identical to SEQ ID NO: 72 or 73; wherein the second polypeptide comprises a sequence that is at least 80% identical to SEQ ID NO: 76; wherein the third polypeptide comprises a sequence that is at least 80% identical to SEQ ID NO: 74 or 75; and/or wherein the fourth polypeptide comprises a sequence that is at least 80% identical to SEQ ID NO: 76.

87. The polypeptide complex of any one of claims 82-86, wherein the first Fc region and/or the second Fc region comprises an aspartic acid (Asp) at position 239 according to EU numbering, and/or a glutamic acid (Glu) at position 332 according to EU numbering.

88. A nucleic acid comprising a polynucleotide encoding the antibody or antigen-binding fragment thereof of any one of claims 1-16, the multi-specific antibody or antigen-binding fragment thereof of any one of claims 17-26, or the polypeptide complex of any one of claims 27-87.

89. The nucleic acid of claim 88, wherein the nucleic acid is a DNA (e.g., cDNA) or RNA (e.g., mRNA).

90. A vector comprising one or more of the nucleic acids of claim 88 or 89.

91. A cell comprising the vector of claim 90.

92. The cell of claim 91, wherein the cell is a CHO cell.

93. A cell comprising one or more of the nucleic acids of claim 88 or 89.

94. A method of producing an antibody or an antigen-binding fragment thereof, the method comprising
(a) culturing the cell of any one of claims 91-93 under conditions sufficient for the cell to produce the antibody or the antigen-binding fragment; and
(b) collecting the antibody or the antigen-binding fragment produced by the cell.

95. An antibody-drug conjugate comprising the antibody or antigen-binding fragment thereof of any one of claims 1-16, the multi-specific antibody or antigen-binding fragment thereof of any one of claims 17-26, or the polypeptide complex of any one of claims 27-87, covalently bound to a therapeutic agent.

96. The antibody drug conjugate of claim 95, wherein the therapeutic agent is a cytotoxic or cytostatic agent.

97. A method of treating a subject having cancer, the method comprising administering a therapeutically effective amount of a composition comprising the antibody or antigen-binding fragment thereof of any one of claims 1-16, the multi-specific antibody or antigen-binding fragment thereof of any one of claims 17-26, the polypeptide complex of any one of claims 27-87, or the antibody-drug conjugate of claims 95 or 96, to the subject.

98. The method of claim 97, wherein the subject has a cancer expressing cMET.

99. The method of claim 97 or 98, wherein the subject has a cancer expressing EGFR.

100. The method of any one of claims 97-99, wherein the cancer is lung cancer.

101. The method of claim 100, wherein the cancer is non-small cell lung cancer (NSCLC).

102. A method of decreasing the rate of tumor growth, the method comprising contacting a tumor cell with an effective amount of a composition comprising the antibody or antigen-binding fragment thereof of any one of claims 1-16, the multi-specific antibody or antigen-binding fragment thereof of any one of claims 17-26, the polypeptide complex of any one of claims 27-87, or the antibody-drug conjugate of claims 95 or 96.

103. A method of killing a tumor cell, the method comprising contacting a tumor cell with an effective amount of a composition comprising the antibody or antigen-binding fragment thereof of any one of claims 1-16, the multi-specific antibody or antigen-binding fragment thereof of any one of claims 17-26, the polypeptide complex of any one of claims 27-87, or the antibody-drug conjugate of claims 95 or 96.

104. A method of killing a tumor cell, the method comprising contacting a tumor cell with an effective amount of a composition comprising the antibody or antigen-binding fragment thereof of any one of claims 1-16, the multi-specific antibody or antigen-binding fragment thereof of any one of claims 17-26, the polypeptide complex of any one of claims 27-87, or the antibody-drug conjugate of claims 95 or 96.

105. A pharmaceutical composition comprising the antibody or antigen-binding fragment thereof of any one of claims 1-16, the multi-specific antibody or antigen-binding fragment thereof of any one of claims 17-26, the polypeptide complex of any one of claims 27-87, and a pharmaceutically acceptable carrier.

106. A pharmaceutical composition comprising the antibody-drug conjugate of claims 95 or 96, and a pharmaceutically acceptable carrier.


SEQUENCE LISTING (86 sequences)

SEQ ID NO: 1 - protein, 10 aa, Artificial
  CDR sequence of cMET VHH
ASLFSINTMG

SEQ ID NO: 2 - protein, 16 aa, Artificial
  CDR sequence of cMET VHH
AISWSGGNYYATFARG

SEQ ID NO: 3 - protein, 17 aa, Artificial
  CDR sequence of cMET VHH
GADRWGTGSMNAPDYDY

SEQ ID NO: 4 - protein, 10 aa, Artificial
  CDR sequence of cMET VHH
GFTFNSYWMY

SEQ ID NO: 5 - protein, 17 aa, Artificial
  CDR sequence of cMET VHH
ATDTRGQNTYYTDSVKG

SEQ ID NO: 6 - protein, 11 aa, Artificial
  CDR sequence of cMET VHH
ALGNRWGTFPN

SEQ ID NO: 7 - protein, 13 aa, Artificial
  CDR sequence of cMET VHH
AFSGNFFNSYAMG

SEQ ID NO: 8 - protein, 17 aa, Artificial
  CDR sequence of cMET VHH
AISRSGLSTYYGDSVKG

SEQ ID NO: 9 - protein, 18 aa, Artificial
  CDR sequence of cMET VHH
AADRRSGSYFLRSTEWDY

SEQ ID NO: 10 - protein, 10 aa, Artificial
  CDR sequence of cMET VHH
GFTFDAFSIG

SEQ ID NO: 11 - protein, 17 aa, Artificial
  CDR sequence of cMET VHH
CINNNDGSIYYLDSVKG

SEQ ID NO: 12 - protein, 25 aa, Artificial
  CDR sequence of cMET VHH
AAVKRPGVRDHCYSIVAVRDYDMDY

SEQ ID NO: 13 - protein, 10 aa, Artificial
  CDR sequence of EGFR VH
GFTFSTYGMH

SEQ ID NO: 14 - protein, 17 aa, Artificial
  CDR sequence of EGFR VH
VIWDDGSYKYYGDSVKG

SEQ ID NO: 15 - protein, 18 aa, Artificial
  CDR sequence of EGFR VH
ARDGITMVRGVMKDYFDY

SEQ ID NO: 16 - protein, 11 aa, Artificial
  CDR sequence of EGFR VL
RASQDISSALV

SEQ ID NO: 17 - protein, 7 aa, Artificial
  CDR sequence of EGFR VL
DASSLES

SEQ ID NO: 18 - protein, 9 aa, Artificial
  CDR sequence of EGFR VL
QQFNSYPLT

SEQ ID NO: 19 - protein, 123 aa, Artificial
  cMET VHH sequence
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSS

SEQ ID NO: 20 - protein, 118 aa, Artificial
  cMET VHH sequence
QVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSS

SEQ ID NO: 21 - protein, 125 aa, Artificial
  cMET VHH sequence
QVQLVESVGGLVQTGGSLTLSCAFSGNFFNSYAMGWFRQAPGKEREFVAAISRSGLSTYYGDSVKGRFTISRDNAKNTVYLQMNSLKPEDTAVYYCAADRRSGSYFLRSTEWDYWGQGTLVTVSS

SEQ ID NO: 22 - protein, 132 aa, Artificial
  cMET VHH sequence
QVQLVESGGGLVQAGGSVRLSCAASGFTFDAFSIGWFRQAPGKEREGVSCINNNDGSIYYLDSVKGRFTISRDNAKNTAYLQMNSLEPEDTAVYYCAAVKRPGVRDHCYSIVAVRDYDMDYWGKGTLVTVSS

SEQ ID NO: 23 - protein, 125 aa, Artificial [sequence omitted]

SEQ ID NO: 24 - protein, 107 aa, Artificial [sequence omitted]

SEQ ID NO: 25 - protein, 15 aa, Homo sapiens
EPKSCDKTHTCPPCP

SEQ ID NO: 26 - protein, 15 aa, Artificial
  Fc hinge sequence
GGGGSDKTHTCPPCP

SEQ ID NO: 27 - protein, 15 aa, Artificial
  Fc hinge sequence
EPKSSDKTHTCPPCP

SEQ ID NO: 28 - protein, 10 aa, Artificial
  Fc hinge sequence
DKTHTCPPCP

SEQ ID NO: 29 - protein, 16 aa, Artificial
  Fc hinge sequence
STMVRSDKTHTCPPCP

SEQ ID NO: 30 - protein, 217 aa, Artificial
  Fc region sequence with knob mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 217 aa, Artificial
  Fc region sequence hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 32 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 34 - protein, 107 aa, Homo sapiens [sequence omitted]

SEQ ID NO: 35 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 36 - protein, 110 aa, Artificial
  Mutated human IgG1 CH2 domain
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 37 - protein, 5 aa, Artificial
  Linker peptide
GGGGS

SEQ ID NO: 38 - protein, 10 aa, Artificial
  Linker peptide
GGGGSGGGGS

SEQ ID NO: 39 - protein, 15 aa, Artificial
  Linker peptide
GGGGSGGGGSGGGGS

SEQ ID NO: 40 - protein, 20 aa, Artificial
  Linker peptide
GGGGSGGGGSGGGGSGGGGS

SEQ ID NO: 41 - protein, 25 aa, Artificial
  Linker peptide
GGGGSGGGGSGGGGSGGGGSGGGGS

SEQ ID NO: 42 - protein, 30 aa, Artificial
  Linker peptide
GGGGSGGGGSGGGGSGGGGSGGGGSGGGGS

SEQ ID NO: 43 - protein, 355 aa, Artificial
  Exemplary chain 1 sequence of cMET/EGFR BsAb in BiSpecific-V1 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 44 - protein, 355 aa, Artificial
  Exemplary chain 1 sequence of cMET/EGFR BsAb in BiSpecific-V1 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 45 - protein, 455 aa, Artificial
  Exemplary chain 2 sequence of cMET/EGFR BsAb in BiSpecific-V1 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 455 aa, Artificial
  Exemplary chain 2 sequence of cMET/EGFR BsAb in BiSpecific-V1 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, Artificial
  Exemplary chain 3 sequence of cMET/EGFR BsAb in BiSpecific-V1 format
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 488 aa, Artificial
  Exemplary chain 1 sequence of cMET/EGFR TsAb in TriSpecific-V1 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 49 - protein, 483 aa, Artificial
  Exemplary chain 1 sequence of cMET/EGFR TsAb in TriSpecific-V1 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 50 - protein, 455 aa, Artificial; identical to SEQ ID NO: 45
  Exemplary chain 2 sequence of cMET/EGFR TsAb in TriSpecific-V1 format

SEQ ID NO: 51 - protein, 455 aa, Artificial; identical to SEQ ID NO: 46
  Exemplary chain 2 sequence of cMET/EGFR TsAb in TriSpecific-V1 format

SEQ ID NO: 52 - protein, 214 aa, Artificial; identical to SEQ ID NO: 47
  Exemplary chain 3 sequence of cMET/EGFR TsAb in TriSpecific-V1 format

SEQ ID NO: 53 - protein, 355 aa, Artificial
  Exemplary chain 1 sequence of cMET/EGFR BsAb in BiSpecific-V2 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 355 aa, Artificial
  Exemplary chain 1 sequence of cMET/EGFR BsAb in BiSpecific-V2 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 479 aa, Artificial
  Exemplary chain 2 sequence of cMET/EGFR BsAb in BiSpecific-V2 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 56 - protein, 474 aa, Artificial
  Exemplary chain 2 sequence of cMET/EGFR BsAb in BiSpecific-V2 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 57 - protein, 488 aa, Artificial
  Exemplary chain 1 sequence of cMET/EGFR TsAb in TriSpecific-V2 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 483 aa, Artificial
  Exemplary chain 1 sequence of cMET/EGFR TsAb in TriSpecific-V2 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 479 aa, Artificial; identical to SEQ ID NO: 55
  Exemplary chain 2 sequence of cMET/EGFR TsAb in TriSpecific-V2 format

SEQ ID NO: 60 - protein, 474 aa, Artificial; identical to SEQ ID NO: 56
  Exemplary chain 2 sequence of cMET/EGFR TsAb in TriSpecific-V2 format

SEQ ID NO: 61 - protein, 593 aa, Artificial
  Exemplary chain 1 sequence of cMET/EGFR BsAb in BiSpecific-V3 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 588 aa, Artificial
  Exemplary chain 1 sequence of cMET/EGFR BsAb in BiSpecific-V3 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 214 aa, Artificial; identical to SEQ ID NO: 47
  Exemplary chain 2 sequence of cMET/EGFR BsAb in BiSpecific-V3 format

SEQ ID NO: 64 - protein, 593 aa, Artificial
  Exemplary chain 1 sequence of cMET/EGFR TsAb in TriSpecific-V3 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 588 aa, Artificial
  Exemplary chain 1 sequence of cMET/EGFR TsAb in TriSpecific-V3 format
QVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 588 aa, Artificial
  Exemplary chain 2 sequence of cMET/EGFR TsAb in TriSpecific-V3 format
QVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 67 - protein, 583 aa, Artificial
  Exemplary chain 2 sequence of cMET/EGFR TsAb in TriSpecific-V3 format
QVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 68 - protein, 214 aa, Artificial; identical to SEQ ID NO: 47
  Exemplary chain 3 sequence of cMET/EGFR TsAb in TriSpecific-V3 format

SEQ ID NO: 69 - protein, 593 aa, Artificial
  Exemplary chain 1 sequence of cMET/EGFR BsAb in BiSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSS

SEQ ID NO: 70 - protein, 588 aa, Artificial
  Exemplary chain 1 sequence of cMET/EGFR BsAb in BiSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSS

SEQ ID NO: 71 - protein, 214 aa, Artificial; identical to SEQ ID NO: 47
  Exemplary chain 2 sequence of cMET/EGFR BsAb in BiSpecific-V4 format

SEQ ID NO: 72 - protein, 593 aa, Artificial
  Exemplary chain 1 sequence of cMET/EGFR TsAb in TriSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSS

SEQ ID NO: 73 - protein, 588 aa, Artificial
  Exemplary chain 1 sequence of cMET/EGFR TsAb in TriSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQPGGSLKLSCAVSASLFSINTMGWFRQGPGKEREFVAAISWSGGNYYATFARGRFTISSEDAKDTVYLQMDNLKPEDTAVYFCGADRWGTGSMNAPDYDYWGRGTLVTVSS

SEQ ID NO: 74 - protein, 588 aa, Artificial
  Exemplary chain 2 sequence of cMET/EGFR TsAb in TriSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSS

SEQ ID NO: 75 - protein, 583 aa, Artificial
  Exemplary chain 2 sequence of cMET/EGFR TsAb in TriSpecific-V4 format
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSQVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSS

SEQ ID NO: 76 - protein, 214 aa, Artificial; identical to SEQ ID NO: 47
  Exemplary chain 3 sequence of cMET/EGFR TsAb in TriSpecific-V4 format

SEQ ID NO: 77 - protein, 123 aa, Artificial
  Humanized cMET VHH sequence
QVQLVESGGGLVQPGGSLRLSCAASASLFSINTMGWFRQAPGKGREFVAAISWSGGNYYATFARGRFTISRDNSKNTVYLQMNSLRAEDTAVYYCGADRWGTGSMNAPDYDYWGQGTLVTVSS

SEQ ID NO: 78 - protein, 123 aa, Artificial
  Humanized cMET VHH sequence
QVQLVESGGGLVQPGGSLRLSCAASASLFSINTMGWFRQAPGKEREFVAAISWSGGNYYATFARGRFTISRDNSKNTVYLQMESLRAEDTAVYYCGADRWGTGSMNAPDYDYWGQGTLVTVSS

SEQ ID NO: 79 - protein, 123 aa, Artificial
  Humanized cMET VHH sequence
EVQLVESGGGLVQPGGSLRLSCAASASLFSINTMGWFRQAPGKEREFVAAISWSGGNYYATFARGRFTISSDDSKDTVYLQMESLRAEDTAVYYCGADRWGTGSMNAPDYDYWGQGTLVTVSS

SEQ ID NO: 80 - protein, 123 aa, Artificial
  Humanized cMET VHH sequence
QVQLVESGGGLVQPGGSLRLSCAVSASLFSINTMGWFRQGPGKEREFVAAISWSGGNYYATFARGRFTISRDNSKNTVYLQMESLRAEDTAVYFCGADRWGTGSMNAPDYDYWGQGTLVTVSS

SEQ ID NO: 81 - protein, 123 aa, Artificial
  Humanized cMET VHH sequence
QVQLVESGGGLVQPGGSLRLSCAVSASLFSINTMGWFRQGPGKEREFVAAISWSGGNYYATFARGRFTISSDNSKNTVYLQMESLRAEDTAVYFCGADRWGTGSMNAPDYDYWGQGTLVTVSS

SEQ ID NO: 82 - protein, 132 aa, Artificial
  Humanized cMET VHH sequence
QVQLVESGGGLVQPGGSLRLSCAASGFTFDAFSIGWFRQAPGKEREGVSCINNNDGSIYYLDSVKGRFTISRDNSKNTAYLQMNSLRAEDTAVYYCAAVKRPGVRDHCYSIVAVRDYDMDYWGQGTLVTVSS

SEQ ID NO: 83 - protein, 132 aa, Artificial
  Humanized cMET VHH sequence
QVQLVESGGGLVQPGGSVRLSCAASGFTFDAFSIGWFRQAPGKEREGVSCINNNDGSIYYLDSVKGRFTISRDNSKNTAYLQMNSLRAEDTAVYYCAAVKRPGVRDHCYSIVAVRDYDMDYWGQGTLVTVSS

SEQ ID NO: 84 - protein, 123 aa, Artificial
  Humanized cMET VHH sequence
QVQLVESGGGLVQPGGSLRLSCAASASLFSINTMGWFRQAPGKEREFVAAISWSGGNYYATFARGRFTISSEDSKDTVYLQMESLRAEDTAVYYCGADRWGTGSMNAPDYDYWGQGTLVTVSS

SEQ ID NO: 85 - protein, 588 aa, Artificial
  Exemplary chain 1 sequence of cMET/EGFR TsAb in TriSpecific-V3 format
QVQLVESGGGLVQPGGSLRLSCAASGFTFNSYWMYWVRLAPGKGIEWVSATDTRGQNTYYTDSVKGRFTISRDNAKNMVSLQMNNLKSEDTAVYYCALGNRWGTFPNRGPGTLVTVSSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 593 aa, Artificial
  Exemplary chain 2 sequence of cMET/EGFR TsAb in TriSpecific-V3 format
QVQLVESGGGLVQPGGSLRLSCAASASLFSINTMGWFRQAPGKEREFVAAISWSGGNYYATFARGRFTISSEDSKDTVYLQMESLRAEDTAVYYCGADRWGTGSMNAPDYDYWGQGTLVTVSSGGGGSGGGGSGGGGSQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK